(19) United States
(12) Patent Application Publication
(10) Pub. No.: US 2017/0307619 A1
(43) Pub. Date: Oct. 26, 2017

(54) MARKERS OF POOR PROGNOSIS ACUTE MYELOID LEUKEMIAS (AMLs) AND USES THEREOF

(71) Applicants: UNIVERSITÉ DE MONTRÉAL, MONTREAL (CA); RSEM, LIMITED PARTNERSHIP, MONTREAL (CA)

(72) Inventors: GUY SAUVAGEAU, MONTREAL (CA); VINCENT-PHILIPPE LAVALLÉE, MONTREAL (CA); JOSÉE HÉBERT, MONT-ROYAL (CA); SÉBASTIEN LEMIEUX, LASALLE (CA); BERNHARD LEHNERTZ, MONTREAL (CA)

(21) Appl. No.: 15/516,081

(22) PCT Filed: May 21, 2015
(86) PCT No.: PCT/CA2015/050462

§ 371 Date: Mar. 31, 2017

Related U.S. Application Data
(60) Provisional application No. 62/058,916, filed on Oct. 2, 2014.

(51) Int. Cl. G01N 33/574; C07H 21/04; C40B 30/04; C12Q 1/68; G01N 33/50

(52) U.S. Cl. CPC G01N 33/57426; C12Q 1/6886; G01N 33/50; C40B 30/04; C07H 21/04; C12Q 2600/118; G01N 2800/52; C12Q 2600/158

(57) ABSTRACT

Genes exhibiting specific mutational and/or transcriptional patterns in poor prognosis AMLs, such as EVI1-rearranged acute myeloid leukemias (EVI1-r AMLs), relative to other types of AMLs and/or normal CD34+ cells, are disclosed. The use of these mutational and/or transcriptional patterns, for example the expression level of the PRKC Apoptosis WT1 Regulator (PAWR) gene, for the diagnosis or prognosis of AMLs, including intermediate-risk AMLs, is also disclosed.

### CROSS REFERENCE TO RELATED APPLICATIONS

The present application claims the benefits of U.S. provisional application Ser. No. 62/058,916 filed on Oct. 2, 2014, which is incorporated herein by reference in its entirety.

### FIELD OF THE INVENTION

The present invention relates to acute myeloid leukemias (AMLs), and more particularly to the diagnosis/prognosis of poor prognosis AMLs such as EVI1-rearranged acute myeloid leukemias (EVI1-r AMLs).

### BACKGROUND OF THE INVENTION

Acute Myeloid Leukemia (AML) is a particularly lethal form of cancer, with most patients dying within two years of diagnosis. It is one of the leading causes of death among young adults. AML is a collection of neoplasms with heterogeneous pathophysiology, genetics and prognosis. Mainly based on cytogenetics and molecular analysis, AML patients are presently classified into groups or subsets of AML with markedly contrasting prognosis. Approximately 45% of all AML patients are currently classified into distinct groups with variable prognosis based on the presence or absence of specific recurrent cytogenetic abnormalities. A significant proportion of patients, however, are classified as intermediate-risk AML patients, accounting for approximately 55% of all AML patients. Existing AML prognostic tests are often inaccurate, leaving hemato-oncologists with a lack of tools to guide their decision making about treatment options (consolidation chemotherapy, allogeneic hematopoietic stem cell transplantation and/or more aggressive treatment).

The EVI1 (Ecotropic Viral Integration Site 1) gene, also termed MECOM (MDS1 and EVI1 complex locus), is located on chromosomal band 3q26.2. It is expressed at high levels in normal CD34⁺ cells¹ and in 5-10% of human AML²⁻⁴. In a subset of these patients, EVI1 is rearranged. The typical cytogenetic anomaly inv(3)(q21q26.2)/t(3;3)(q21;q26.2), included in 2008 in the WHO classification under the category “AML with recurrent genetic abnormalities”, represents one such rearrangement. This entity is characterized by a very poor overall survival and by distinct morphologic features including atypical megakaryocytes, multilineage dysplasia, and normal or elevated blood platelet counts⁵⁻⁷.

The most frequent cytogenetic anomaly associated to EVI1-rearranged (hereinafter EVI1-r) AMLs is monosomy 7, found in 33-66% of cases of inv(3)/t(3;3)⁶⁻¹⁰ and in one third of AML with other EVI1 rearrangements⁷. Targeted analyses have revealed mutations in RUNX1, NRAS, KRAS, and NF1 in 20 to 33% of cases⁹. However, the full mutation spectrum remains unknown for this rare disease.

EVI1 is an oncogene with numerous splice variants of which the MDS1/EVI1 is the longest isoform¹¹. It encodes for a protein in which the N-terminal region includes an additional 188 residues derived from MDS1 genes and forming a PR (proline rich) domain. This isoform is believed to antagonize the transforming function of the shorter isoforms lacking this PR domain (sometimes collectively referred to as cEVI1: reviewed in ¹¹). The relative expression levels of the short isoforms of EVI1 have been reported in AML^{3,12} but not in normal human cord blood (CB)-derived CD34⁺ cells.

Thus, there is a need for a better characterization of the genetic and transcriptional signature of AMLs such as EVI1-r AMLs, and for the identification of markers useful for the diagnosis and/or prognosis of AML, including intermediate-risk AML, i.e. to better predict treatment outcome and/or survival.

The present description refers to a number of documents, the content of which is herein incorporated by reference in their entirety.

### SUMMARY OF THE INVENTION

More specifically, in accordance with the present invention, there is provided the following items 1 to 53:

1. A method for the disease prognosis of a subject suffering from acute myeloid leukemia (AML), said method comprising: measuring the level of expression of PAWR in a biological sample comprising leukemic cells from said subject; and comparing said level of expression to a threshold reference level, wherein a level of expression that is above said threshold reference level is indicative of a poor disease prognosis.

2. The method of item 1, wherein said the level of expression is measured at the nucleic acid level.

3. The method of item 2, wherein said method comprises amplifying a nucleic acid encoding PAWR using a first PAWR primer and a second PAWR primer.

4. The method of item 3, wherein said first PAWR primer comprises at least 10 nucleotides of the sequence 5′-TGGTCAACATCCCTGCCG-3′ (SEQ ID NO: 3).

5. The method of item 4, wherein said first PAWR primer comprises the sequence 5′-TGGTCAACATCCCTGCCG-3′ (SEQ ID NO: 3).

6. The method of any one of items 3 to 5, wherein said second PAWR primer comprises at least 10 nucleotides of the sequence 5′-TTGCATCTTCTCGTTTCCGC-3′ (SEQ ID NO: 4).

7. The method of item 6, wherein said second PAWR primer comprises the sequence 5′-TTGCATCTTCTCGTTTCCGC-3′ (SEQ ID NO: 4).

8. The method of any one of items 2 to 7, wherein said method comprises detecting the nucleic acid encoding PAWR using a PAWR probe.

9. The method of item 8, wherein said PAWR probe comprises at least about 10 nucleotides of the sequence 5′-AGTACGAAGATGATGAAGCAGGGC-3′ (SEQ ID NO: 6).

10. The method of item 9, wherein said PAWR probe comprises the sequence 5′-AGTACGAAGATGATGAAGCAGGGC-3′ (SEQ ID NO: 6).

11. The method of any one of items 2 to 10, wherein the level of expression of PAWR is measured by reverse transcription polymerase chain reaction (RT-PCR), for example quantitative reverse transcription polymerase chain reaction (RT-qPCR).

12. The method of any one of items 1 to 11, wherein said method further comprises normalizing the level of expression of PAWR based on the level of expression of a housekeeping gene.

13. The method of item 12, wherein said housekeeping gene is ABL1.

14. The method of item 13, wherein said method comprises amplifying a nucleic acid encoding ABL1 using a first ABL1 primer and a second ABL1 primer.

15. The method of item 14, wherein said first ABL1 primer comprises at least 10 nucleotides of the sequence 5′-TGGAGATAACACTCTAAGCATAACTAAAGGT-3′ (SEQ ID NO: 1).

16. The method of item 15, wherein said first ABL1 primer comprises the sequence 5′-TGGAGATAACACTCTAAGCATAACTAAAGGT-3′ (SEQ ID NO: 1).

17. The method of any one of items 14 to 16, wherein said second ABL1 primer comprises at least 10 nucleotides of the sequence 5′-GATGTAGTTGCTTGGGACCCA-3′ (SEQ ID NO: 2).

18. The method of item 17, wherein said second ABL1 primer comprises the sequence 5′-GATGTAGTTGCTTGGGACCCA-3′ (SEQ ID NO: 2).

19. The method of any one of items 12 to 18, wherein said method comprises detecting the nucleic acid encoding ABL1 using an ABL1 probe.

20. The method of item 19, wherein said ABL1 probe comprises at least about 10 nucleotides of the sequence 5′-CCATTTTTGGTTTGGGCTTCACACCATT-3′ (SEQ ID NO: 5).

21. The method of item 20, wherein said ABL1 probe comprises the sequence 5′-CCATTTTTGGTTTGGGCTTCACACCATT-3′ (SEQ ID NO: 5).

22. The method of any one of items 1 to 21, further comprising measuring the level of expression of at least one additional prognostic marker gene in said biological sample.

23. The method of any one of items 1 to 22, wherein said biological sample is a peripheral blood sample or a bone marrow sample.

24. The method of any one of items 1 to 23, wherein said poor disease prognosis comprises low probability of survival.

25. The method of any one of items 1 to 24, wherein said AML is an intermediate-risk AML.

26. The method of item 25, wherein said intermediate-risk is FLT3-ITD negative AML.

27. A method for determining the likelihood that a subject suffers from EVI1-rearranged acute myeloid leukemia (EVI1-r AML), said method comprising: determining the presence of one or more of the mutations depicted in FIG. 5A in a leukemia cell sample from said subject: wherein the presence of said one or more mutations is indicative that said subject has a high likelihood of suffering from EVI1-r AML, and wherein the absence of said one or more mutations is indicative that said subject has a low likelihood of suffering from EVI1-r AML.

28. The method of item 27, wherein said one or more mutations is in a member of the RAS pathway.

29. The method of item 28, wherein said one or more mutations is: a G to C or G to D substitution at a position corresponding to amino acid 12 of NRAS; a G to D substitution at a position corresponding to amino acid 13 of NRAS; a Q to K substitution at a position corresponding to amino acid 61 of NRAS; a G to D substitution at a position corresponding to amino acid 12 KRAS; a D to V substitution at a position corresponding to amino acid 61 of PTPN11; an E to K substitution at a position corresponding to amino acid 69 of PTPN11; an A to V substitution at a position corresponding to amino acids 72 of PTPN11; a V to D substitution at a position corresponding to amino acids 1419 of NF1 and/or; a mutation causing a frameshift at a position corresponding to amino acids 2423 of NF1.

30. The method of item 27, wherein said one or more mutations is in IKZF1.

31. The method of item 30, wherein said one or more mutations is: an N to S substitution at a position corresponding to amino acid 159 of IKZF1; an R to STOP substitution at a position corresponding to amino acid 213 of IKZF1; and/or a mutation causing a frameshift at a position corresponding to amino acid 270 of IKZF1.

32. The method of item 27, wherein said one or more mutations is in a splicing factor.

33. The method of item 32, wherein said one or more mutations is: an R to C substitution at a position corresponding to amino acid 625 of SF3B1; a K to E substitution at a position corresponding to amino acid 700 of SF3B1; a G to E substitution at a position corresponding to amino acid 740 of SF3B1; a Q to R substitution at a position corresponding to amino acid 157 of U2AF1; and/or a Q to P substitution at a position corresponding to amino acid 157 of U2AF1.

34. The method of item 27, wherein said one or more mutations is in TP53.

35. The method of item 34, wherein said one or more mutations is: an Y to C substitution at a position corresponding to amino acid 205 of TP53; and/or an R to W substitution at a position corresponding to amino acid 248 of TP53.

36. The method of item 27, wherein said one or more mutations is in ASXL1.

37. The method of item 36, wherein said one or more mutations is a mutation causing a frameshift at a position corresponding to amino acid 643 of ASXL1.

38. The method of item 27, wherein said one or more mutations is in DNMT3A.

39. The method of item 38, wherein said one or more mutations is an R to C substitution at a position corresponding to amino acid 882 of DNMT3A.

40. The method of item 27, wherein said one or more mutations is in ETV6.

41. The method of item 40, wherein said one or more mutations is an I to F substitution at a position corresponding to amino acid 406 of ETV6.

42. The method of item 27, wherein said one or more mutations is in KIT.

43. The method of item 42, wherein said one or more mutations is a D to V substitution at a position corresponding to amino acid 816 of KIT.

44. The method of any one of items 27 to 43, wherein the presence of said one or more mutations is determined by sequencing a region encompassing said one or more mutations in a nucleic acid present in said sample.

45. The method of item 44, wherein said nucleic acid is cDNA.

46. The method of item 45, wherein said sequencing is performed by RNA sequencing (RNAseq).

47. A method for determining the likelihood that a subject suffers from EVI1-rearranged acute myeloid leukemia (EVI1-r AML), said method comprising: determining the level of expression of at least one of the genes depicted in Table 2 in a leukemia cell sample from said subject: wherein a higher expression of said at least one genes in said sample relative to a control non-EVI1-r AML sample, is indicative that said subject has a high likelihood of suffering from EVI1-r AML.

48. The method of item 47, wherein said method comprises determining the level of expression of at least one MECOM isoform.

49. The method of item 48, wherein said at least one MECOM isoform is MECOM isoform 16 (SEQ ID NO: 10) and/or MECOM isoform 5 (SEQ ID NO: 9).

50. The method of item 47, wherein said method comprises determining the level of expression of at least one of MECOM isoform 1d, MECOM isoform 1a, VIP, PREX2, MYCT1 and PAWR.

51. A method for determining the likelihood that a subject suffers from EVI1-rearranged acute myeloid leukemia (EVI1-r AML), said method comprising: determining the level of expression of at least one of the genes depicted in FIG. 6B and FIG. 6C in a leukemia cell sample from said subject: wherein a higher expression of said at least one genes depicted in FIG. 6B, and/or a lower expression of said at least one genes depicted in FIG. 6C, in said sample relative to a control CD34+ cell sample, is indicative that said subject has a high likelihood of suffering from EVI1-r AML.

52. The method of any one of items 47 to 51, wherein said the level of expression is measured at the nucleic acid level.

53. The method of item 52, wherein said the level of expression is measured by RNA sequencing (RNAseq) or reverse transcription polymerase chain reaction (RT-PCR), for example quantitative reverse transcription polymerase chain reaction (RT-qPCR).

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1A shows the clinical, morphologic, cytogenetic and mutational data in EVI1-r AMLs samples.

FIG. 1B shows the commonly mutated pathways in EVI1-r AMLs samples.

FIG. 1C shows the IKZF1 protein and mutations. Four N-terminal (DNA-binding domain) and two C-terminal (dimerization domain) zinc fingers are represented by cylinders.

FIG. 1D shows the distribution of IKZF1 isoforms in EVI1-r samples. To avoid issues with log-scale representation of FPKM equal to zero, a small constant (0.0001) was added to all expression values. The only sample expressing isoform NM_001220772 (Ik6) harbors compound heterozygous mutations of IKZF1.

FIG. 1E shows IKZF1 expression levels in EVI1-r AMLs with and without monosomy 7.

FIG. 1F shows the morphology, immunochemistry, and flow cytometry of IKZF1 mutated samples.

FIG. 2A shows a comparative analysis of expressed genes in EVI-r AMLs compared to AML without EVI1 rearrangements. For normalization, a value of 0.0001 was added to the RPKM values prior to log 10 transformation. Among expressed genes (i.e. with an average log 10 expression ≧3, corresponding approximately to an expression of 0.1 RPKM), 20 genes with the greatest differential expression in EVI1-r AML are highlighted (solid rhombuses, also detailed in FIG. 6A and Table 2).

FIG. 2B shows a comparative analysis of expression of MECOM, PREX2, MYCT1, VIP and PAWR between EVI1-r AML, AML without EVI1 rearrangements and normal CD34+ cells. p-value: *<0.05, **<0.005 and ***<0.0005.

FIG. 2C shows a schematic representation of MDS1 and EVI1 5′ end (upper panel), and the expression of MECOM splice isoforms in EVI1-r AMLs, other AMLs expressing MECOM (n=16) and normal CD34⁺ cells (lower panel). p-value: *<0.05, **<0.005 and ***<0.0005.

FIGS. 2D to 2M show cufflinks isoform expression of MECOM_iso_16 (FIG. 2D), MECOM_iso_22 (FIG. 2E), MECOM_iso_20 (FIG. 2F), MECOM_iso_29 (FIG. 2G), MECOM_iso_28 (FIG. 2H), MECOM_iso_5 (FIG. 2I), MECOM_iso_21 (FIG. 2J), MYCT1_iso_6 (FIG. 2K), LRBA_iso_6 (FIG. 2L) and LRBA_iso_3 (FIG. 2M), in AML subtypes and normal hematopoietic populations. Ab initio transcriptome assembly based on raw sequence data using Tophat/Cufflinks identifies the above-noted isoforms. Summary dotplot of various AML genetic subtypes and normal hematopoietic populations from bone marrow, cord blood, and peripheral blood show the indicated transcripts as robustly expressed in the majority of EVI1-rearranged AMLs (MECOM_iso_16 (FIG. 2D), MECOM_iso_22 (FIG. 2E), MECOM_iso_20 (FIG. 2F), MECOM_iso_29 (FIG. 2G), MECOM_iso_28 (FIG. 2H), MECOM_iso_5 (FIG. 2I), MECOM_iso_21 (FIG. 2J), MYCT1_iso_6 (FIG. 2K), LRBA_iso_6 (FIG. 2L) and LRBA_iso_3 (FIG. 2M). Each dot represents FPKM expression for one specimen and median values are indicated by a horizontal line. A straight line demarcates an FPKM of 1.0. A value of 0.0001 was added to all FPKM values in order to apply log₁₀ transformation.

FIGS. 3A to 3G show the characteristics of the cohort of AMLs from other cytogenetic groups, including sequencing information.

FIG. 3H shows the sequencing and mapping statistics of 12 EVI1-rearranged samples.

FIGS. 4A to 4C show a list of cancer-associated genes. Cancer 5000: Lawrence M, Stojanov P, Mermel C, et al. Discovery and saturation analysis of cancer genes across 21 tumour types. Nature. 2014; 505(7484):495-501; Other: genes associated or possibly associated to hematologic cancers in other datasets.

FIGS. 5A to 5C show the variants identified in the studies described herein. FIG. 5A: All mutations (proven not germline or in known positions) as seen in FIG. 1A. FIG. 5B: Germline or unknown variants in cancer genes. FIG. 5C: Germline or unknown variants in non-cancer genes.

FIG. 6A shows the data for the 20 genes exhibiting the highest difference of expression between EVI1-r AML and non EVI1-r AML samples.

FIG. 6B shows the genes exhibiting the highest expression in EVI1-r AML relative to normal CD34+ cell samples.

FIG. 6C shows the genes exhibiting the lowest expression in EVI1-r AML relative to normal CD34+ cell samples.

FIG. 7 shows the correlation between transcriptome (RNA-seq) data and the real-time quantitative RT-PCR assay for PAWR. The variation between RNA-Seq transcriptome data and the developed PAWR quantitative RT-PCR assay was compared. The resultant scatter plot shows robust correlation in PAWR expression levels detected using either RNA-Seq transcriptome data (log RPKM) or the developed quantitative RT-PCR assay (log₁₀ values of (normalized PAWR copy number per 10⁴ ABL1 copy number, NCN)). To avoid issues with log-scale representation of RPKM equal to zero, a small constant (0.0001) was added to all RNA-Seq expression values.

FIGS. 8A and 8B show that PAWR quantitative RT-PCR shows a large dynamic range between genetic subtypes in the Leucegene AML cohort. FIG. 8A: AML specimens assessed by the PAWR quantitative RT-PCR assay were separated according to normalized values of PAWR copy number per 10⁴ ABL1 copy number and subjected to Kaplan-Meier survival probability analysis. A significant difference in overall survival was observed in samples expressing greater than or equal to 1000 normalized copy numbers, thereby identifying a potential cut-off value. FIG. 8B: Dot plot analysis shows the expression levels of genetic subtypes within the Leucegene AML cohort as determined using the PAWR quantitative RT-PCR assay. Each triangle represents PAWR expression for one specimen, reported as normalized copy number. The previously identified cut-off at 1000 normalized copy number is indicated by the dotted line. Greater than 1000 normalized copy number expression is associated with genetic subtypes with known adverse clinical outcome, however, several additional specimens are identified with expression above cut-off but are either normal karyotype, intermediate abnormal AML, or otherwise not associated with genetic subgroups of poor clinical outcome. Complex karyotype without TP53 mutation or deletion (TP53 wt), n=20; Complex karyotype with TP53 mutation and/or deletion, n=22; Normal karyotype (NK), n=117; Intermediate abnormal karyotype (Interm.abn.), n=50; Trisomy 8 alone, n=12; MLL fusions, n=27; t(8:21), n=17; inv(16), n=28; EVI1r, n=6; Monosomy 5/5q—or monosomy 7/7q—not complex (NC), n=7; NUP98-NSD1 in AML with normal karyotype (NK), n=5; t(6;9), n=2. Median values are indicated by a horizontal line. Abbreviation: EVIr, AML with EVI1 rearrangements.

FIGS. 9A and 9B show the overall survival curves according to PAWR expression in Leucegene de novo AML Intermediate Risk FLT3-ITD-negative cohort. Kaplan-Meier survival probability analysis on Intermediate Risk FLT3-ITD-negative (ITD−) AML based upon PAWR expression determined by RNA-Seq (FIG. 9A) and a quantitative RT-PCR assay (FIG. 9B) shows poor overall survival by specimens whose PAWR expression is >1 RPKM (FIG. 9A) or ≧1000 normalized (log₁₀) values of PAWR copy number per 10⁴ ABL1 copy number (FIG. 9B). Leucegene de novo AML Intermediate Risk cohort includes, intermediate abnormal karyotype, some MLL fusions (e.g. t(9;11)/MLL-MLLT3), Normal Karyotype, NUP98-NSD1 fusion in AML with normal karyotype, and trisomy 8 alone. FLT3-ITD and NUP98-NSD1 status were determined by Next Generation Sequencing (NGS).

FIGS. 9C and 9D show the overall survival curves based upon PAWR expression in Leucegene de novo AML cohort. Kaplan-Meier survival probability analysis based upon PAWR expression determined by the quantitative RT-PCR assay shows poor overall survival by specimens whose PAWR expression is ≧1000 normalized copy number (NCN) vs. specimens <1000 NCN. In FIG. 9C, AML patients with a favorable prognosis (t(8;21) and inv(16)), and for which the test is less informative (AML with MLL fusions), were excluded from the survival analysis.

FIGS. 10A and 10B shows the cDNA (SEQ ID NO: 7) and protein (SEQ ID NO: 8) sequences of human PAWR. The coding sequence is indicated in bold in FIG. 10A, and the nucleotides corresponding to the primers and probes used in the RT-PCR experiments described herein are underlined (the hybridized sequence is underlined in the case of the reverse primer).

### DETAILED DESCRIPTION OF THE INVENTION

Terms and symbols of genetics, molecular biology, biochemistry and nucleic acids used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like. All terms are to be understood with their typical meanings established in the relevant art.

The articles “a” and “an” are used herein to refer to one or to more than one (i.e. to at least one) of the grammatical object of the article. By way of example, “an element” means one element or more than one element. Throughout this specification, unless the context requires otherwise, the words “comprise,” “comprises” and “comprising” will be understood to imply the inclusion of a stated step or element or group of steps or elements but not the exclusion of any other step or element or group of steps or elements.

In the studies described herein, the present inventors have shown that EVI1-r AMLs exhibit distinct mutational and transcriptional signatures relative to normal CD34⁺ cells and other AML subtypes, which may be useful for the characterization, diagnosis and prognosis of EVI1-r AMLs. The present inventors have also shown that one of the markers overexpressed in EVI1-r AMLs, PAWR, constitutes a suitable prognostic marker for AML.

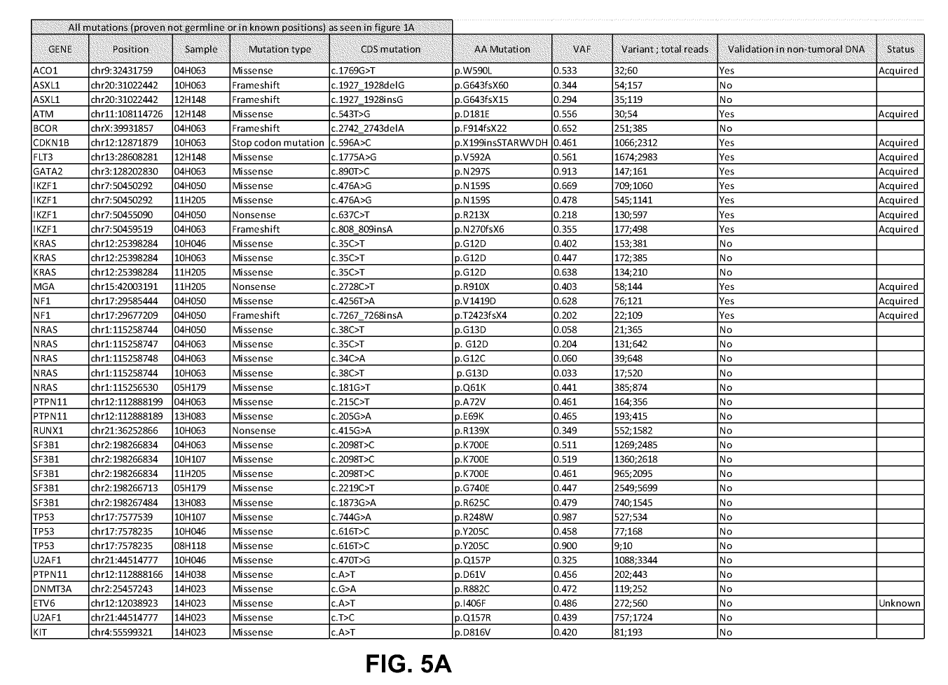

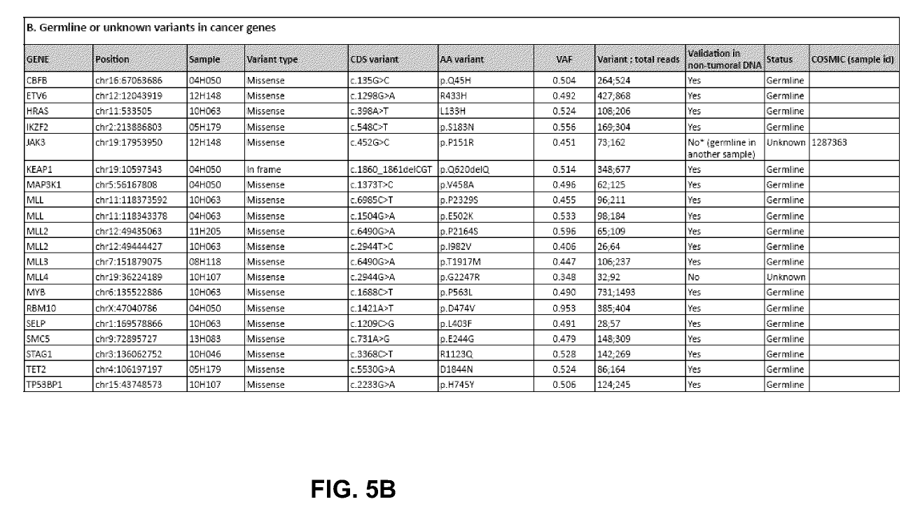

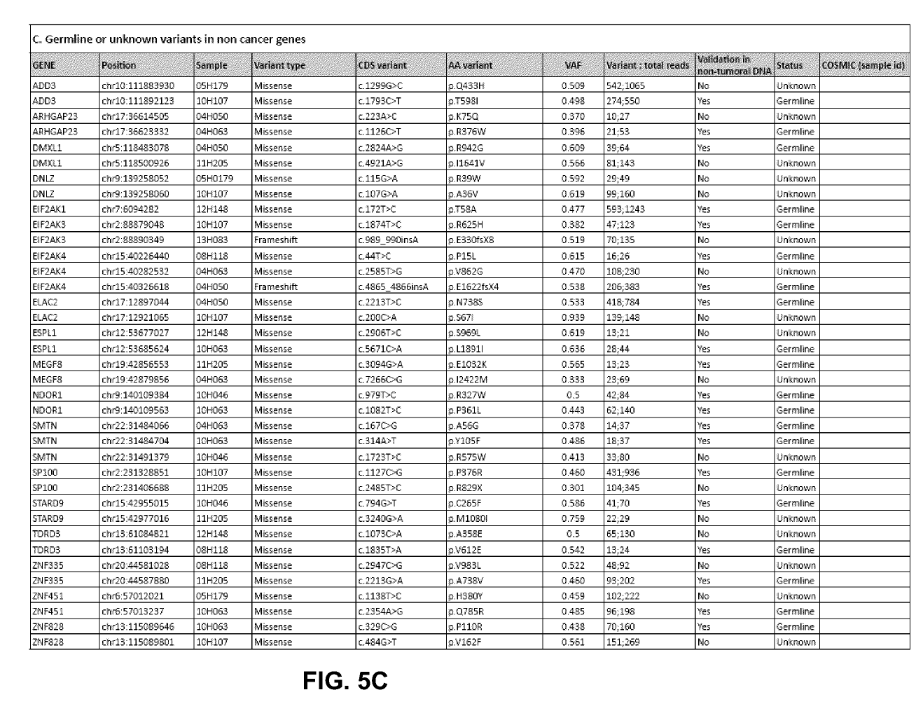

In an aspect, the present invention relates to a method for determining the likelihood that a subject suffers from EVI1-rearranged acute myeloid leukemia (EVI1-r AML), said method comprising: determining the presence of one or more of the mutations depicted in FIG. 5A in a leukemia cell (e.g., blood cell) sample from said subject: wherein the presence of said one or more mutations is indicative that said subject has a high likelihood of suffering from EVI1-r AML, and wherein the absence of said one or more mutations is indicative that said subject has a low likelihood of suffering from EVI1-r AML.

In another aspect, the present invention relates to a method for determining whether the likelihood that an AML sample is an EVI1-r AML sample, said method comprising: determining the presence of one or more of the mutations depicted in FIG. 5A said AML sample: wherein the presence of said one or more mutations is indicative that said sample has a high likelihood of being an EVI1-r AML sample, and wherein the absence of said one or more mutations is indicative that said sample has a low likelihood of being an EVI1-r AML sample (i.e. said sample is likely another type of AML sample).

The present invention encompasses the detection of any mutation or any combination/sub-combination of the mutations defined herein (FIG. 5A), for example the detection of a single mutation, or of 2, 3, 4, 5 or more of the mutations defined herein.

The Genbank, RefSeq or NCBI accession numbers corresponding to the genes mutated in FIG. 5A are indicated in Table 1 below. The information, including the nucleotide and amino acid sequences, corresponding to the Genbank, RefSeq or NCBI accession numbers referred to in the present specification is incorporated herein by reference.

TABLE 1

| Gene | Genbank or RefSeq accession | Gene | Genbank or RefSeq accession |
|---|---|---|---|
| name | Protein/cDNA/genomic | name | Protein/cDNA/genomic |
| ACO1 | NP_001265281.1/NM_001278352.1/ | MGA | NP_001157745.1/NM_001164273.1/ |
| | Gene ID: 48 | | Gene ID: 23269 |
| ASXL1 | NP_056153.2/NM_015338.5/ | NF1 | NP_001035957.1/NM_001042492.2/ |
| | NG_027868.1 | | NG_009018.1 |
| ATM | NP_000042.3/NM_000051.3/ | NRAS | NP_002515.1/NM_002524.4/ |
| | NG_009830.1 | | NG_007572.1 |
| BCOR | NP_060215.4/NM_017745.5/ | PTPN11 | NP_002825.3/NM_002834.3/ |
| | NG_008880.1 | | NG_007459.1 |
| CDKN1B | NP_004055.1/NM_004064.4/ | RUNX1 | NP_001745.2/NM_001754.4/ |
| | NG_016341.1 | | NG_011402.2 |
| FLT3 | NP_004110.2/NM_004119.2/ | SF3B1 | NP_036565.2/NM_012433.2/ |
| | NG_007066.1 | | NG_032903.2 |
| GATA2 | NP_116027.2/NM_032638.4/ | TP53 | NP_000537.3/NM_000546.5/ |
| | NG_029334.1 | | NG_017013.2 |
| IKZF1 | NP_006051.1/NM_006060.5/ | U2AF1 | NP_006749.1/NM_006758.2/ |
| | NG_034231.1 | | NG_029455.1 |
| KRAS | NP_203524.1/NM_033360.3/ | DNMT3A | NP_072046/NM_022552.4/Gene ID: |
| | NG_007524.1 | | 1788 |
| ETV6 | NP_001978.1/NM_001987.4/Gene | KIT | NP_000213.1/NM_000222.2/Gene |
| | ID: 2120 | | ID: 3815 |

As used herein, the term “high likelihood” means that the individual is more likely to have the disorder or disease (EVI1-r AML) than an individual without the mutation, or that the sample is more likely to be an EVI1-r AML sample than an AML sample without the mutation.

As used herein, the term “EVI1-r AML” refers to an acute myeloid leukemia EVI1 is rearranged. The typical cytogenetic anomaly inv(3)(q21q26.2)/t(3;3)(q21;q26.2), included in 2008 in the WHO classification under the category “AML with recurrent genetic abnormalities”, represents one example of such rearrangement. Other rearrangements in which EVI1 and/or MDS1/EVI1 involvement have been established include ins(3)(q26;q21q26), t(3;12)(q26;p13), t(3;21)(q26;q22), t(2;3)(p13-p23;q26), t(3;6)(q26;q25), t(3;13)(q26;q14), t(3;17)(q26;q22), inv(3)(p12q26), inv(3)(q23q26), t(3;3)(p24;q26), t(3;5)(q26;q34), t(3;9)(q26;p23), t(3;12)(q26;q21) and t(3;18)(q26;q11) (see, e.g., Poppe, B; Dastugue, N; Speleman, F, 3q rearrangements in myeloid malignancies, Atlas Genet Cytogenet Oncol Haematol. 2003; 7(2):110-112).

The determination of the presence of the mutation(s) in the sample may be performed using any suitable methods (see, e.g., Syvänen, Nat Rev Genet. 2001 December; 2(12):930-42). For example, the presence of the mutation(s) may be detected at the genomic DNA, transcript (RNA or cDNA) or protein level. Examples of suitable methods for determining sequences and polymorphisms at the nucleic acid level include sequencing of the nucleic acid sequence encompassing the mutation(s), e.g., in the genomic DNA or transcript (cDNA), for example by “Next Generation Sequencing” methods (e.g., genome sequencing, RNA sequencing (RNA-seq)) or other sequencing methods; hybridization of a nucleic acid probe capable of specifically hybridizing to a nucleic acid sequence comprising the mutation(s) and not to (or to a lesser extent to) a corresponding nucleic acid sequence that does not comprises the mutation(s) (under comparable hybridization conditions, such as stringent hybridization conditions) (e.g., molecular beacons); restriction fragment length polymorphism analysis (RFLP); Amplified fragment length polymorphism PCR (AFLP-PCR); amplification of a nucleic acid fragment comprising the mutation(s) using a primer specifically hybridizing to a nucleic acid sequence comprising the mutation(s), wherein the primer produces an amplified product if the mutation(s) is/are present and does not produce the same amplified product when a nucleic acid sequence not comprising the mutation(s) is used as a template for amplification, nucleic acid sequence based amplification (Nasba), primer extension assay, FLAP endonuclease assay (Invader assay, Olivier M. (2005). Mutat Res. 573(1-2):103-10), 5′ nuclease assay (McGuigan F. E. and Ralston S. H. (2002) Psychiatr Genet. 12(3):133-6), oligonucleotide ligase assay. Other methods include in situ hybridization analyses and single-stranded conformational polymorphism analyses. Several SNP genotyping platforms are commercially available. Additional methods will be apparent to one of skill in the art.

The determination of the presence of the mutation(s) may also be achieved at the polypeptide/protein level. Examples of suitable methods for detecting alterations at the polypeptide level (including polypeptides encoded by splice variants) include sequencing of the encoded polypeptide; digestion of the encoded polypeptide followed by mass spectrometry or HPLC analysis of the peptide fragments, wherein the mutated polypeptide results in an altered mass spectrometry or HPLC spectrum as compared to the unmutated polypeptide; and immunodetection using an immunological reagent (e.g., an antibody, a ligand) which exhibits altered immunoreactivity with a mutated polypeptide relative to a corresponding unmutated polypeptide. Immunodetection can measure the amount of binding between a polypeptide molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots, and other techniques known to those of ordinary skill in the art (see Harlow and Lane, Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). Methods to generate antibodies exhibiting altered immunoreactivity with a mutated polypeptide relative to a corresponding unmutated polypeptide are described in more detail below.

All these detection techniques may also be employed in the format of microarrays (e.g., SNP microarrays), protein-arrays, antibody microarrays, tissue microarrays, electronic biochip or protein-chip based technologies (see Schena M., Microarray Biochip Technology, Eaton Publishing, Natick, Mass., 2000).

Further, nucleic acid-containing sequences may be amplified prior to or in conjunction with the detection methods noted herein. The design of various primers for such amplification is known in the art. For example, a nucleic acid (RNA, cDNA, genomic DNA) comprising the mutation(s) may be amplified using primers hybridizing to sequences located on each side of the mutation(s). Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188. In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer that is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the mutation(s) to be detected is/are present. Detection of the amplified sequence may be carried out by visualization following Ethidium Bromide (EtBr) staining of the DNA following gel electrophoresis, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel, et al. (eds), 1989, Current Protocols in Molecular Biology, Vol. 1, Green Publishing Associates, Inc., and John Wiley & Sons, Inc., New York) and are commonly known in the art. Hybridization to filter-bound sequences under moderately stringent conditions may, for example, be performed in 0.5 M NaHPO4, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO4, 7% SDS, 1 mM EDTA at 65° C., and washing in 0.1×SSC/0.1% SDS at 68° C. (see Ausubel, et al. (eds), 1989, supra). In other examples of hybridization, a nitrocellulose filter can be incubated overnight at 65° C. with a labeled probe specific to one or the other two alleles in a solution containing 50% formamide, high salt (5×SSC or 5×SSPE), 5×Denhardt's solution, 1% SDS, and 100 μg/ml denatured carrier DNA (i.e. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). The selected temperature is based on the melting temperature (Tm) of the DNA hybrid (Sambrook et al. 1989, supra). Generally, stringent conditions are selected to be about 5° C. lower than the thermal melting point for the specific sequence at a defined ionic strength and pH.

In another embodiment, the methods described herein further comprises obtaining or collecting a biological sample from a subject. In various embodiments, the sample can be from any source that contains biological material suitable for the detection of the mutation(s), such as genomic DNA, RNA (cDNA), and/or proteins, for example a tissue or cell sample from the subject (blood cells, immune cells (e.g., lymphocytes), etc. that comprises leukemic cells (AML cells). The sample may be subjected to cell purification/enrichment techniques to obtain a cell population enriched in a specific cell subpopulation or cell type(s). The sample may be subjected to commonly used isolation and/or purification techniques for enrichment in nucleic acids (genomic DNA, cDNA, mRNA) and/or proteins. Accordingly, in an embodiment, the method may be performed on an isolated nucleic acid and/or protein sample, such as isolated genomic DNA. The biological sample may be collected using any methods for collection of biological fluid, tissue or cell sample, such as venous puncture for collection of blood cell samples.

In an embodiment, the method described herein may be combined with other markers, assays, methods and criteria for characterizing, diagnosing or prognosing AMLs/EVI1-r AMLs, i.e. chromosomal rearrangement, monosomy 7, etc.

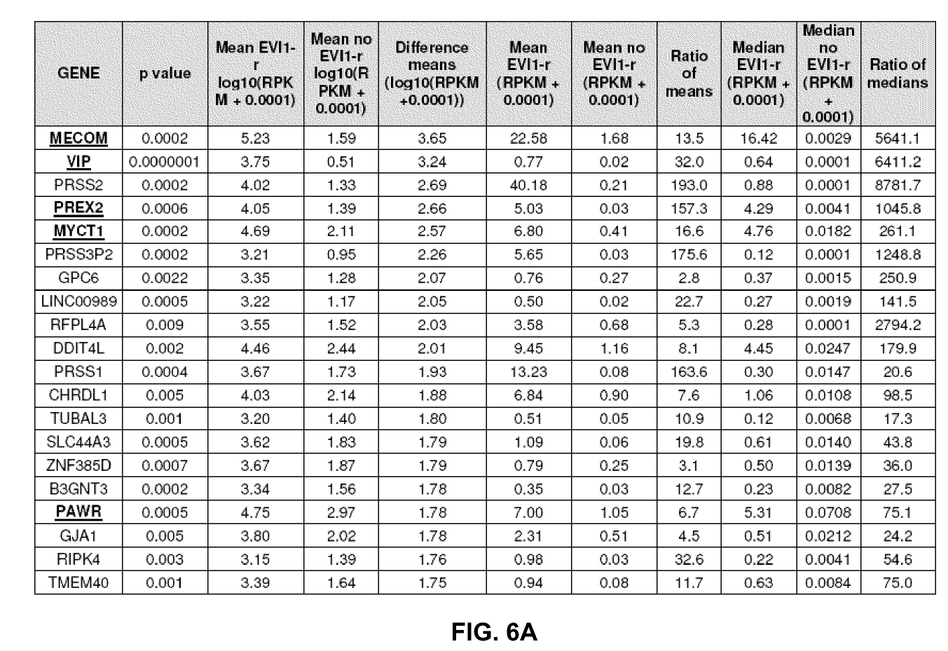

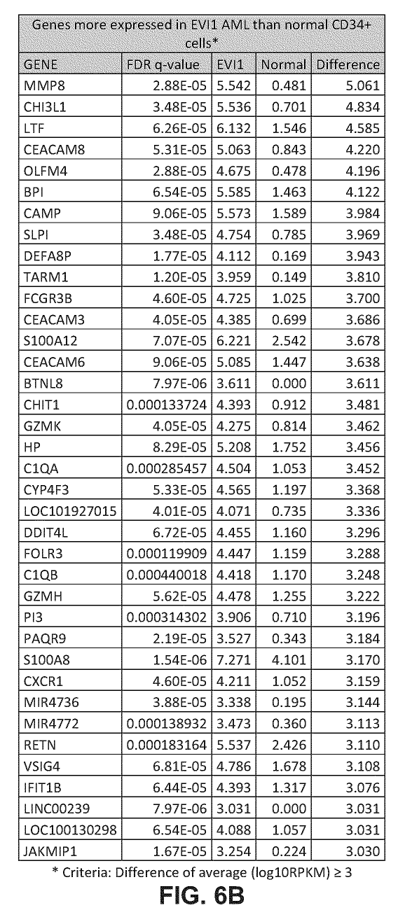

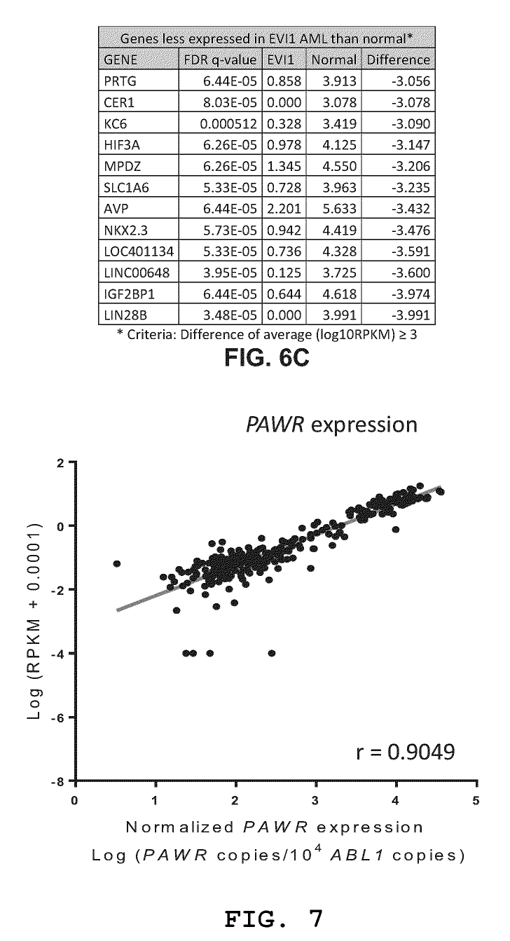

In another aspect, the present invention relates to a method for determining the likelihood that a subject suffers from EVI1-rearranged acute myeloid leukemia (EVI1-r AML), said method comprising: determining/measuring the level of expression of at least one of the genes listed in Table 2, FIG. 6B or 6C in a leukemia cell sample from said subject: comparing said level of expression to a control/reference level of expression (e.g., expression in a non-EVI1-r AML leukemia sample and/or a normal CD34+ cell sample) and determining the likelihood that said subject suffers from EVI1-r AML based on said comparison, wherein a differential expression of said at least one gene in said sample relative to said control sample is indicative that said subject has a high likelihood of suffering from EVI1-r AML.

In another aspect, the present invention relates to a method for determining the likelihood that a subject suffers from EVI1-rearranged acute myeloid leukemia (EVI1-r AML), said method comprising: determining/measuring the level of expression of at least one of the genes listed in Table 2 in a leukemia cell sample from said subject: wherein a higher expression of said at least one gene in said sample relative to a control non-EVI1-r AML sample is indicative that said subject has a high likelihood of suffering from EVI1-r AML.

In another aspect, the present invention relates to a method for determining the likelihood that an AML sample is an EVI1-r AML sample, said method comprising: determining/measuring the level of expression of at least one of the genes listed in Table 2 in said AML sample: wherein a higher expression of said at least one gene in said sample relative to a control non-EVI1-r AML sample is indicative that said sample has a high likelihood of being an EVI1-r AML sample, and wherein a similar or lower expression of said at least one gene in said sample relative to a control non-EVI1-r AML sample is indicative that said sample has a low likelihood of being an EVI1-r AML sample.

The present invention encompasses the determination of the level of expression of any gene or any combination/sub-combination of the genes defined herein (e.g., those depicted in Table 2 and FIGS. 6A-6C), for example the determination of the level of expression of a single gene, or of 2, 3, 4, 5 or more of the genes defined herein.

The Genbank or RefSeq accession numbers or sequence identifiers corresponding to the genes/transcripts exhibiting altered expression in EVI1-r AMLs relative to non-EVI1-r AMLs are indicated in Table 2 below.

TABLE 2

| | Genbank or RefSeq accession | | |
|---|---|---|---|
| Gene | Protein/cDNA/genomic (SEQ ID | Gene | Genbank or RefSeq accession |
| name | NO:) | name | Protein/cDNA/genomic |
| MECOM | NP_001098547.3/NM_001105077/ | ZNF385D | NP_078973.1/NM_024697.2/ |
| isoform 1d | Gene ID: 2122 (SEQ ID NO: 19-20) | | Gene ID: 79750 (SEQ ID NO: 39-40) |
| MECOM | NP_001157472.1/NM_001164000/ | SLC44A3 | NP_001107578.1/NM_001114106.2/ |
| isoform 1a | Gene ID: 2122 (SEQ ID NO: 21-22) | | Gene ID: 126969 (SEQ ID NO: 41-42) |
| VIP | NP_003372.1/NM_003381.3/ | GJA1 | NP_000156.1/NM_000165.3/ |
| | Gene ID: 743222 (SEQ ID NO: 23-24) | | NG_008308.1 (SEQ ID NO: 43-44) |
| PREX2 | NP_079146.2/NM_024870.2/ | CHRDL1 | NP_001137453.1/NM_001143981.1/ |
| | Gene ID: 80243 (SEQ ID NO: 25-26) | | NG_012816.1 (SEQ ID NO: 45-46) |
| MYCT1 | NP_079383.2/NM_025107.2/ | PRSS3P2 | NR_001296.3/Gene ID: 154754 |
| | Gene ID: 80177 (SEQ ID NO: 27-28) | | (SEQ ID NO: 47) |
| PAWR | NP_002574.2/NM_002583.2/ | DDIT4L | NP_660287.1/NM_145244.3/ |
| | Gene ID: 5074 (SEQ ID NO: 7-8) | | Gene ID: 115265 (SEQ ID NO: 48-49) |
| PRSS1 | NP_002760.1/NM_002769.4/ | GPC6 | NP_005699.1/NM_005708.3/Gene |
| | NG_008307.2 (SEQ ID NO: 29-30) | | ID: 10082 (SEQ ID NO: 50-51) |
| PRSS2 | NP_002761.1/NM_002770.2/ | LINC00989 | NR_038826.1/Gene ID: 100506035 |
| | NG_008322.1 (SEQ ID NO: 31-32) | | (SEQ ID NO: 52) |
| RFPL4A | NP_001138486.1/ | TUBAL3 | NP_079079.1/NM_024803.2/Gene |
| | NM_001145014.1/Gene ID: 342931 | | ID: 79861 (SEQ ID NO: 53-54) |
| | (SEQ ID NO: 33-34) | | |
| B3GNT3 | NP_055071.2/NM_014256.3/Gene | RIPK4 | NP_065690.2/NM_020639.2/Gene |
| | ID: 10331 (SEQ ID NO: 35-36) | | ID: 54101 (SEQ ID NO: 55-56) |
| TMEM40 | NP_001271335.1/ | MECOM | SEQ ID NO: 9 |
| | NM_001284406.1/Gene ID: 55287 | isoform 5 | |
| | (SEQ ID NO: 37-38) | | |
| MECOM | SEQ ID NO: 10 | MECOM | SEQ ID NO: 11 |
| isoform 16 | | isoform 20 | |
| MECOM | SEQ ID NO: 12 | MECOM | SEQ ID NO: 13 |
| isoform 21 | | isoform 22 | |
| MECOM | SEQ ID NO: 14 | MECOM | SEQ ID NO: 15 |
| isoform 28 | | isoform 29 | |
| MYCT1 | SEQ ID NO: 16 | LRBA | SEQ ID NO: 17 |
| isoform 6 | | isoform 3 | |
| LRBA | SEQ ID NO: 18 | | |
| isoform 6 | | | |

In another aspect, the present invention relates to a method for determining the likelihood that a subject suffers from EVI1-rearranged acute myeloid leukemia (EVI1-r AML), said method comprising: determining the level of expression of at least one of the genes listed in FIG. 6B and/or FIG. 6C in a leukemia cell sample from said subject: wherein a higher expression of said at least one of genes of FIG. 6B and/or a lower expression of said at least one of genes of FIG. 6C, in said sample relative to a control CD34+ cell sample, is indicative that said subject has a high likelihood of suffering from EVI1-r AML.

The determination of the expression of the one or more genes or encoded gene products (e.g., mRNA, protein) listed above may be performed using any known methods to detect nucleic acids or proteins. In embodiments, the expression is compared to a control or reference level (e.g., the level obtained a sample from a non-EVI1-r AML sample, and/or a cell sample enriched in CD34⁺ cells) to assess the subject's likelihood of suffering from EVI1-r AML, or the likelihood that the AML sample is an EVI1-r AML sample.

In another aspect, the present invention relates to a method for the disease prognosis of a subject suffering from acute myeloid leukemia (AML), said method comprising: measuring the level of expression of one or more of the genes listed in Table 2 in a biological sample from said subject; and comparing said level of expression to a threshold reference level, wherein a level of expression that is above said threshold reference level is indicative of a poor disease prognosis.

In another aspect, the present invention relates to a method for the disease prognosis of a subject suffering from acute myeloid leukemia (AML), said method comprising: measuring the level of expression of PAWR in a biological sample from said subject; and comparing said level of expression to a threshold reference level, wherein a level of expression that is above said threshold reference level is indicative of a poor disease prognosis.

In an embodiment, the above-mentioned method if used for the prognosis of intermediate-risk AML, which includes for example normal karyotype (NK) AML, NUP98-NSD1 fusion in AML with normal karyotype (NK), trisomy 8 alone AML, intermediate abnormal karyotype AML or certain types of AML with MLL translocations such as t(9;11)/MLL-MLLT3, and in a further embodiment intermediate-risk FLT3-ITD negative AML. The term “intermediate-risk AML” (also referred to as “intermediate-risk cytogenetic subclass of AML”) is commonly used to refer to AML without favorable and particular unfavorable cytogenetic aberrations (i.e. “uninformative” cytogenetic aberrations), and account for a significant proportion (approximately 55%) of AML patients.

As used herein, the term “prognosis” refers to the forecast of the probable outcome or course of AML; the patient's chance of recovery or survival. Accordingly, a less favorable, negative or poor prognosis is defined by a lower post-treatment survival term or survival rate. Conversely, a positive, favorable, or good prognosis is defined by an elevated post-treatment survival term or survival rate. Survival is usually calculated as an average number of months (or years) that 50% of patients survive, or the percentage of patients that are alive after 1, 2, 3, 4, 5, 10 years, etc. Prognosis is important for treatment decisions because patients with a good prognosis are usually offered less invasive/aggressive treatments (e.g., standard chemotherapy), while patients with poor prognosis are usually offered more aggressive treatment, such as more extensive chemotherapy drugs, stem cell/bone marrow transplantation, and/or any other aggressive treatment. In an embodiment, the poor disease prognosis comprises poor overall survival, for example a low likelihood (e.g., less than about 50, 40, 30, 20, or 10%) of survival over a period of 1, 2, 3, 4 or 5 years.

The levels of nucleic acids corresponding to the above-mentioned genes (e.g., transcripts) can then be evaluated according to the methods disclosed below, e.g., with or without the use of nucleic acid amplification methods. In some embodiments, nucleic acid amplification methods can be used to detect the level of expression of the one or more genes. For example, the oligonucleotide primers and probes may be used in amplification and detection methods that use nucleic acid substrates isolated by any of a variety of well-known and established methodologies (e.g., Sambrook et al., Molecular Cloning, A laboratory Manual, pp. 7.37-7.57 (2nd ed., 1989); Lin et al., in Diagnostic Molecular Microbiology, Principles and Applications, pp. 605-16 (Persing et al., eds. (1993); Ausubel et al., Current Protocols in Molecular Biology (2001 and later updates thereto)). Methods for amplifying nucleic acids include, but are not limited to, for example the polymerase chain reaction (PCR) and reverse transcription PCR (RT-PCR) (see e.g., U.S. Pat. Nos. 4,683,195; 4,683,202; 4,800,159; 4,965,188), ligase chain reaction (LCR) (see, e.g., Weiss, Science 254: 1292-93 (1991)), strand displacement amplification (SDA) (see e.g., Walker et al., Proc. Natl. Acad. Sci. USA 89:392-396 (1992); U.S. Pat. Nos. 5,270,184 and 5,455,166), Thermophilic SDA (tSDA) (see e.g., European Pat. No. 0 684 315) and methods described in U.S. Pat. No. 5,130,238; Lizardi et al., BioTechnol. 6:1197-1202 (1988); Kwoh et al., Proc. Natl. Acad. Sci. USA 86:1173-77 (1989); Guatelli et al., Proc. Natl. Acad. Sci. USA 87:1874-78 (1990); U.S. Pat. Nos. 5,480,784; 5,399,491; U.S. Publication No. 2006/46265. The methods include the use of Transcription Mediated Amplification (TMA), which employs an RNA polymerase to produce multiple RNA transcripts of a target region (see, e.g., U.S. Pat. Nos. 5,480,784; 5,399,491 and U.S. Publication No. 2006/46265). The levels of nucleic acids may also be measured by “Next Generation Sequencing” (NGS) methods such as RNA sequencing.

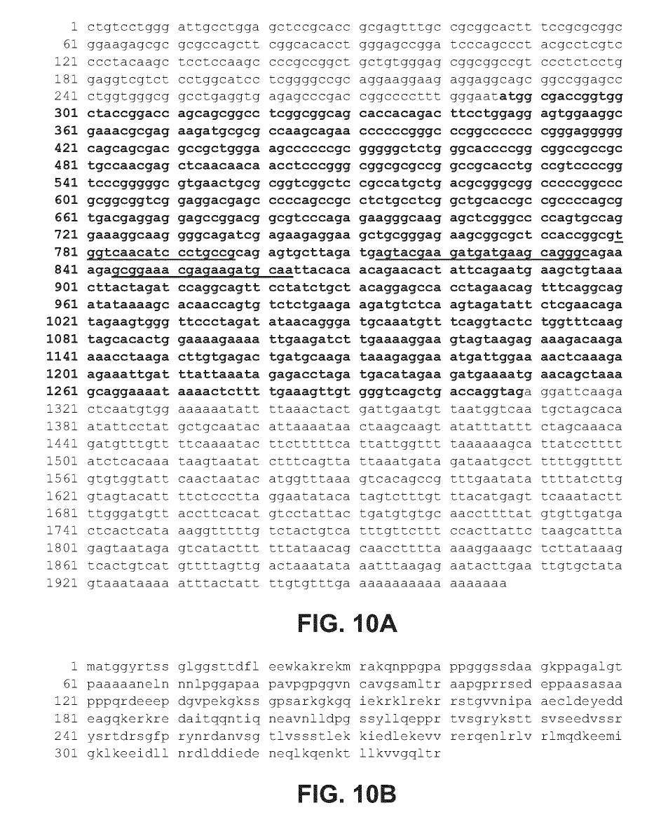

In an embodiment, the above-mentioned method comprises a step of amplification. In an embodiment, the level of expression of PAWR is measured and the method comprises amplifying a PAWR nucleic acid using a suitable pair of primers. Suitable pairs of primers may be designed based on the nucleotide sequence of PAWR (FIG. 10A, SEQ ID NO: 7). In an embodiment, each of the primer comprises from about 7-8 to about 100, 90, 80, 70, 60 or 50 nucleotides, in further embodiments from about 10 to about 50, 45 or 40 nucleotides, from about 10 to about 35 nucleotides, from about 10 to about 35, 34, 33, 32, 31 or 30 nucleotides, from about 15 to about 25 nucleotides or from about 16 to about 24 nucleotides. In an embodiment, each of the primer comprises about 10, 11, 12, 13, 14, 15, 16, 17, 18, 19, 20, 21, 22, 23, 24, 25, 26, 27, 28, 29 or 30 nucleotides. In an embodiment, each of the primer comprises a sequence corresponding to at least 10 nucleotides (e.g., contiguous) of SEQ ID NO: 7), or its complement.

In an embodiment, the pair of primers comprises a first primer comprising at least 10 nucleotides of the sequence 5′-TGGTCAACATCCCTGCCG-3′ (SEQ ID NO: 3) and/or a second primer comprising at least 10 nucleotides of the sequence 5′-TTGCATCTTCTCGTTTCCGC-3′ (SEQ ID NO: 4). In an embodiment, the first primer comprises at least 11, 12, 13, 14, 15, 16, 17 or 18 nucleotides of the sequence 5′-TGGTCAACATCCCTGCCG-3′ (SEQ ID NO: 3). In a further embodiment, the first primer comprises, or consists of, the sequence 5′-TGGTCAACATCCCTGCCG-3′ (SEQ ID NO: 3). In an embodiment, the second primer comprises at least 11, 12, 13, 14, 15, 16, 17, 18, 19 or 20 nucleotides of the sequence 5′-TTGCATCTTCTCGTTTCCGC-3′ (SEQ ID NO: 4). In a further embodiment, the second primer comprises, or consists of, the sequence 5′-TTGCATCTTCTCGTTTCCGC-3′ (SEQ ID NO: 4).

The nucleic acid or amplification product may be detected or quantified by hybridizing a probe (e.g., a labeled probe) to a portion of the nucleic acid or amplified product. The probe may be labelled with a detectable group that may be, for example, a fluorescent moiety, chemiluminescent moiety, radioisotope, biotin, avidin, enzyme, enzyme substrate, or other reactive group. Other well-known detection techniques include, for example, gel filtration, gel electrophoresis and visualization of the amplicons, and High Performance Liquid Chromatography (HPLC). In certain embodiments, for example using real-time TMA or real-time PCR, the level of amplified product is detected as the product accumulates.

In an embodiment, the above-mentioned method comprises a step of detection or quantification with a probe. In an embodiment, the level of expression of PAWR is measured and the method comprises detecting or quantifying the nucleic acid or amplified product with a probe. Suitable probes may be designed based on the nucleotide sequence of PAWR (FIG. 10A, SEQ ID NO: 7) In an embodiment, the probe comprises from about 7-8 to about 100, 90, 80, 70, 60 or 50 nucleotides, in further embodiments from about 10 to about 50, 45 or 40 nucleotides, from about 10 to about 35 nucleotides, from about 10 to about 35, 34, 33, 32, 31 or 30 nucleotides, from about 15 to about 25 nucleotides or from about 16 to about 24 nucleotides. In an embodiment, the probe comprises about 10, 11, 12, 13, 14, 15, 16, 17, 18, 19, 20, 21, 22, 23, 24, 25, 26, 27, 28, 29 or 30 nucleotides. In an embodiment, the probe comprises a sequence corresponding to at least 10 nucleotides (e.g., contiguous) of SEQ ID NO: 7, or its complement.

In an embodiment, the probe comprises at least about 10 nucleotides of the sequence 5′-AGTACGAAGATGATGAAGCAGGGC-3′ (SEQ ID NO: 6). In an embodiment, the probe comprises at least about 11, 12, 13, 14, 15, 16, 17, 18, 19, 20, 21, 22, 23 or 24 nucleotides of the sequence 5′-AGTACGAAGATGATGAAGCAGGGC-3′ (SEQ ID NO: 6). In an embodiment, the probe comprises, or consists of, the sequence 5′-AGTACGAAGATGATGAAGCAGGGC-3′ (SEQ ID NO: 6).

In an embodiment, the above-mentioned method comprises a step of normalizing the gene expression levels, i.e. normalization of the measured levels of the above-noted genes against a stably expressed control gene (or housekeeping gene) to facilitate the comparison between different samples. “Normalizing” or “normalization” as used herein refers to the correction of raw gene expression values/data between different samples for sample to sample variations, to take into account differences in “extrinsic” parameters such as cellular input, nucleic acid (RNA) or protein quality, efficiency of reverse transcription (RT), amplification, labeling, purification, etc., i.e. differences not due to actual “intrinsic” variations in gene expression by the cells in the samples. Such normalization is performed by correcting the raw gene expression values/data for a test gene (or gene of interest) based on the gene expression values/data measured for one or more “housekeeping” or “control” genes, i.e. whose expressions are known to be constant (i.e. to show relatively low variability) between the cells of different tissues and under different experimental conditions. Thus, in an embodiment, the above-mentioned method further comprises measuring the level of expression of a housekeeping gene in the biological sample. Suitable housekeeping genes are known in the art and several examples are described in WO 2014/134728, including those depicted in Table 3 below.

TABLE 3
Examples of housekeeping genes

| Gene | GenBank Accession # |
|---|---|
| ABI1 | NM_001012750, NM_001012751, NM_001012752, NM_001178116, |
| | NM_001178119, NM_001178120, NM_001178121, NM_001178122, |
| | NM_001178123, NM_001178124, NM_001178125, NM_005470 |
| ACIN1 | NM_001164814, NM_001164815, NM_001164816, NM_001164817, |
| | NM_014977 |
| ACP1 | NM_001040649, NM_004300, NM_007099 |
| ADAR | NM_001025107, NM_001111, NM_001193495 |
| ADD1 | NM_001119, NM_014189, NM_014190, NM_176801 |
| ANAPC5 | NM_001137559, NM_016237 |
| ARF1 | NM_001024226, NM_001024227, NM_001024228, NM_001658 |
| ATP5B | NM_001686 |
| ATP6V1G1 | NM_004888 |
| ATXN2L | NM_007245, NM_017492, NM_145714, NM_148414, NM_148415, NM_148416 |
| AUP1 | NM_181575 |
| C1orf144 | NM_001114600, NM_015609 |
| C20orf43 | NM_016407 |
| C6orf62 | NM_030939 |
| CAPRIN1 | NM_005898, NM_203364 |
| CASC3 | NM_007359 |
| CCNI | NM_006835 |
| CDC37 | NM_007065 |
| CDV3 | NM_001134422, NM_001134423, NM_017548 |
| CMPK1 | NM_001136140, NM_016308 |
| CMTM3 | NM_144601, NM_181553 |
| COPB1 | NM_001144061, NM_001144062, NM_016451 |
| COPS5 | NM_006837 |
| CS | NM_004077 |
| CSDE1 | NM_001007553, NM_001130523, NM_001242891, NM_001242892, |
| | NM_001242893, NM_007158 |
| DAP3 | NM_001199849, NM_001199850, NM_001199851, NM_004632, NM_033657 |
| DCAF8 | NM_015726 |
| DDX5 | NM_004396 |
| DLST | NM_001933 |
| DNAJC7 | NM_001144766, NM_003315 |
| DOCK2 | NM_004946 |
| E2F4 | NM_001950 |
| EIF3I | NM_003757 |
| EIF4H | NM_022170, NM_031992 |
| EWSR1 | NM_001163285, NM_001163286, NM_001163287, NM_005243, NM_013986 |
| FAM32A | NM_014077 |
| GABARAPL2 | NM_007285 |
| GNB1 | NM_002074, NM_001282538, NM_001282539 |
| GORASP2 | NM_001201428, NM_015530 |
| GTF2F1 | NM_002096 |
| HDAC3 | NM_003883 |
| HNRNPA2B1 | NM_002137, NM_031243 |
| HNRNPC | NM_001077442, NM_001077443, NM_004500, NM_031314 |
| HNRNPD | NM_002138, NM_031369, NM_031370 |
| HNRNPH3 | NM_012207, NM_021644 |
| HNRNPK | NM_002140, NM_031262, NM_031263 |
| HNRNPL | NM_001005335, NM_001533 |
| HNRNPU | NM_004501, NM_031844 |
| HNRNPUL1 | NM_007040 |
| IDH3B | NM_001258384, NM_006899, NM_174855, NM_174856 |
| IK | NM_006083 |
| KARS | NM_001130089, NM_005548 |
| KHDRBS1 | NM_006559 |
| LSM14A | NM_001114093, NM_015578 |
| MAPRE1 | NM_012325 |
| MARS | NM_004990 |
| MLF2 | NM_005439 |
| MMADHC | NM_015702 |
| MORF4L1 | NM_001265605, NM_006791, NM_206839 |
| MRFAP1 | NM_033296 |
| MRPL9 | NM_031420 |
| MTA2 | NM_004739 |
| MYL12B | NM_001144944, NM_001144945, NM_033546 |
| NOL7 | NM_016167 |
| NRD1 | NM_001101662, NM_001242361, NM_002525 |
| OCIAD1 | NM_001079839, NM_001079840, NM_001079841, NM_001079842, |
| | NM_001168254, NM_017830 |
| PAPOLA | NM_001252006, NM_032632 |
| PCBP2 | NM_001098620, NM_001128911, NM_001128912, NM_001128913, |
| | NM_001128914, NM_005016, NM_031989 |
| POLR2C | NM_032940 |
| PSMA1 | NM_002786, NM_148976 |
| PSMB1 | NM_002793 |
| PSMD2 | NM_002808 |
| PSMD6 | NM_014814, NM_001271780, NM_001271779, NM_001271781 |
| PSMD7 | NM_002811 |
| PSME1 | NM_006263, NM_176783 |
| PSME3 | NM_001267045, NM_005789, NM_176863 |
| PSMF1 | NM_006814, NM_178578 |
| PTPRA | NM_002836, NM_080840, NM_080841 |
| RAB7A | NM_004637 |
| RBM22 | NM_018047 |
| RBM8A | NM_005105 |
| RHOA | NM_001664 |
| RNF114 | NM_018683 |
| RNF7 | NM_001201370, NM_014245, NM_183237 |
| SEC22B | NM_0048925 |
| SEC31A | NM_001077206, NM_001077207, NM_001077208, NM_001191049, |
| | NM_014933, NM_016211 |
| SERP1 | NM_014445 |
| SF3A1 | NM_001005409, NM_005877 |
| SF3B2 | NM_006842 |
| SLC25A3 | NM_002635, NM_005888, NM_213611 |
| SNW1 | NM_012245 |
| SON | NM_032195, NM_138927 |
| SRP14 | NM_003134 |
| SRPR | NM_001177842, NM_003139 |
| SRSF5 | NM_001039465, NM_006925 |
| SRSF9 | NM_003769 |
| SSR2 | NM_003145 |
| STX16 | NM_001001433, NM_001134772, NM_001134773, NM_001204868, |
| | NM_003763 |
| SUMO1 | NM_001005781, NM_001005782, NM_003352 |
| SUMO3 | NM_006936 |
| SUPT6H | NM_003170 |
| TCEB1 | NM_001204857, NM_001204858, NM_001204859, NM_001204860, |
| | NM_001204861, NM_001204862, NM_001204863, NM_001204864, |
| | NM_005648 |
| TH1L | NM_198976 |
| TMED2 | NM_006815 |
| TMEM50A | NM_014313 |
| TRIP12 | NM_004238 |
| U2AF1 | NM_001025203, NM_001025204, NM_006758 |
| UBE2D3 | NM_003340, NM_181886, NM_181887, NM_181888, NM_181889, NM_181890, |
| | NM_181891, NM_181892, NM_181893 |
| UBE2I | NM_003345, NM_194259, NM_194260, NM_194261 |
| UBE2Z | NM_023079 |
| UBQLN1 | NM_013438, NM_053067 |
| USP39 | NM_001256725, NM_001256726, NM_001256728, NM_006590 |
| USP4 | NM_001251877, NM_003363, NM_199443 |
| VCP | NM_007126 |
| VPS4A | NM_013245 |
| XRN2 | NM_012255 |
| YME1L1 | NM_014263, NM_139312 |
| ZC3H11A | NM_001271675, NM_014827 |
| ZNF207 | NM_001032293, NM_001098507, NM_003457 |

Other commonly used housekeeping genes include TBP, YWHAZ, PGK1, LDHA, ALDOA, HPRT1, SDHA, UBC, GAPDH, ACTB, G6PD, VIM, TUBA1A, PFKP, B2M, GUSB, PGAM1 and HMBS.

In a further embodiment, the method further comprises measuring the level of expression of one or more housekeeping genes in a biological sample from the subject. In an embodiment, the level of expression of the housekeeping gene is measured and the method comprises amplifying a housekeeping gene nucleic acid using a suitable pair of primers. In an embodiment, the housekeeping gene used for normalization is ABL1. In another embodiment, the housekeeping gene used for normalization is PSMA1. In an embodiment, the method comprises amplifying an ABL1 nucleic acid using a suitable pair of primers. Suitable pairs of primers may be designed based on the nucleotide sequence of ABL1, which may be found in GenBank Accession No. NM_001012750, NM_001012751, NM_001012752, NM_001178116, NM_001178119, NM_001178120, NM_001178121, NM_001178122, NM_001178123, NM_001178124, NM_001178125 and NM_005470. In an embodiment, the pair of primer comprises a first primer comprising at least 10 nucleotides of the sequence 5′-TGGAGATAACACTCTAAGCATAACTAAAGGT-3′ (SEQ ID NO: 1) and/or a second primer comprising at least 10 nucleotides of the sequence 5′-GATGTAGTTGCTTGGGACCCA-3′ (SEQ ID NO: 2). In an embodiment, the first primer comprises at least 11, 12, 13, 14, 15, 16, 17, 18, 19, 20, 21, 22, 23, 24, 25, 26, 27, 28, 29, 30 or 31 nucleotides of the sequence 5′-TGGAGATAACACTCTAAGCATAACTAAAGGT-3′ (SEQ ID NO: 1). In a further embodiment, the first primer comprises, or consists of the sequence 5′-TGGAGATAACACTCTAAGCATAACTAAAGGT-3′ (SEQ ID NO: 1). In an embodiment, the second primer comprises at least 11, 12, 13, 14, 15, 16, 17, 18, 19, 20 or 21 nucleotides of the sequence 5′-GATGTAGTTGCTTGGGACCCA-3′ (SEQ ID NO: 2). In a further embodiment, the second primer comprises, or consists of, the sequence 5′-GATGTAGTTGCTTGGGACCCA-3′ (SEQ ID NO: 2).

In an embodiment, the above-mentioned method comprises a step of detection or quantification of the housekeeping gene nucleic acid (e.g. ABL1) with a probe. In an embodiment, the housekeeping gene is ABL1 and the probe comprises at least 10 nucleotides of the sequence 5′-CCATTTTTGGTTTGGGCTTCACACCATT-3′ (SEQ ID NO: 5). In an embodiment, the probe comprises at least 11, 12, 13, 14, 15, 16, 17, 18, 19, 20, 21, 22, 23, 24, 25, 26, 27 or 28 nucleotides of the sequence 5′-CCATTTTTGGTTTGGGCTTCACACCATT-3′ (SEQ ID NO: 5). In a further embodiment, the probe comprises, or consists of, the sequence 5′-CCATTTTTGGTTTGGGCTTCACACCATT-3′ (SEQ ID NO: 5).

In an embodiment, one or more of the primers and/or probe is/are detectably labelled, i.e. comprises a detectable label attached thereto. As used herein, the term “detectable label” refers to a moiety emitting a signal (e.g., light) that may be detected using an appropriate detection system. Any suitable detectable label may be used in the method described herein. Detectable labels include, for example, enzyme or enzyme substrates, reactive groups, chromophores such as dyes or colored particles, luminescent moieties including bioluminescent, phosphorescent, or chemiluminescent moieties, and fluorescent moieties. In an embodiment, the detectable label is a fluorescent moiety. Fluorophores that are commonly used include, but are not limited to, fluorescein, 5-carboxyfluorescein (FAM), 2′7′-dimethoxy-4′5′-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), and 5-(2′-aminoethyl)aminonaphthalene-I-sulfonic acid (EDANS). The fluorophore may be any fluorophore known in the art, including, but not limited to: FAM, TET, HEX, Cy3, TMR, ROX, Texas Red®, LC red 640, Cy5, and LC red 705. Fluorophores for use in the methods and compositions provided herein may be obtained commercially, for example, from Biosearch Technologies (Novato, Calif.), Life Technologies (Carlsbad, Calif.), GE Healthcare (Piscataway N.J.), Integrated DNA Technologies (Coralville, Iowa) and Roche Applied Science (Indianapolis, Ind.). In some embodiments, the fluorophore is chosen to be usable with a specific detector, such as a specific spectrophotometric thermal cycler, depending on the light source of the instrument. In some embodiments, if the assay is designed for the detection of two or more target nucleic acids (multiplex assays), for example PAWR and one or more additional prognostic markers, two or more different fluorophores may be chosen with absorption and emission wavelengths that are well separated from each other (i.e., have minimal spectral overlap). In some embodiments, the fluorophore is chosen to work well with one or more specific quenchers. A representative example of a suitable combination of fluorescent label and quenchers is FAM/ZEN/IBFQ, which comprises the fluorescent FAM (excitation max.=494 nm, emission max.=520 nm), the ZEN™ quencher (non-abbreviation; absorption max 532 nm), and the Iowa black fluorescein quencher (IBFQ, absorption max=531 nm) (Integrated DNA Technologies®). Covalent attachment of detectable label and/or quencher to primer and/or probe can be accomplished according to standard methodology well known in the art as discussed, for example in Sambrook et al. (Molecular Cloning: A Laboratory Manual, Cold Spring Harbor, N.Y., 2001), Ausubel et al. (In Current Protocols in Molecular Biology, John Wiley & Sons, New York, 1998), Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19:3019 (1991) (3′ sulfhydryl); Giusti et al., PCR Methods and Applications, 2:223-227 (1993) and Fung et al, U.S. Pat. No. 4,757,141 (5′ phosphoamino group via Aminolink™ II available from Applied Biosystems®, Foster City, Calif.); Stabinsky, U.S. Pat. No. 4,739,044 (3′ aminoalkylphosphoryl group); Agrawal et al., Tetrahedron Letters, 31:1543-1546 (1990) (attachment via phosphoramidate linkages); Sproat et al., Nucleic Acids Research, 15:4837 (1987) (5′ mercapto group); Nelson et al., Nucleic Acids Research, 17:7187-7194 (1989) (3′ amino group); and the like.

In another embodiment, the expression of the one or more genes or encoded gene products is measured at the protein level. Methods to measure the amount/level of proteins are well known in the art. Protein levels may be detected directly using a ligand binding specifically to the protein, such as an antibody or a fragment thereof. In embodiments, such a binding molecule or reagent (e.g., antibody) is labeled/conjugated, e.g., radio-labeled, chromophore-labeled, fluorophore-labeled, or enzyme-labeled to facilitate detection and quantification of the complex (direct detection). Alternatively, protein levels may be detected indirectly, using a binding molecule or reagent, followed by the detection of the [protein/binding molecule or reagent] complex using a second ligand (or second binding molecule) specifically recognizing the binding molecule or reagent (indirect detection). Such a second ligand may be radio-labeled, chromophore-labeled, fluorophore-labeled, or enzyme-labeled to facilitate detection and quantification of the complex. Enzymes used for labeling antibodies for immunoassays are known in the art, and the most widely used are horseradish peroxidase (HRP) and alkaline phosphatase (AP). Examples of binding molecules or reagents include antibodies (monoclonal or polyclonal), natural or synthetic ligands, and the like.

Examples of methods to measure the amount/level of protein in a sample include, but are not limited to: Western blot, immunoblot, enzyme-linked immunosorbent assay (ELISA), “sandwich” immunoassays, radioimmunoassay (RIA), immunoprecipitation, surface plasmon resonance (SPR), chemiluminescence, fluorescent polarization, phosphorescence, immunohistochemical (IHC) analysis, matrix-assisted laser desorption/ionization time-of-flight (MALDI-TOF) mass spectrometry, microcytometry, microarray, antibody array, microscopy (e.g., electron microscopy), flow cytometry, proteomic-based assays, and assays based on a property or activity of the protein including but not limited to ligand binding or interaction with other protein partners, enzymatic activity, fluorescence. For example, if the protein of interest is a kinase known to phosphorylate of given target, the level or activity of the protein of interest may be determined by the measuring the level of phosphorylation of the target in the presence of the test compound. If the protein of interest is a transcription factor known to induce the expression of one or more given target gene(s), the level or activity of the protein of interest may be determined by the measuring the level of expression of the target gene(s).

In an embodiment, the reference or control level of expression and/or activity is a level measured in one or more non-cancerous (non-AML) cell samples (e.g., normal hematopoietic stem cell sample, normal CD34⁺ cell sample, etc.) or a non-EVI1-r AML sample (an AML sample from another AML subtype or a mixture of other AML subtypes).

“Control level” or “reference level” or “standard level” are used interchangeably herein and broadly refers to a separate baseline level measured in a comparable “control” sample, which is generally from a subject not suffering from the disease (EVI1-r AML), for example an AML sample from another AML subtype (or a mixture of other AML subtypes) or a samples enriched in CD34⁺ cells from a subject not suffering from AML). The corresponding control level may be a level corresponding to an average or median level calculated based of the levels measured in several reference or control subjects (e.g., a pre-determined or established standard level). The control level may be a pre-determined “cut-off” value recognized in the art or established based on levels measured in samples from one or a group of control subjects. For example, the “threshold reference level” of may be a the level corresponding the minimal level of PAWR expression (cut-off) that permits to distinguish in a statistically significant manner AML patients having a poor disease prognosis from those not having a poor prognosis, which may be determined using samples from AML patients with different disease outcomes, for example. Alternatively, the “threshold reference level” of may be a the level corresponding the level of PAWR expression (cut-off) that permits to best or optimally distinguish in a statistically significant manner AML patients having a poor disease prognosis from those not having a poor prognosis. The corresponding reference/control level may be adjusted or normalized for age, gender, race, or other parameters. The “control level” can thus be a single number/value, equally applicable to every patient individually, or the control level can vary, according to specific subpopulations of patients. Thus, for example, older men might have a different control level than younger men, and women might have a different control level than men. The predetermined standard level can be arranged, for example, where a tested population is divided equally (or unequally) into groups, such as a low-risk group, a medium-risk group and/or a high-risk group or into quadrants or quintiles, the lowest quadrant or quintile being individuals with the lowest risk (i.e., lowest level of expression of the one or more genes) and the highest quadrant or quintile being individuals with the highest risk (i.e., highest level of expression of the one or more genes). It will also be understood that the control levels according to the invention may be, in addition to predetermined levels or standards, levels measured in other samples (e.g. from healthy/normal subjects, or cancer patients) tested in parallel with the experimental sample. The reference or control levels may correspond to normalized levels, i.e. reference or control values subjected to normalization based on the expression of a housekeeping gene. In an embodiment, the threshold reference level corresponds to a normalized (log₁₀) value of PAWR copy number of about ≧800, 850, 900, 950 or 1000 per 10⁴ ABL1 copy number, as described herein. The skilled person would understand that a corresponding threshold reference level, which would define a similar threshold value for PAWR expression levels, may be calculated based, for example, on the expression of another housekeeping gene or using another method of calculation.

“Higher expression” or “higher level of expression” as used herein refers to (i) higher expression of the one or more of the above-mentioned genes (protein and/or mRNA) in one or more given cells present in the sample (relative to the control) and/or (ii) higher amount of cells expressing the one or more genes in the sample (relative to the control). “Lower expression” or “lower level of expression” as used herein refers to (i) lower expression of the one or more genes (protein and/or mRNA) in one or more given cells present in the sample (relative to the control) and/or (ii) lower amount of cells expressing the one or more genes in the sample (relative to the control). In an embodiment, higher or lower refers to a level of expression that is above or below the control level (e.g., the predetermined cut-off value). In another embodiment, higher or lower refers to a level of expression that is at least one standard deviation above or below the control level (e.g., the predetermined cut-off value) (e.g. that is statistically significant as determined using a suitable statistical analysis), and a “similar expression” or “similar level of expression” refers to a level of expression that is less than one standard deviation above or below the control level (e.g., the predetermined cut-off value) (e.g. that is not statistically significant as determined using a suitable statistical analysis). In embodiments, higher or lower refers to a level of expression that is at least 1.5, 2, 2.5, 3, 4 or 5 standard deviations above or below the control level (e.g., the predetermined cut-off value. In another embodiment, “higher expression” refers to an expression that is at least 10, 20, 30, 40, 45 or 50% higher in the test sample relative to the control level. In an embodiment, “lower expression” refers to an expression that is at least 10, 20, 25, 30, 35, 40, 45, or 50% lower in the test sample relative to the control level. In another embodiment, higher or lower refers to a level of expression that is at least 1.5, 2-, 5-, 10-, 25-, or 50-fold higher or lower in the test sample relative to the control sample.

In another embodiment, the method described herein further comprises obtaining or collecting a biological sample from a subject. In various embodiments, the sample can be from any source that contains biological material suitable for the detection of the nucleic acid(s), such as genomic DNA, RNA (cDNA), and/or proteins, for example a tissue or cell sample from the subject (blood cells, immune cells (e.g., lymphocytes), bone marrow cells, etc. that comprises leukemic cells (AML cells). The sample may be subjected to cell purification/enrichment techniques to obtain a cell population enriched in a specific cell subpopulation or cell type(s). The sample may be subjected to commonly used isolation and/or purification techniques for enrichment in nucleic acids (genomic DNA, cDNA, mRNA) and/or proteins. Accordingly, in an embodiment, the method may be performed on an isolated nucleic acid and/or protein sample, such as cDNA. The biological sample may be collected using any methods for collection of biological fluid, tissue or cell sample, such as venous puncture for collection of blood cell samples. Thus, the term “biological sample comprising leukemic cells” as used herein refers to a crude leukemic cell sample, a sample enriched in certain cells (i.e., that has been subjected to cell purification/enrichment techniques), or isolated nucleic acids (RNA, cDNA) and/or proteins from leukemic cells (subjected or not to nucleic acid amplification). In an embodiment, the biological sample comprising leukemic cells comprises nucleic acids (RNA, cDNA) obtained or isolated from leukemic cells.

In certain embodiments, methods of diagnosis described herein may be at least partly, or wholly, performed in vitro. In a further embodiment, the method is wholly performed in vitro.

In an embodiment, the above-mentioned method further comprises selecting and/or administering a course of therapy or prophylaxis to said subject in accordance with the diagnostic/prognostic result. For example, if it is determined that the subject has a high likelihood of suffering from EVI1-r AML or has a poor AML disease prognosis, a more aggressive or a treatment regimen adapted for treatment of EVI1-r AML or poor prognosis AML may be used, such as for example a more aggressive chemotherapy regimen (e.g., high-dose chemotherapy, longer administration schedule, etc.) and/or stem cell/bone marrow transplantation (e.g., allogeneic transplantation). The method further comprises subjecting the subject to a suitable anti-leukemia therapy (e.g., bone marrow or hematopoietic stem cell transplantation, chemotherapy, etc.) in accordance with the prognostic result.

In another aspect, the present invention provides a method for treating an AML patient having a poor disease prognosis comprising (i) identifying an AML patient having a poor disease prognosis using the methods described herein; and (ii) treating said patient with a suitable treatment regimen.

In another aspect, the present invention provides performing any combinations of the steps/methods described herein on biological samples from subjects for the diagnosis/prognosis of AML or EVI1-r AML, for example detecting one or more of the mutations described herein (FIG. 5A), detecting the levels of expression of one or more genes of Table 2, e.g., PAWR, etc.

In another aspect, the present invention provides an assay mixture for the assessment of AML (e.g., for the diagnosis of EVI1-r AML), the assay mixture comprising: (i) a biological sample from a subject suffering from AML; and (ii) one or more reagents for detecting one or more of the mutations set forth in FIG. 5A in the sample.

In another aspect, the present invention provides a system for the assessment of AML (e.g., for the diagnosis of EVI1-r AML), comprising: a biological sample obtained from an AML patient; and one or more assays to determine the presence of one or more of the mutations set forth in FIG. 5A in the sample.

In another aspect, the present invention provides an assay mixture for the assessment of AML (e.g., for the diagnosis of EVI1-r AML), the assay mixture comprising: (i) a biological sample from a subject suffering from AML; and (ii) one or more reagents for determining/measuring the level of expression of at least one of the genes listed in Table 2 and FIGS. 6A to 6C in the sample.

In another aspect, the present invention provides a system for the assessment of AML (e.g., for the diagnosis of EVI1-r AML), comprising: a biological sample obtained from an AML patient; and one or more assays to determine the level of expression of one or more of the listed in Table 2 and FIGS. 6A to 6C in the sample.

In another aspect, the present invention provides an assay mixture for the assessment of AML (e.g., for the prognosis of AML, including intermediate-risk AML), the assay mixture comprising: (i) a biological sample from a subject suffering from AML; and (ii) one or more reagents for determining/measuring the level of expression of PAWR in the sample. In an embodiment, the assay mixture comprises reagents for determining/measuring the level of expression of at least 1, 2, 3, 4, or 5 additional prognostic markers in the biological sample.

In another aspect, the present invention provides a system for the assessment of AML (e.g., for the prognosis of AML), comprising: a biological sample obtained from an AML patient; and one or more assays to determine the level of expression of PAWR in the sample.

In another aspect, the present invention further provides a kit for the assessment of AML (e.g., for the diagnosis of EVI1-r AML), the kit comprising: (i) one or more reagents for detecting one or more of the mutations set forth in FIG. 5A in a biological sample. In an embodiment, the kit comprises reagents for detecting at least 2, 3, 4, or 5 of the mutations set forth in FIG. 5A in a biological sample.

In another aspect, the present invention further provides a kit for the assessment of AML (e.g., for the diagnosis of EVI1-r AML), the kit comprising: (i) one or more reagents for determining/measuring the level of expression of at least one of the genes listed in Table 2 and FIGS. 6A to 6C in a biological sample. In an embodiment, the kit comprises reagents for detecting the level of expression of at least 2, 3, 4, or 5 of the genes listed in Table 2 and FIGS. 6A to 6C set forth in a biological sample.

In another aspect, the present invention provides a kit for the assessment of AML (e.g., for the prognosis of AML, including intermediate-risk AML), the assay mixture comprising: (i) one or more reagents for determining/measuring the level of expression of PAWR in a biological sample. In an embodiment, the kit comprises reagents for determining/measuring the level of expression of at least 1, 2, 3, 4, or 5 additional prognostic markers in a biological sample.

In an embodiment, the one or more reagents comprise, for example, primer(s), probe(s), antibody(ies), solution(s), buffer(s), nucleic acid amplification reagent(s) (e.g., DNA polymerase, DNA polymerase cofactor, dNTPs), nucleic acid hybridization/detection reagent(s), and/or reagents for detecting antigen-antibody complexes, etc. In an embodiment, the assay mixture or kit comprises one or more pairs of primers for amplifying one or more nucleic acids correspond to the gene(s) depicted in FIG. 5A, Table 2 and/or FIGS. 6A-6C. In an embodiment, the assay mixture or kit comprises one or more probes for detecting one or more nucleic acids correspond to the gene(s) depicted in FIG. 5A, Table 2 and/or FIGS. 6A-6C. In an embodiment, the assay mixture or kit further comprises one or more reagents for determining/measuring the level of expression of at least one normalization/housekeeping gene (e.g., ABL1) in the sample. Examples of suitable pair of primers for amplifying a ABL1 nucleic acid, and of suitable probes for detecting a ABL1 nucleic acid, are described above.

In an embodiment, the assay mixture or kit for the prognosis of AML comprises (i) a pair of primers suitable for amplifying a PAWR nucleic acid in the sample. In an embodiment, the assay mixture or kit for the prognosis of AML comprises (i) a probe suitable for detecting a PAWR nucleic acid in the sample. In another embodiment, the assay mixture or kit for the prognosis of AML comprises (i) a pair of primers suitable for amplifying a PAWR nucleic acid in the sample; and (ii) a probe suitable for detecting a PAWR nucleic acid in the sample. Examples of suitable pair of primers for amplifying a PAWR nucleic acid, and of suitable probes for detecting a PAWR nucleic acid, are described above. In an embodiment, the assay mixture or kit further comprises one or more reagents (e.g., primers and/or probes) for determining/measuring the level of expression of one or more AML prognostic markers in the sample. In an embodiment, the assay mixture or kit comprises reagents (e.g., primers and/or probes) for determining/measuring the level of expression of at least two AML prognostic markers in the sample. In an embodiment, the assay mixture or kit further comprises one or more primers and/or probes for determining/measuring the level of expression of at least one normalization/housekeeping gene (e.g., ABL1) in the sample.

Furthermore, in an embodiment, the kit may be divided into separate packages or compartments containing the respective reagent components explained above.

In addition, such a kit may optionally comprise one or more of the following: (1) instructions for using the reagents for the diagnosis and/or prognosis of AML/EVI1-r AML, or any combination of these applications; (2) one or more containers; and/or (3) appropriate controls/standards. Such a kit can include reagents for collecting a biological sample from a patient and reagents for processing the biological sample. The kits featured herein can also include an instruction sheet describing how to perform the assays for measuring gene expression or detecting of mutation(s). The instruction sheet can also include instructions for how to determine a reference cohort (control patient population), including how to determine expression levels of genes in the reference cohort and how to assemble the expression data to establish a reference for comparison to a test patient. The instruction sheet can also include instructions for assaying gene expression in a test patient and for comparing the expression level with the expression in the reference cohort to subsequently determine the appropriate treatment regimen for the test patient.

Informational material included in the kits can be descriptive, instructional, marketing or other material that relates to the methods described herein and/or the use of the reagents for the methods described herein. For example, the informational material of the kit can contain contact information, e.g., a physical address, email address, website, or telephone number, where a user of the kit can obtain substantive information about performing a gene expression analysis and interpreting the results, particularly as they apply to an AML patient's likelihood of having a poor prognosis/outcome.

The kits featured herein can also contain software necessary to infer a patient's likelihood of having a poor prognosis/outcome from the gene expression or mutation data.

In another aspect, there is provided the use of the kit or assay mixture described herein for prognosis of a subject suffering from AML.

### DESCRIPTION OF ILLUSTRATIVE EMBODIMENTS

The present invention is illustrated in further details by the following non-limiting examples.

### Example 1: Materials and Methods

Specimen Collection, Ethics and Cohort Characteristics.

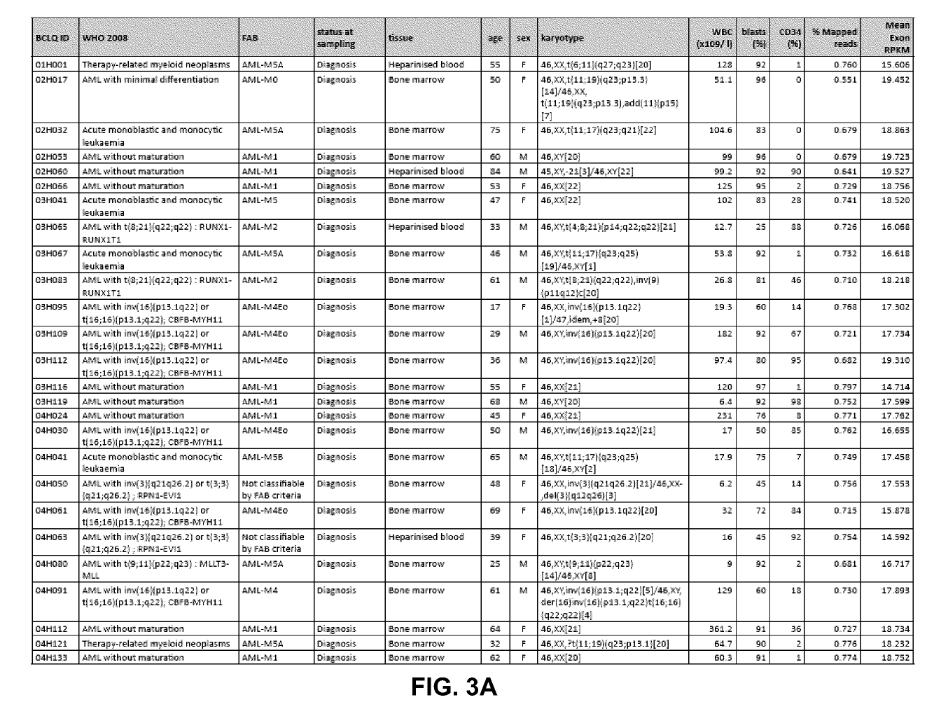

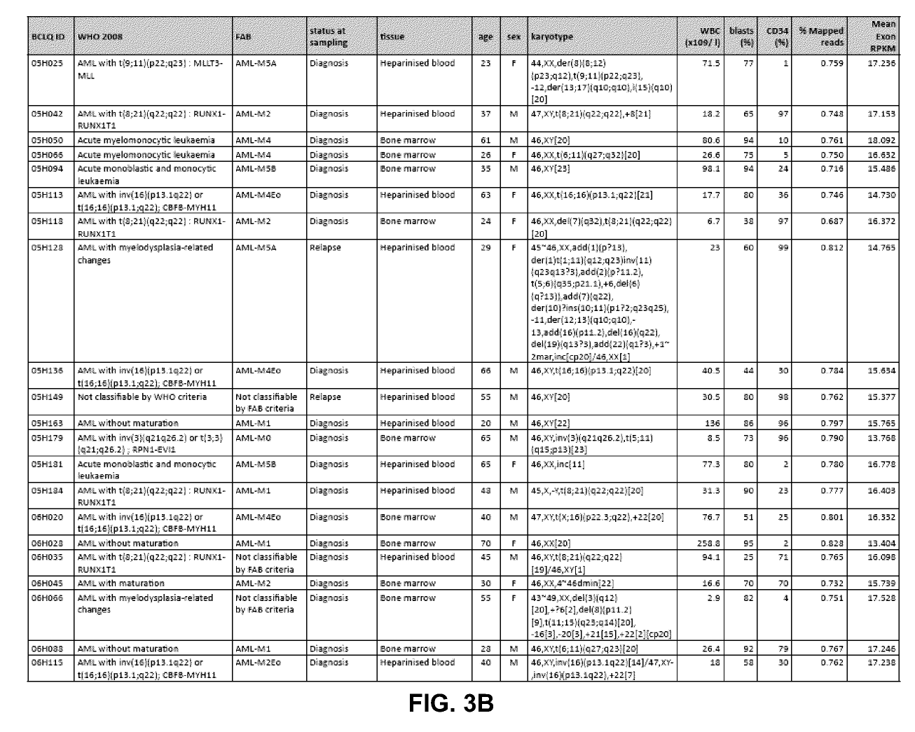

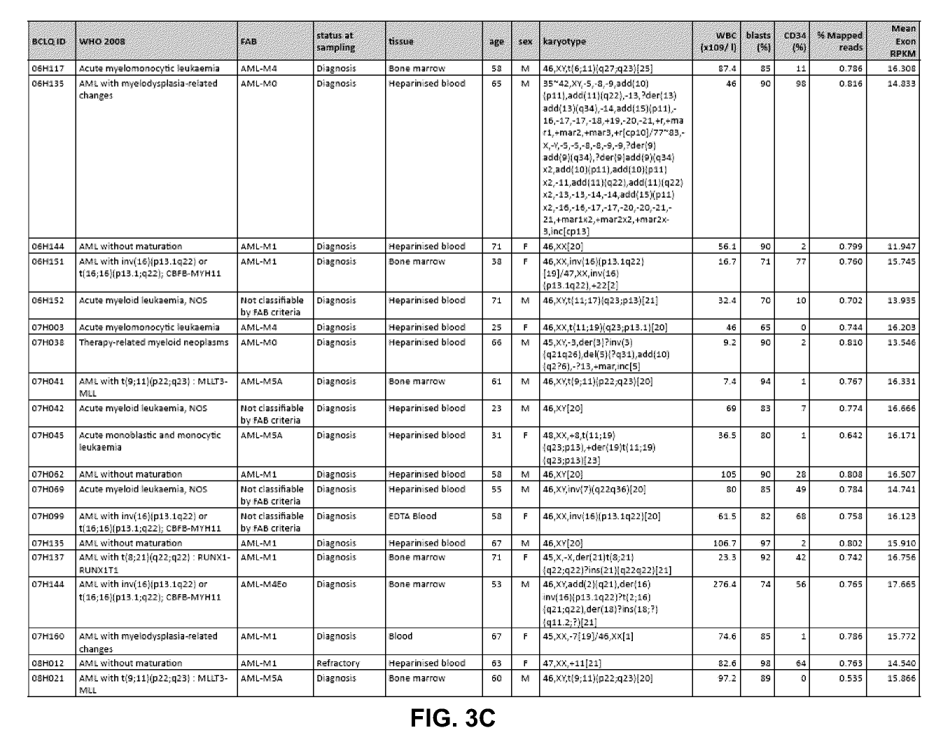

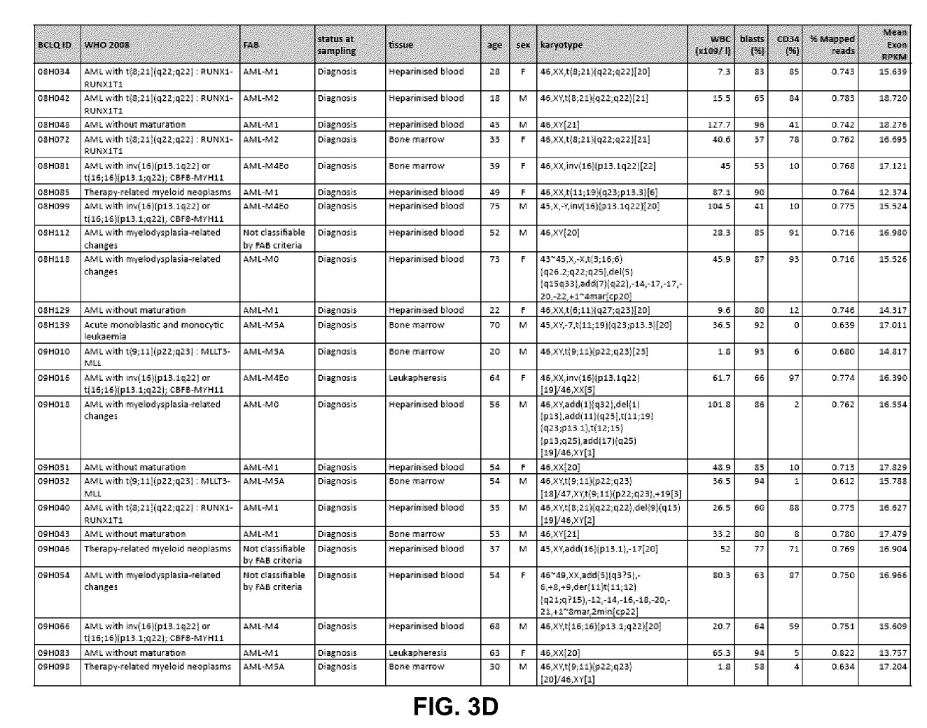

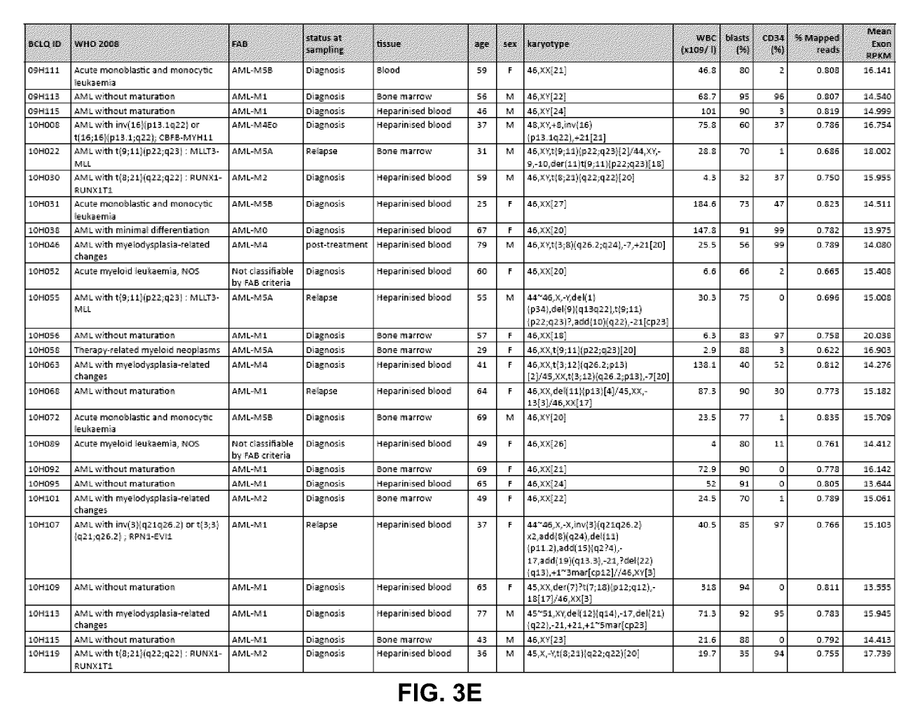

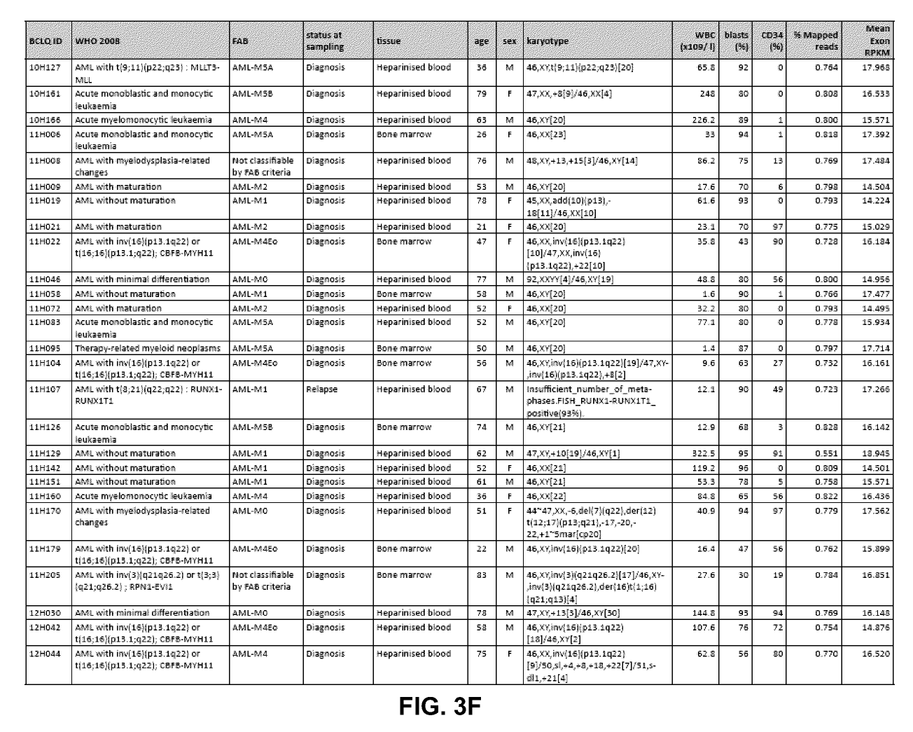

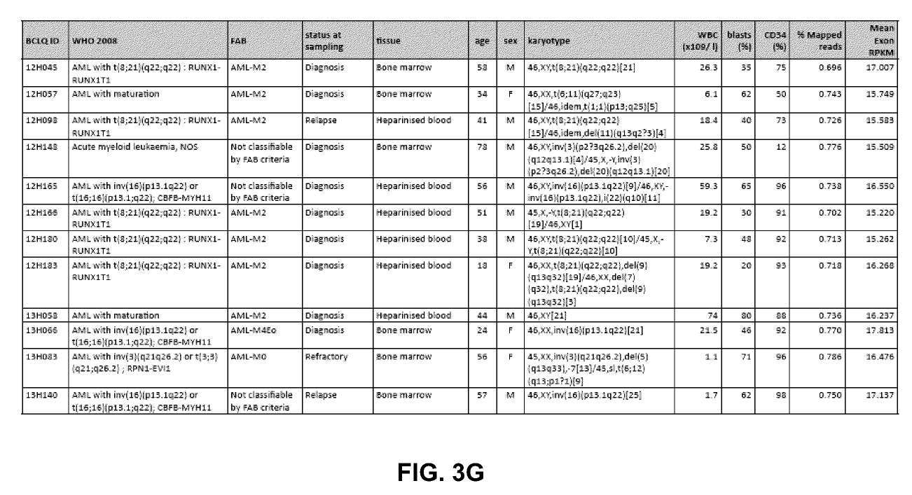

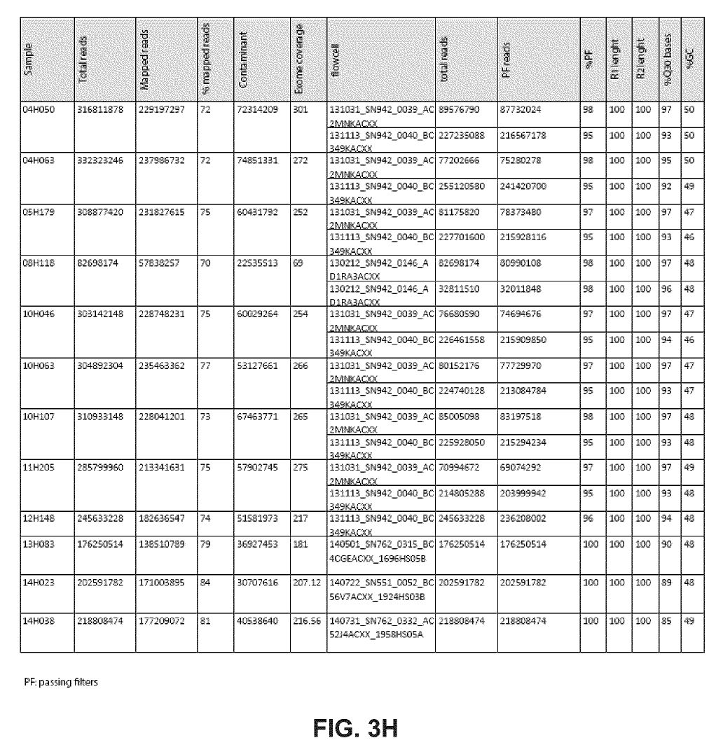

This study was approved by the Research Ethics Boards (REB) of Université de Montréal and Maisonneuve-Rosemont Hospital. Samples were collected between 2001 and 2015 according to Quebec Leukemia Cell Bank procedures (Fares et al., Science. 2014 Sep. 19; 345(6203):1509-12). Normal paired DNAs were obtained from buccal swabs or saliva. Ten EVI1-r AMLs were analyzed, including 6 with inv(3)/t(3;3) and 4 with other EVI1 rearrangements as well as a cohort of 143 AMLs from other cytogenetic groups (FIGS. 3A-G) and 17 control CB-derived normal CD34⁺ cells obtained from Héma-Québec and processed as described (Fares et al., Science. 2014 Sep. 19;345(6203):1509-12).

RNA and DNA Isolation.

RNA was isolated from primary AML cells using TRIzol® reagent according to the manufacturer's instructions (Invitrogen®/Life Technologies®) with an additional purification on RNeasy® mini columns (Qiagen®) to obtain high quality RNA. DNA was isolated and purified using DNeasy® protocols (Qiagen®). Integrity verification of isolated RNA was performed on a Bioanalyzer® 2100 with a RIN>8 deemed acceptable. For quantitative RT-PCR analyses, complementary DNA was generated from RNA using a Qiagen® QuantiTect® Reverse Transcription Kit (Qiagen®) according to manufacturer's protocols. For sequencing experiments, libraries were constructed with the TruSeq® RNA Sample Preparation Kit (Illumina®) according to manufacturer's protocols.

Sequencing and Bioinformatics Analysis.

Libraries were constructed with the TruSeq® RNA Sample Preparation Kit (Illumina). Sequencing was performed using an Illumina® HiSeq 2000 with 200 cycles paired end runs. Sequence data were mapped to the reference genome hg19 using the Illumina® Casava 1.8.2 package and Elandv2 mapping software according to RefSeq annotations (UCSC, Jan. 27, 2011 or Apr. 16, 2014). Mean coverage of EVI1-rearranged samples was 231X and individual reads were 100 bp long. Detailed sequencing and mapping statistics of the 12 EVI1-r AML is provided in FIG. 3H. Kaplan-Meier survival analysis associated PAWR expression to lower overall survival (N=168).

Mutation Identification and Validation.

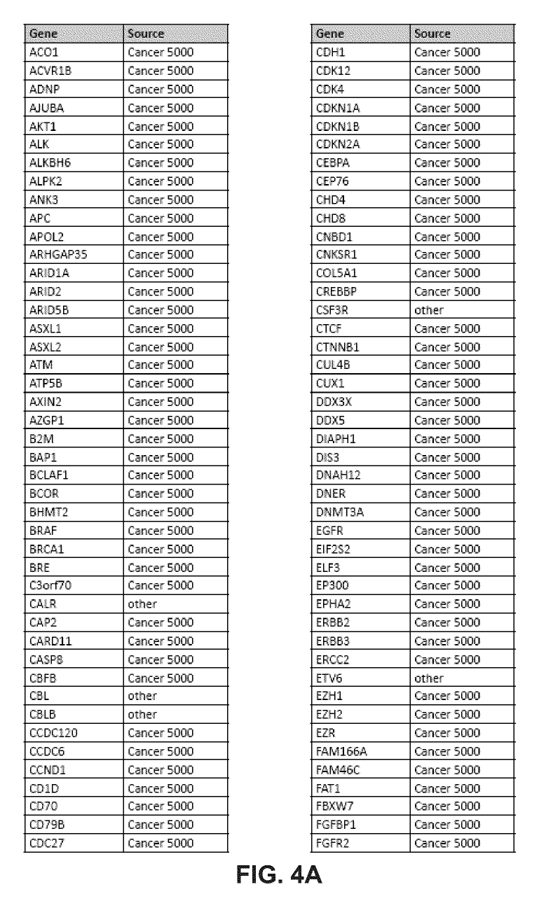

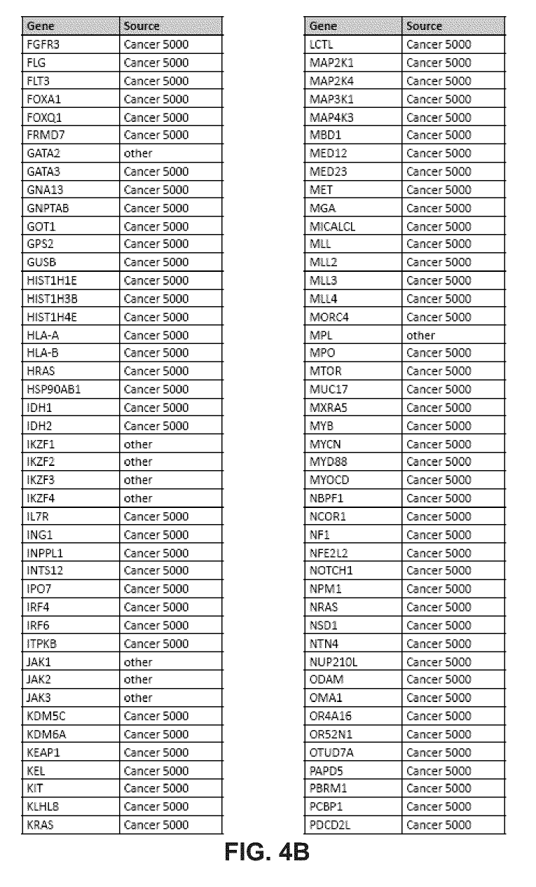

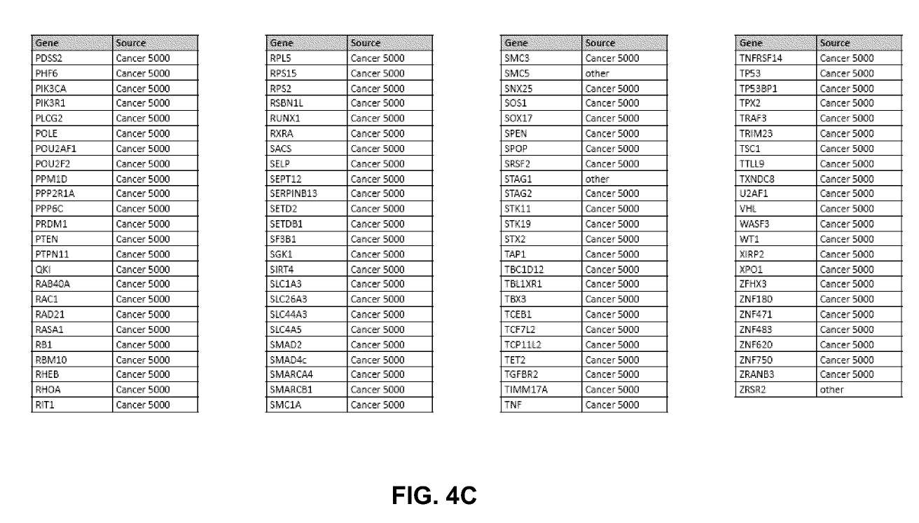

Variants were identified using Casava 1.8.2. Mutations outside of the coding region were excluded, and only nonsynonymous variants (SNP or Indel) were considered. Variants identified in normal controls (representing polymorphisms or sequencing artifacts) were filtered out. Known single nucleotide polymorphisms (SNP) (dbSNP, version 137) were also removed, except for those in known leukemia “hotspots”. All variants reported have a variant allelic frequency (VAF) >20%, ≧8 variant reads, ≧20 total reads and a quality score ≧20. Because NRAS and KRAS mutations are commonly found in minor clones (Kandoth C, et al. Nature. 2013; 502(7471):333-339), a VAF of ≧3% was tolerated if ≧15 variant reads were present in “hotspots” of those genes (N/KRAS G12, G13 and Q61). All variants in cancer-related genes (Lawrence M, et al. Nature. 2014; 505(7484):495-501) (FIGS. 4A-4C) are reported. Variants in non-cancer genes were only considered if recurrent in at least 2 EVI1-r AML samples. All recurrent mutations identified by next-generation sequencing (NGS) in the EVI1 cohort have been validated by Sanger sequencing of tumoral DNA or cDNA. The somatic or germline status of previously unreported variants (COSMIC database, version 68) were all confirmed by Sanger sequencing of DNA obtained at diagnosis from buccal swabs or saliva. Missense variants in ASXL1 were not considered, as they mostly represent germline polymorphisms (Schnittger S, et al. Leukemia. 2013; 27(1):82-91).

Expression.

Transcript levels are given as Reads Per Kilobase per Million mapped reads (RPKM) and genes are annotated according to RefSeq annotations (UCSC, Apr. 16, 2014). Splice isoforms were identified with Tophat 2.0.7 and Cufflinks 2.1.1., and are expressed in Fragments Per Kilobase of exon per Million fragments mapped (FPKM)).

Generation of a Full Leucegene-Specific Transcriptome Annotation.

Complementary to the Elandv2 mapping based on RefSeq annotations, a final “ab initio” transcriptome assembly was generated based on raw sequence data using Tophat/Cufflinks methodology. This pipeline, which had served to explore new putative non-coding genes, was refined to accommodate the simultaneous detection of new intergenic transcripts as well as novel splice isoforms of already annotated genes. This analysis utilized the Gencode (version 19) annotation, a significantly more comprehensive transcriptome annotation than Refseq, resulting in the discovery of 78,336 novel splice isoforms, 2,607 new intergenic transcribed loci (with 4,814 transcripts) and 1,789 new antisense transcripts. The annotation of novel genes/transcripts was merged with the Gencode (v19) annotation to obtain a complete Leucegene specific transcriptome catalog comprising roughly 51,000 genes and almost 280,000 transcripts. FPKM expression values on both isoform and gene levels across all Leucegene AML and control samples were computed using Cufflinks.

Quantitative PCR Analysis.

Quantitative PCR was performed using the TaqMan® Gene Expression system (Applied Biosystems®) on an Applied Biosystems® 7500 and in 0.2 ml 96-well polypropylene transparent PCR plates (Sarstedt®). Primer sequences were the following: ABL1-F 5′-TGGAGATAACACTCTAAGCATAACTAAAGGT-3′ (SEQ ID NO: 1), ABL1-R 5′-GATGTAGTTGCTTGGGACCCA-3′ (SEQ ID NO: 2), PAWR-F 5′-TGGTCAACATCCCTGCCG-3′ (SEQ ID NO: 3), PAWR-R 5′-TTGCATCTTCTCGTTTCCGC-3′ (SEQ ID NO: 4), FAM/ZEN/IBFQ probe sequences were the following: ABL1 5′-CCATTTTTGGTTTGGGCTTCACACCATT-3′ (SEQ ID NO: 5), PAWR 5′-AGTACGAAGATGATGAAGCAGGGC-3′ (SEQ ID NO: 6). Plasmid standard curves were developed for ABL1 and PAWR on pMA-T vectors. Data analysis was performed on the Applied Biosystems® 7500 software v2.0.5, with the threshold set at 0.1 and baseline set between cycles 3 and 15.

Statistics.

Fisher's exact test was used in the analysis of contingency tables. Analysis of differential gene expression was performed with the Wilcoxon rank-sum test (Mann-Whitney) using the stats R package (http://cran.r-project.org/) with estimation of the False-discovery rate (FDR, q-value). In order to avoid issues with log-scale representation of RPKM equal to zero or normalized copy numbers equal to zero, a small constant (0.0001 or 0.01, respectively) was added to all expression values when log transformation was performed. For scatterplot visualizations, averages of groups were performed on log 10 transformed values to avoid overrepresentation of extremes. Survival analyses were performed in R using the CRAN package ‘survival’. A log-rank test was applied to compare the survival curves and determine if they were equivalent. The resulting p-value was reported on the corresponding Kaplan-Meier plots.

### Example 2: Analysis of Acquired Mutations in EVI1 AML

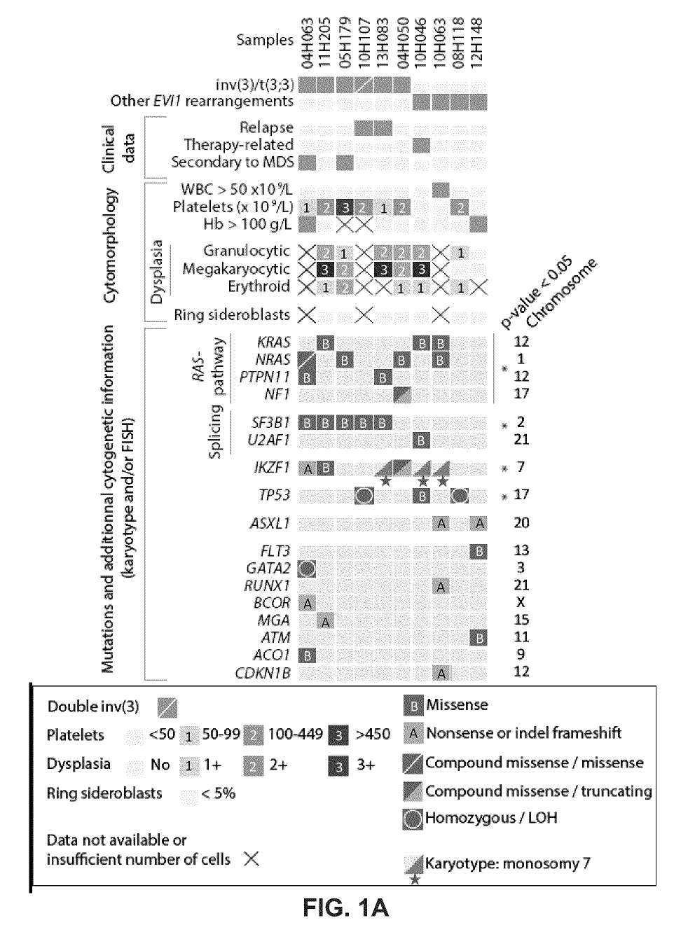

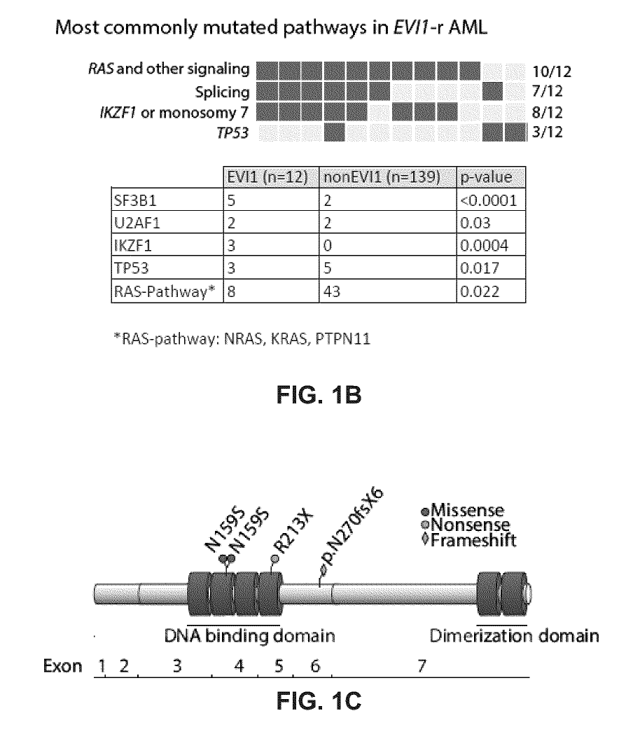

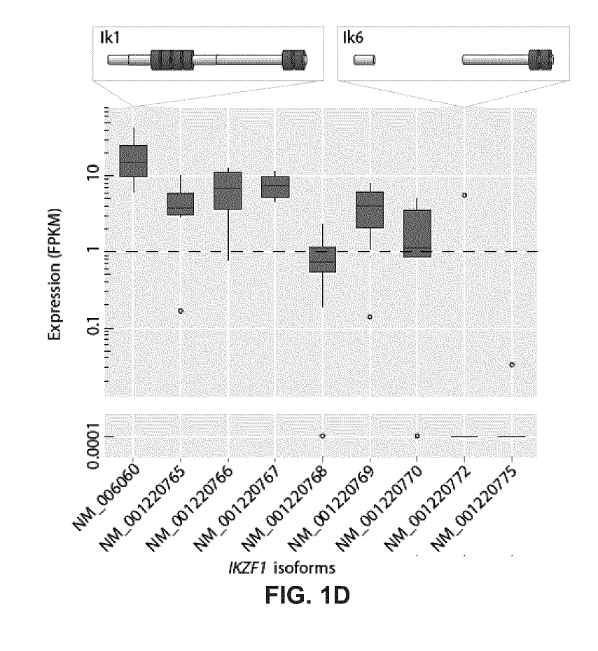

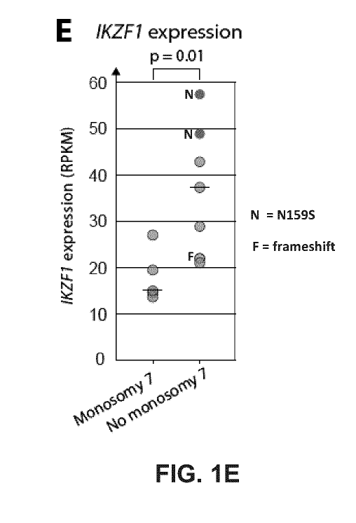

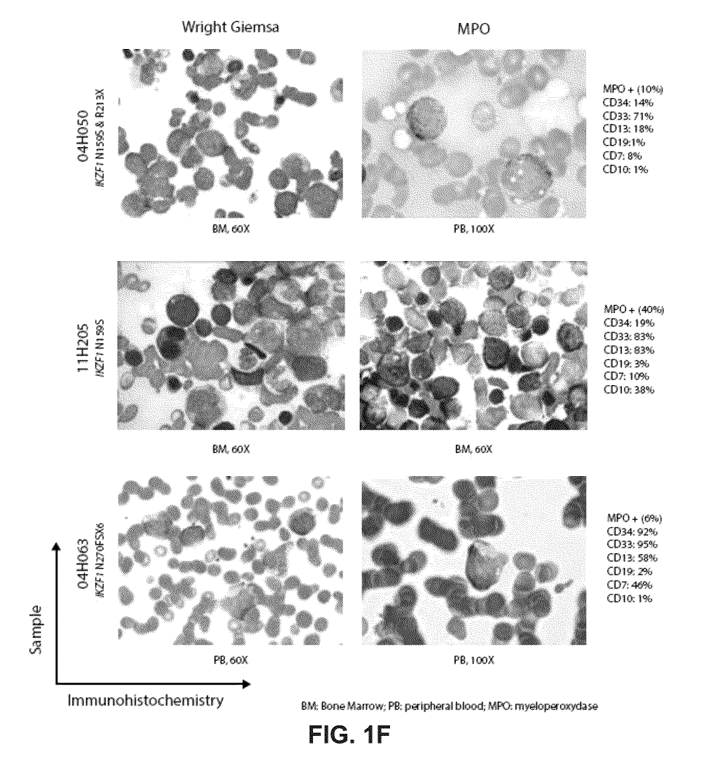

Twelve EVI1-r AMLs were analyzed, including 7 with inv(3)/t(3;3) and 5 with other EVI1 rearrangements as well as a cohort of 139 AMLs from other cytogenetic groups and 17 control CB-derived normal CD34⁺ cells (clinical and laboratory characteristics and sequencing statistics are detailed in FIGS. 3A to 3G). The most frequent mutations in EVI1-r AMLs are presented in FIGS. 1A and 1B and 5A to 5C.

RAS Mutations

RAS-pathway mutations (NRAS, KRAS, PTPN11, NF1) were significantly more frequent (7/10, 70%) in EVI1-r AMLs than in other AMLs sequenced as part of this project (43/139, p=0.022). Allelic frequency determination revealed that at least one gene of this pathway was mutated in the dominant clone contributing to a cumulative ratio of mutated/wild type alleles of ˜50% (FIGS. 5A to 5C) possibly indicating a strong collaboration between RAS-pathway activation and EVI1. Two additional samples had mutations in KIT and FLT3, hence an activated signaling mutation was present in 10/12 samples (FIG. 1B).

SF3B1 and Other Splicing Factors

Splicing was the second most commonly mutated pathway (7/12). SF3B1 mutations were found in 5/10 samples (50%), all within the inv(3)/t(3;3) subgroup (5/6, 83%), and at a lower frequency than in the control cohort (FIG. 1B, p<0.0001). Mutations were all located in exons 14-15 corresponding to the same “hotspots” as found in myelodysplastic syndromes (MDS): K700E (n=3), G740E (n=1) and R625C (n=1). In MDS, SF3B1 mutations are associated to ring sideroblasts (RS) and normal or elevated platelet counts^{13,14}. In the cohort studied herein, RS were universally absent and platelet counts were not different between SF3B1 mutated and wild-type samples (median 109 vs 88×10⁹/L respectively, p=0.34; FIG. 1B). Another splicing factor, U2AF1, was found mutated in 2 additional samples (FIG. 1A) also in same positions than in MDS (Q157) and at a higher frequency than in control group (p=0.03).

IKZF1

Four IKZF1 mutations were found in 3 EVI1-r AMLs (30%) compared to none in the control cohort (FIGS. 1B-C, p<0.0001). Although mutations of this gene were previously reported in BCR-ABL1 acute lymphoblastic leukemia (ALL)¹⁵ and in advanced myeloproliferative neoplasms¹⁶, they were never found at high frequency in any AML cohort. IKZF1 mutated samples displayed typical AML characteristics (FIG. 1F). In ALL, the frequent deletion of IKZF1 exons 3-6 (Δ3-6) compromises the DNA-binding activity of this protein. Such deletions were not found in the cohort studied herein. Rather, non-synonymous or truncating mutations are observed (e.g., N159S (n=2), R213X, N270fsx6: FIG. 1D). Interestingly, the most frequent mutation (2/10 samples), N159S, occurs in a position involved in DNA-binding¹⁶.

The reported association between monosomy 7 and EVI1-r AMLs is most interesting considering that IKZF1 is located on this chromosome and that IKZF1 expression levels were lower in all 3 samples with monosomy 7 (FIG. 1E). Interestingly, monosomy 7 and IKZF1 mutations were never observed simultaneously (see dark triangles with a star underneath representing monosomy 7 specimens in FIG. 1A) possibly indicating that IKZF1 is more important than anticipated in this disease. This is reminiscent of what has been found in ALL where IKZF1 alterations are either limited to the gene itself (intra-genic deletions or mutations) or involve larger chromosomal deletions, predicted to result in the expression of a dominant-negative isoform or in haploinsufficiency respectively^{15,18}.

Other Mutations

Mutations were recurrently found in 2 additional genes, namely TP53 (3/10, 30%) and ASXL1 (2/10, 20%: FIGS. 1A-B). Additional non-recurrent mutations are illustrated in FIG. 1A. Among those, 1 sample had a GATA2 mutation with mono-allelic expression (VAF 91%, FIG. 5A). This is most interesting considering the recent data showing that MECOM overexpression in 3q rearrangements AML is driven by an enhancer of the GATA2 gene¹⁹.

### Example 3: Transcriptome Analysis

MECOM

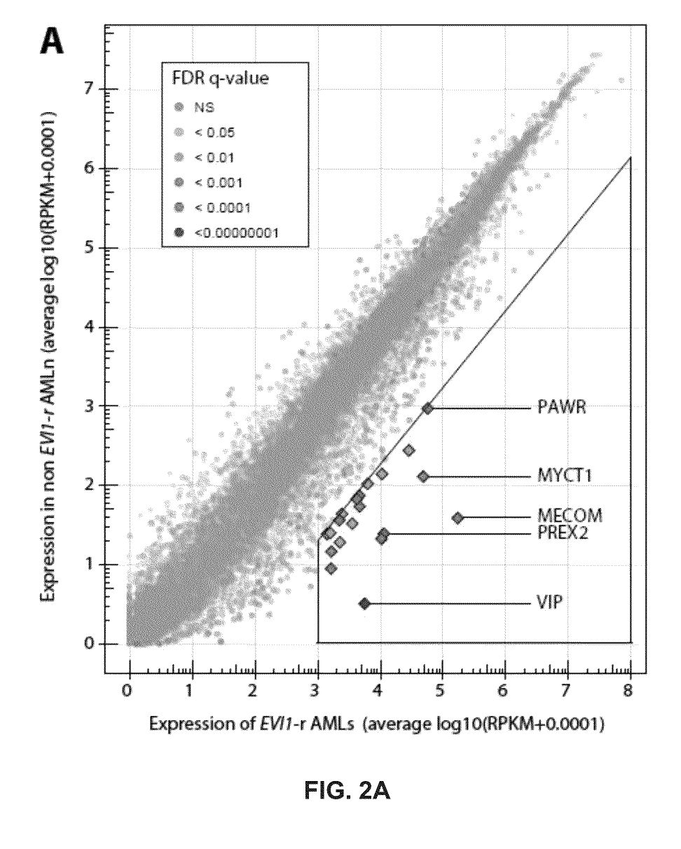

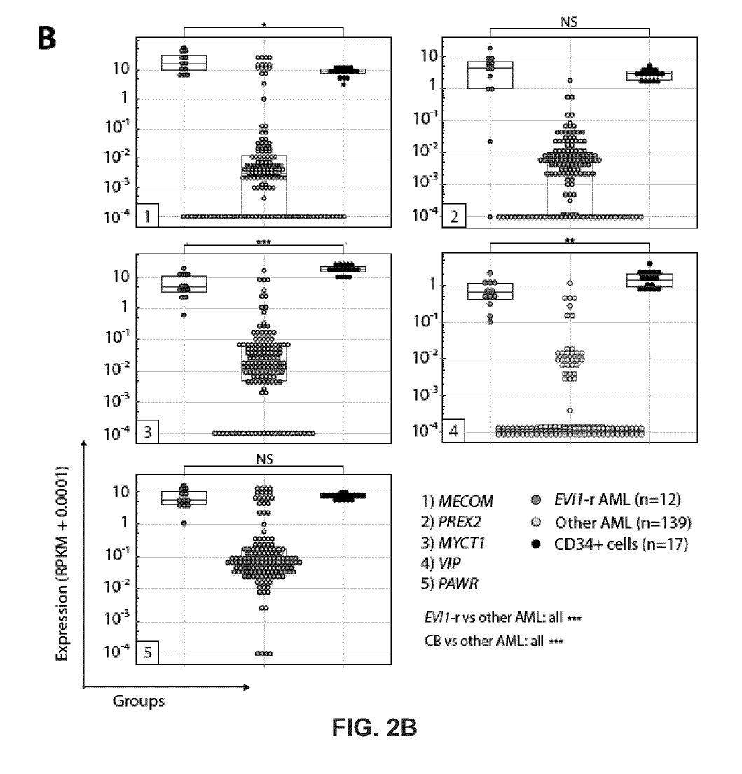

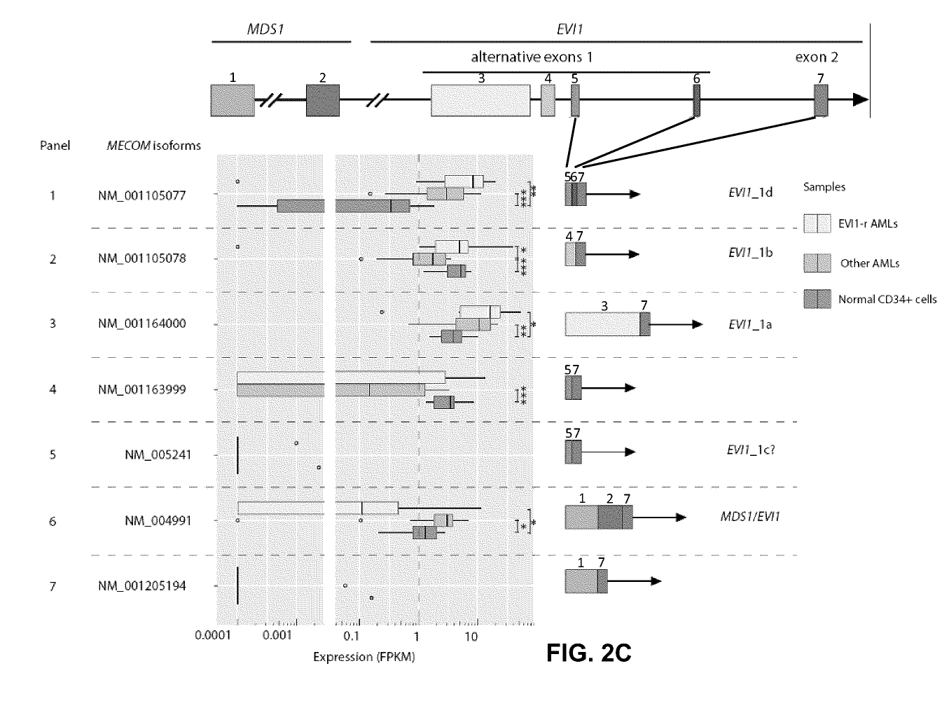

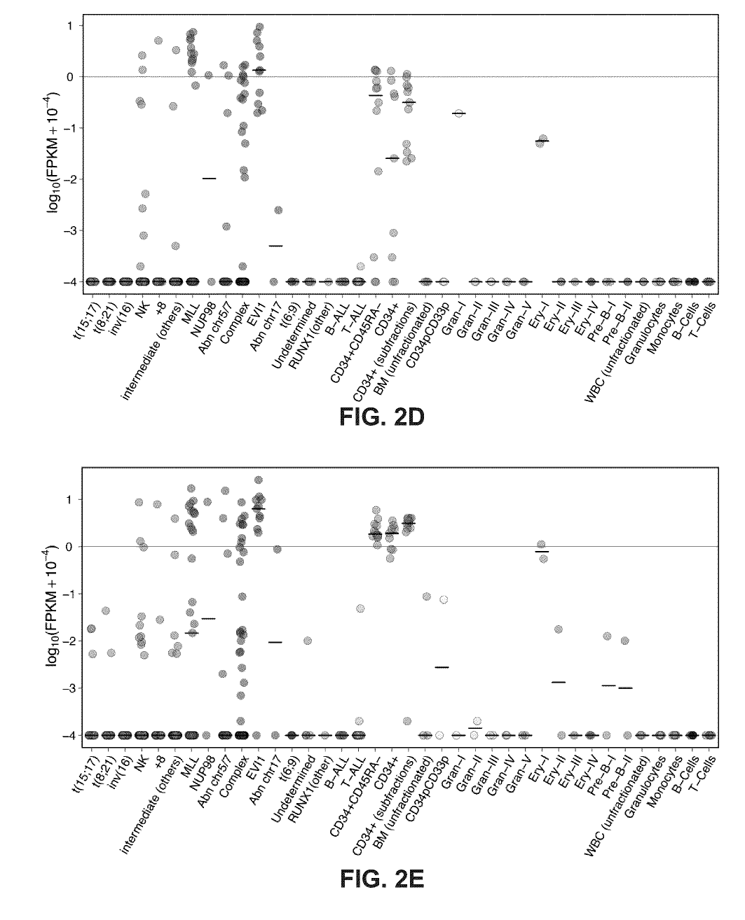

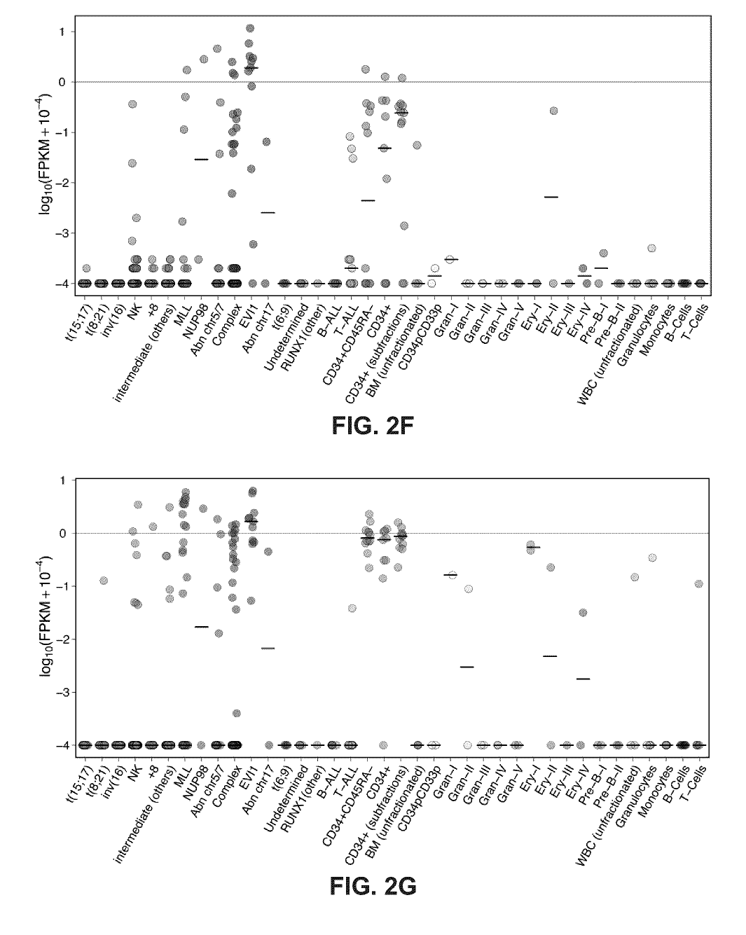

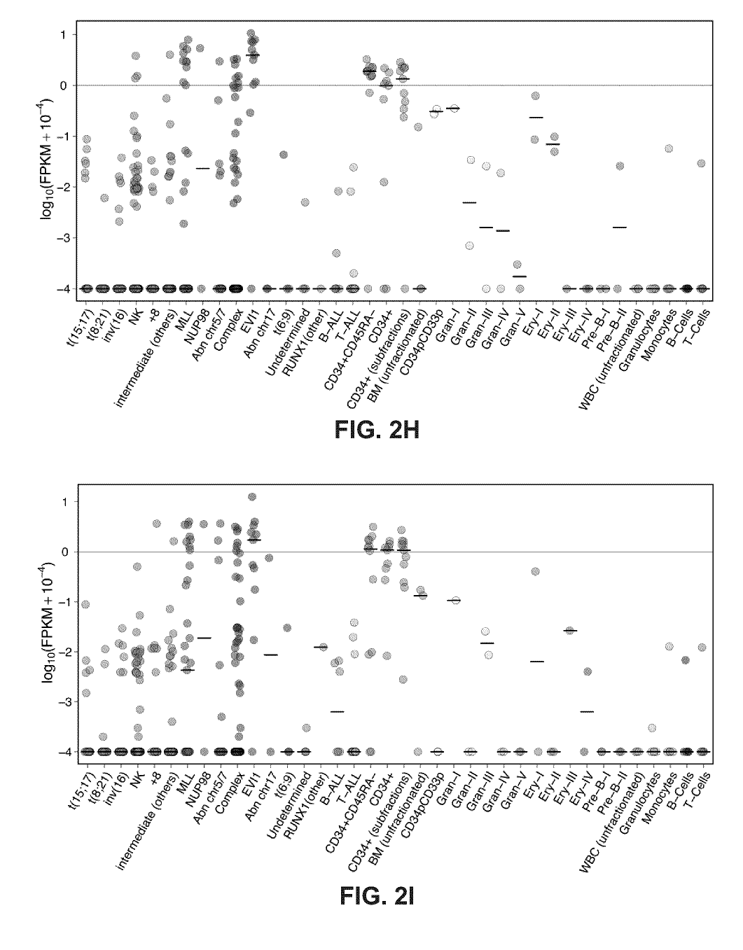

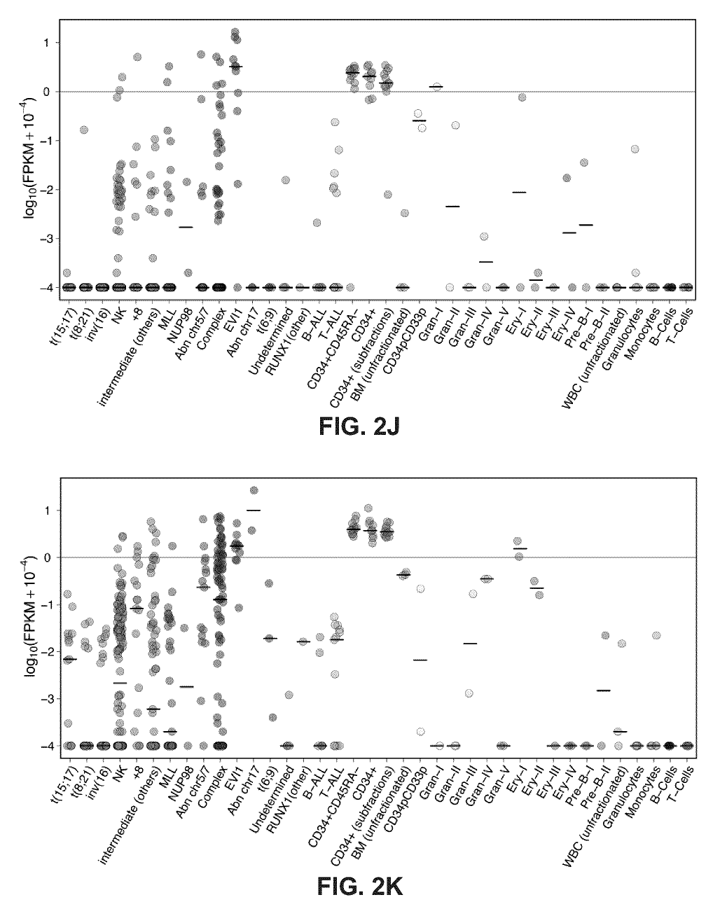

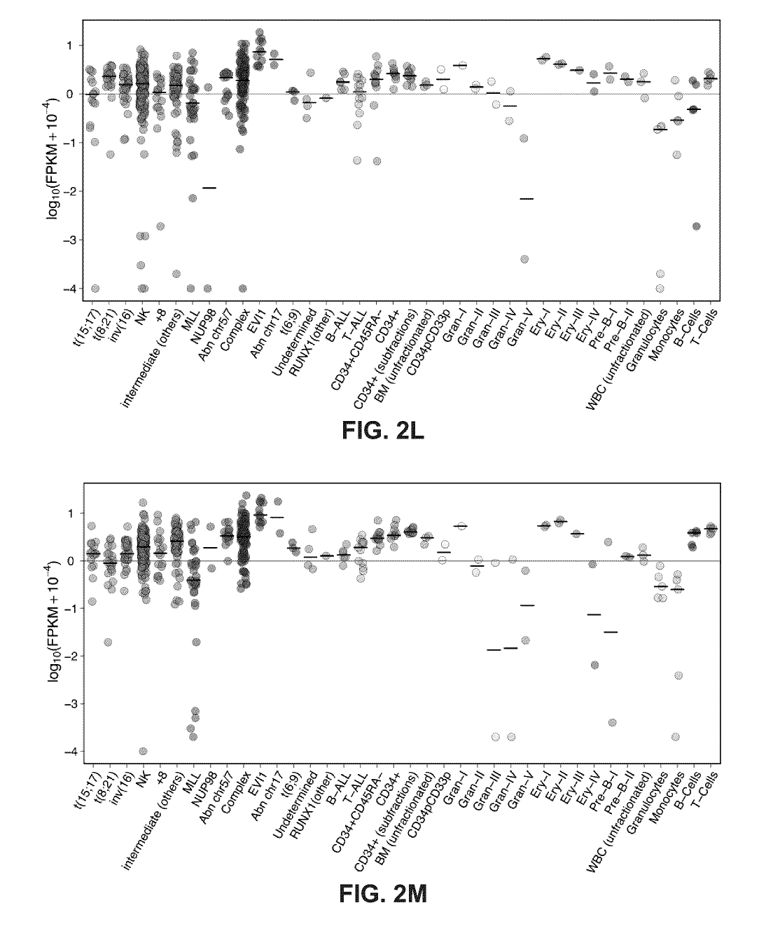

Many genes were differentially expressed in AML with EVI1-r AMLs compared to other non EVI1-r leukemias, and the 20 most preferentially expressed genes are detailed in FIG. 6A. The gene with the greatest relative overexpression was MECOM (FIG. 2A). MECOM was expressed in all EVI1-r samples, in 11.5% of non-EVI1-r leukemias (16/139) and in all normal CD34⁺ cells (FIG. 2B). As previously reported³, EVI1-r AMLs showed reduced expression of the long MDS1/EVI1 isoform when compared to other leukemias or normal CD34⁺ cells (compare white bars (EVI1-r AMLs) to other AMLs (light gray) and normal CD34⁺ cells (darker gray) in panel 6 in FIG. 2C). EVI1 and non-EVI1 rearranged AML samples both expressed significantly higher levels of short isoforms 1d (panel 1) and 1a (panel 3) when compared to normal CD34⁺ cells (FIG. 2C), which preferentially expressed transcript NM_001163999 (panel 4). The novel MECOM isoform transcripts depicted in Table 4 were also shown to be preferentially expressed in EVI1-r AMLs:

TABLE 4
Novel MECOM, MYCT1 and LRBA isoform transcripts
preferentially expressed in EVI1-r AMLs

| transcript_name | locus | class_code | factor | q.value |
|---|---|---|---|---|
| MECOM_iso_16 | chr3: 168801214-169381661 | j | 1138.9 | 9.97E−16 |
| MECOM_iso_22 | chr3: 168801214-169381661 | = | 111.0825137 | 7.09E−10 |
| MECOM_iso_20 | chr3: 168801214-169381661 | = | 12.1 | 1.05E−10 |
| MECOM_iso_29 | chr3: 168801214-169381661 | = | 10.53994674 | 3.60E−14 |
| MECOM_iso_28 | chr3: 168801214-169381661 | = | 10.08766593 | 2.13E−07 |
| MECOM_iso_5 | chr3: 168801214-169381661 | j | 1.784632035 | 2.17E−06 |
| MECOM_iso_21 | chr3: 168801214-169381661 | = | 1.686888454 | 3.42E−08 |
| MYCT1_iso_6 | chr6: 153018904-153081019 | = | 1.057197739 | 0.000135345 |
| LRBA_iso_6 | chr4: 151185593-151936879 | = | 1.048743151 | 3.55E−05 |
| LRBA_iso_3 | chr4: 151185593-151936879 | = | 1.012550168 | 5.96E−05 |

class code = transcript identifier (j: not previously annotated, =: previously annotated)
factor = ratio between median FPKM expression in EVI1-r AML vs. median FPKM in control groups
q.value = Mann-Whitney test with false discovery rate (FDR)

Other Preferentially Expressed Genes

Of the other overexpressed genes, PREX2, VIP, MYCT1, and PAWR, highlighted in FIG. 2A, displayed the greatest specificity for EVI1-r AML. Interestingly, those genes were also all expressed in normal CD34⁺ cells (FIG. 2B). A novel isoform of MYCT1, as well as two isoforms of LRBA, were also overexpressed in EVI1-r AML (Table 4).

PREX2 gene is a PIPS-dependent Rac exchanger²⁰ and it amplifies PI3K signaling²¹. This dual activity could lead to RAC and AKT (PI3K) pathway activation. PREX2 is recurrently mutated in malignant melanoma²². PAWR (PRKC, Apoptosis, WT1, Regulator) encodes for a proapoptotic protein inactivated by PI3K/AKT signaling and participating in PTEN mediated apoptosis²³. MYCT1 (MYC target 1) is a direct target of c-MYC. VIP (vasointestinal peptide) is located in same locus than MYCT1 on chromosomal band 6q25.2. It has been described as a potential growth promoting factor in hematopoietic stem and progenitor cells²⁶ and it may have a role in megakaryocytic proliferation²⁷. LRBA (LPS-responsive vesicle trafficking, beach and anchor containing) is induced in B cells and macrophages by bacterial lipopolysaccharides (LPS), may be involved in leading intracellular vesicles to activated receptor complexes.

Based on the results presented herein, EVI1-r AMLs may be defined as a group of leukemias characterized by anomalies in several genetic networks including RAS/signaling, splicing (SF3B1), and possibly IKZF1, and a distinct expression signature.

### Example 4: PAWR is a Suitable Prognostic Marker of AML, Including Intermediate-Risk AML

It was next tested whether PAWR, which was shown to be one of the most differentially expressed genes in EVI1-r AMLs (a subgroup associated with very poor overall survival), could be used as a prognostic marker for AMLs, i.e. to predict treatment outcome and/or patient survival.

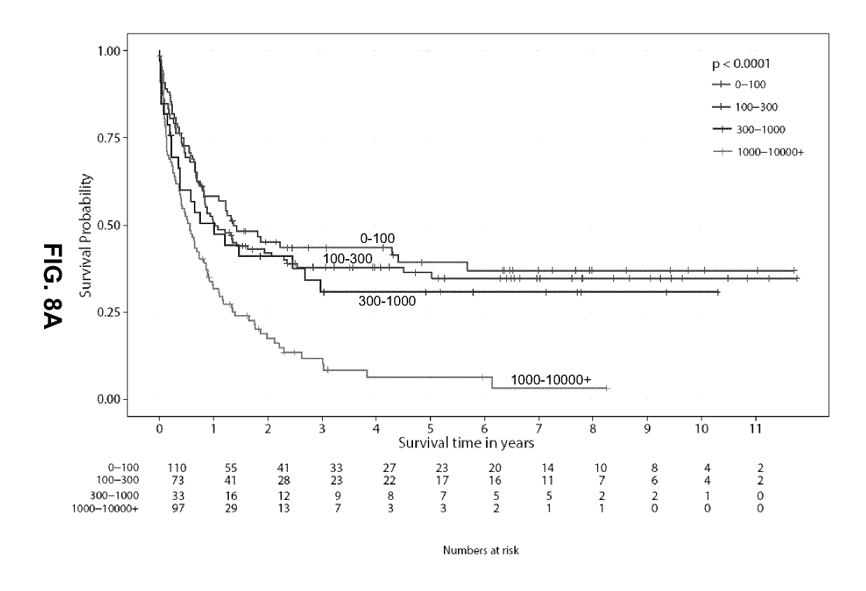

It was first determined whether the transcriptome data could be reproduced in a quantitative RT-PCR assay. FIG. 7 shows the very good correlation (r=0.9049) in PAWR expression levels detected using either RNA-Seq transcriptome data (log RPKM) or the real-time quantitative RT-PCR assay described herein (normalized copy number, NCN). The prognostic value of PAWR was thus evaluated using a real-time quantitative RT-PCR assay.

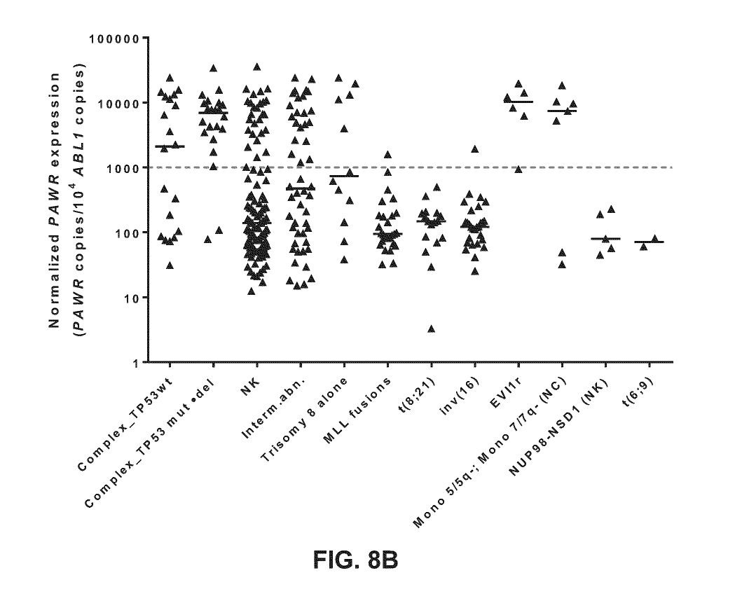

The results depicted in FIGS. 8A and 8B show the large dynamic range in PAWR expression levels between genetic subtypes in the Leucegene AML cohort. A significant difference in overall survival was observed in samples expressing greater than or equal to 1000 normalized copy numbers, thereby identifying a potential cut-off value (FIG. 8A). FIG. 8B shows the expression levels of genetic subtypes within the Leucegene AML cohort as determined using the PAWR quantitative RT-PCR assay, which demonstrate that greater than 1000 normalized copy number expression of PAWR was typically associated with genetic subtypes with known adverse clinical outcome. However, several additional specimens that were either normal karyotype, intermediate abnormal AML, or otherwise not associated with known genetic subgroups of poor clinical outcome (typically referred to as “intermediate-risk” AMLs) were shown to have PAWR expression values above the set cut-off value, suggesting that PAWR expression could potentially be used to predict poor clinical outcome in AML patients, including in intermediate-risk AML patients.

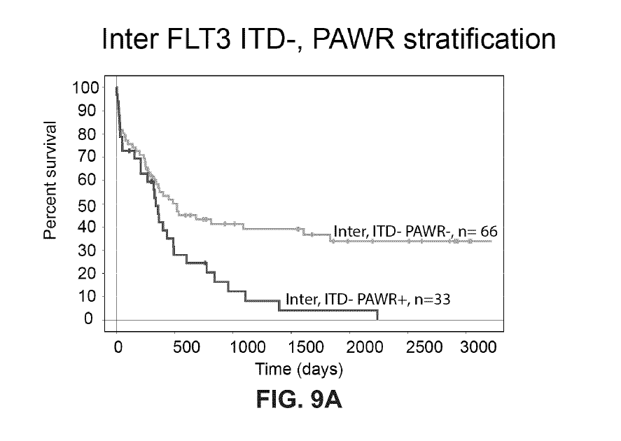

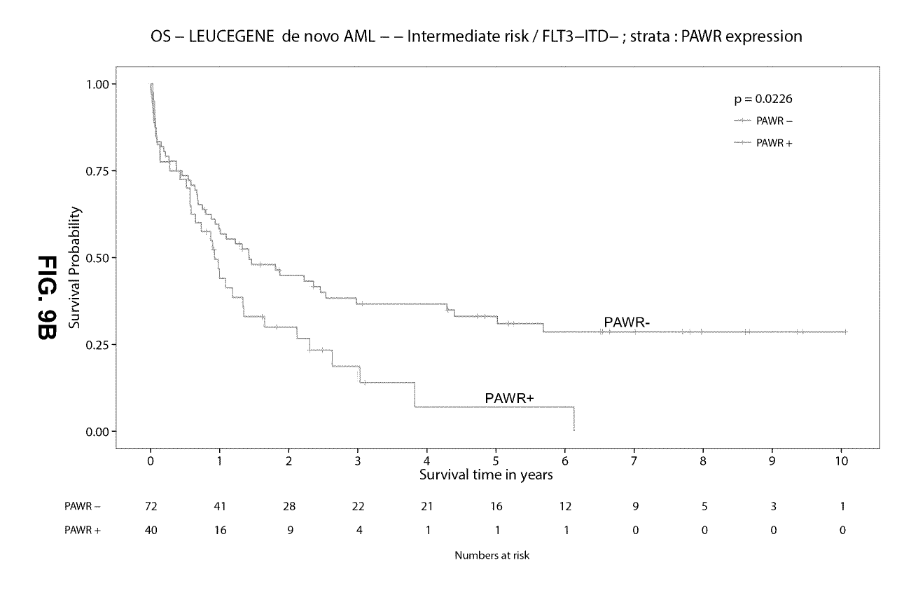

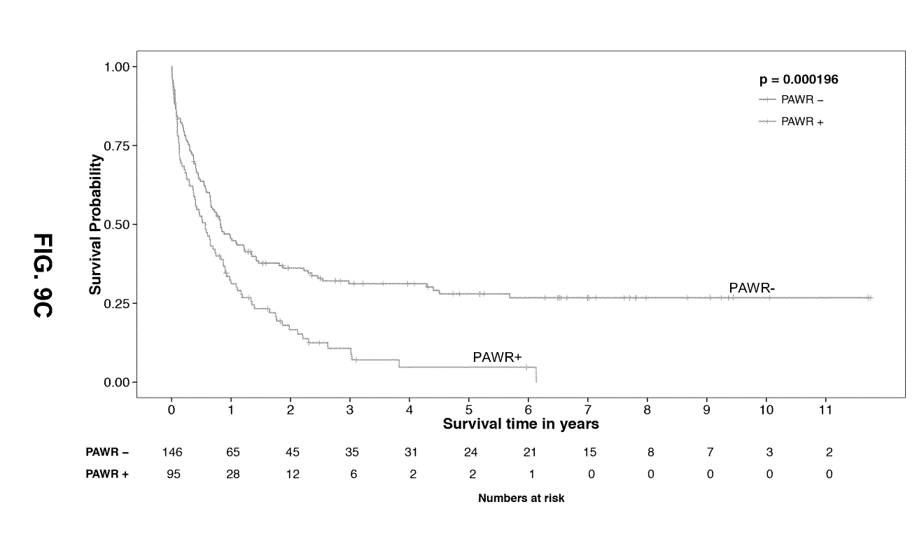

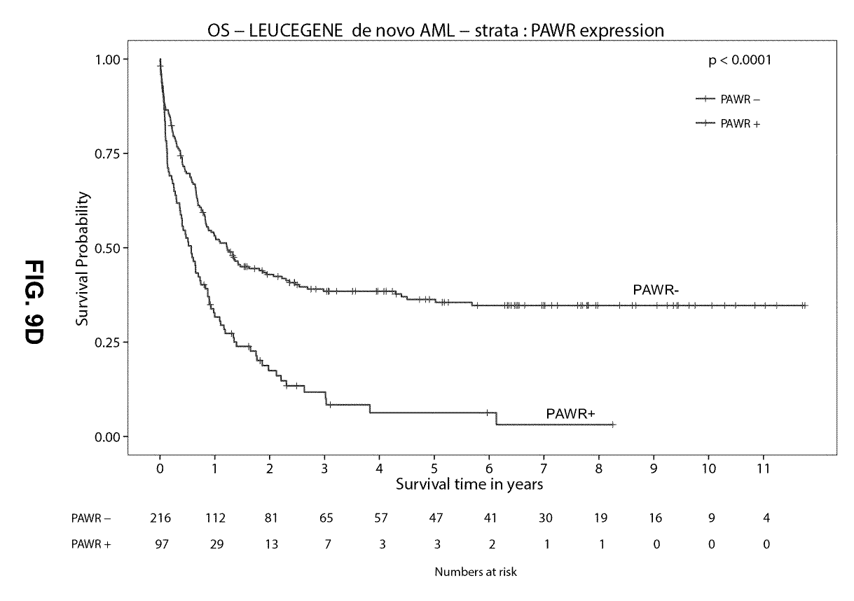

The presence of FLT3 aberrations, notably internal tandem duplication (ITD), is typically associated with an unfavorable clinical outcome. However, the prediction of the clinical outcome (i.e. good vs. poor prognosis) of FLT3-ITD negative AML patients is much more difficult. Thus, the possibility of re-stratifying these “intermediate-risk” patients into good and poor outcome based on PAWR expression was assessed. FIGS. 9A and 9B show the overall survival curves according to PAWR expression in Leucegene de novo AML Intermediate Risk FLT3-ITD-negative cohort determined by RNA-Seq (FIG. 9A) and quantitative RT-PCR (FIG. 9B), which demonstrate poor overall survival in specimens whose PAWR expression is >1 RPKM (FIG. 9A) or ≧1000 normalized PAWR copy number per 10⁴ ABL1 copy number (FIG. 9B). These results confirm that PAWR expression may be used as a marker to predict the clinical outcome in intermediate-risk FLT3-ITD-negative patients.

It was next tested whether PAWR expression may be used as a marker to predict the clinical outcome in the general AML patient population, i.e. irrespective of the AML subgroup. FIG. 9D depicts the overall survival curves according to PAWR expression determined by quantitative RT-PCR in the Leucegene AML cohort, which demonstrates the poor overall survival by specimens whose PAWR expression is ≧1000 normalized copy number (NCN) relative to specimens <1000 NCN. Similar results were obtained when AML patients with a favorable prognosis (t(8;21) and inv(16)) and for which the PAWR test is less informative (AML with MLL fusions) were excluded from the survival analysis (FIG. 9C). These results confirm that PAWR expression levels may be used as a marker to predict the clinical outcome in AML patients.

Although the present invention has been described hereinabove by way of specific embodiments thereof, it can be modified, without departing from the spirit and nature of the subject invention as defined in the appended claims. The scope of the claims should not be limited by the preferred embodiments set forth in the examples, but should be given the broadest interpretation consistent with the description as a whole. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Steinleitner K, Rampetsreiter P, Köffel R, et al. EVI1 and MDS1/EVI1 expression during primary human hematopoietic progenitor cell differentiation into various myeloid lineages. Anticancer research. 2012; 32(11):4883-4889.
2. Barjesteh van Waalwijk van Doorn-Khosrovani S, Erpelinck C, van Putten W, et al. High EVI1 expression predicts poor survival in acute myeloid leukemia: a study of 319 de novo AML patients. Blood. 2003; 101(3):837-845.
3. Lugthart S, van Drunen E, van Norden Y, et al. High EVI1 levels predict adverse outcome in acute myeloid leukemia: prevalence of EVI1 overexpression and chromosome 3q26 abnormalities underestimated. Blood. 2008; 111(8):4329-4337.
4. Gröschel S, Schlenk R, Engelmann J, et al. Deregulated expression of EVI1 defines a poor prognostic subset of MLL-rearranged acute myeloid leukemias: a study of the German-Austrian Acute Myeloid Leukemia Study Group and the Dutch-Belgian-Swiss HOVON/SAKK Cooperative Group. Journal of clinical oncology: official journal of the American Society of Clinical Oncology. 2013; 31(1):95-103.
5. Grimwade D, Hills R K, Moorman A V, et al. Refinement of cytogenetic classification in acute myeloid leukemia: determination of prognostic significance of rare recurring chromosomal abnormalities among 5876 younger adult patients treated in the United Kingdom Medical Research Council trials. Blood. 2010; 116(3):354-365.
6. Rogers H, Vardiman J, Anastasi J, et al. Complex or monosomal karyotype and not blast percentage is associated with poor survival in acute myeloid leukemia and myelodysplastic syndrome patients with inv(3)(q21q26.2)/t(3;3)(q21;q26.2): a Bone Marrow Pathology Group study. Haematologica. 2014; 99(5): 821-829.
7. Lugthart S, Groschel S, Beverloo H, et al. Clinical, molecular, and prognostic significance of WHO type inv(3)(q21q26.2)/t(3;3)(q21;q26.2) and various other 3q abnormalities in acute myeloid leukemia. Journal of clinical oncology: official journal of the American Society of Clinical Oncology. 2010; 28(24):3890-3898.
8. Weisser M, Haferlach C, Haferlach T, Schnittger S. Advanced age and high initial WBC influence the outcome of inv(3) (q21q26)/t(3;3) (q21;q26) positive AML. Leukemia & lymphoma. 2007; 48(11):2145-2151.
9. Rogers H J, Vardiman J W, Anastasi J, et al. Complex or monosomal karyotype and not blast percentage is associated with poor survival in acute myeloid leukemia and myelodysplastic syndrome patients with inv(3)(q21q26.2)/t(3;3)(q21;q26.2): a Bone Marrow Pathology Group study. Haematologica. 2014; 99(5):821-829.
10. Lugthart S, Groschel S, Beverloo H B, et al. Clinical, molecular, and prognostic significance of WHO type inv(3)(q21q26.2)/t(3;3)(q21;q26.2) and various other 3q abnormalities in acute myeloid leukemia. Journal of clinical oncology: official journal of the American Society of Clinical Oncology. 2010; 28(24):3890-3898.
11. Haferlach C, Bacher U, Haferlach T, et al. The inv(3)(q21q26)/t(3;3)(q21;q26) is frequently accompanied by alterations of the RUNX1, KRAS and NRAS and NF1 genes and mediates adverse prognosis both in MDS and in AML: a study in 39 cases of MDS or AML. Leukemia. 2011; 25(5):874-877.
12. Sun J, Konoplev S, Wang X, et al. De novo acute myeloid leukemia with inv(3)(q21q26.2) or t(3;3)(q21;q26.2): a clinicopathologic and cytogenetic study of an entity recently added to the WHO classification. Modern pathology: an official journal of the United States and Canadian Academy of Pathology, Inc. 2011; 24(3):384-389.
13. Papaemmanuil E, Cazzola M, Boultwood J, et al. Somatic SF3B1 mutation in myelodysplasia with ring sideroblasts. The New England Journal of Medicine. 2011; 365(15):1384-1395.
14. Jeromin S, Haferlach T, Grossmann V, et al. High frequencies of SF3B1 and JAK2 mutations in refractory anemia with ring sideroblasts associated with marked thrombocytosis strengthen the assignment to the category of myelodysplastic/myeloproliferative neoplasms. Haematologica. 2013;98(2):7.
15. Mullighan C G, Miller C B, Radtke I, et al. BCR-ABL1 lymphoblastic leukaemia is characterized by the deletion of Ikaros. Nature. 2008; 453(7191):110-114.
16. Jager R, Gisslinger H, Passamonti F, et al. Deletions of the transcription factor Ikaros in myeloproliferative neoplasms. Leukemia. 2010; 24(7):1290-1298.
17. Cobb B S, Morales-Alcelay S, Kleiger G, Brown K E, Fisher A G, Smale S T. Targeting of Ikaros to pericentromeric heterochromatin by direct DNA binding. Genes & Development. 2000; 14(17):21462160.
18. Mullighan C, Su X, Zhang J, et al. Deletion of IKZF1 and Prognosis in Acute Lymphoblastic Leukemia. The New England Journal of Medicine. 2009; 360(5):470480.
19. Groschel S, Sanders M A, Hoogenboezem R, et al. A single oncogenic enhancer rearrangement causes concomitant EVI1 and GATA2 deregulation in leukemia. Cell. 2014; 157(2):369-381
20. Pandiella A, Montero J. Molecular pathways: P-Rex in cancer. Clinical cancer research: an official journal of the American Association for Cancer Research. 2013; 19(17):4564-4569.
21. Fine B, Hodakoski C, Koujak S, et al. Activation of the PI3K pathway in cancer through inhibition of PTEN by exchange factor P-REX2a. Science (New York, N.Y.). 2009; 325(5945):1261-1265.
22. Berger M, Hodis E, Heffernan T, et al. Melanoma genome sequencing reveals frequent PREX2 mutations. Nature. 2012; 485(7399):502-506.
23. Goswami A, Ranganathan P, Rangnekar V M. The phosphoinositide 3-kinase/Akt1/Par-4 axis: a cancer-selective therapeutic target. Cancer research. 2006; 66(6):2889-2892.
24. Yin X, Grove L, Rogulski K, V. Prochownik E. Myc Target in Myeloid Cells-1, a Novel c-Myc Target, Recapitulates Multiple c-Myc Phenotypes. Journal of Biological Chemistry. 2002; 277(22):19998-20010.
25. Kustikova O S, Schwarzer A, Stahlhut M, et al. Activation of Evil inhibits cell cycle progression and differentiation of hematopoietic progenitor cells. Leukemia. 2013; 27(5):1127-1138.
26. Kawakami M, Kimura T, Kishimoto Y, et al. Preferential expression of the vasoactive intestinal peptide (VIP) receptor VPAC1 in human cord blood-derived CD34&plus;CD38− cells: possible role of VIP as a growth-promoting factor for hematopoietic stem&sol;progenitor cells. Leukemia. 2004; 18(5):912-921.
27. Nam C, Case A J, Hostager B S, O'Dorisio M S. The role of vasoactive intestinal peptide (VIP) in megakaryocyte proliferation. Journal of molecular neuroscience: MN. 2009; 37(2):160-167.

1. A method for the disease prognosis of a subject suffering from acute myeloid leukemia (AML), said method comprising: measuring the level of expression of PRKC Apoptosis WT1 Regulator (PAWR) in a biological sample comprising leukemic cells from said subject; and comparing said level of expression to a threshold reference level, wherein a level of expression that is above said threshold reference level is indicative of a poor disease prognosis, wherein said method comprises amplifying a nucleic acid encoding PAWR using a first PAWR primer and a second PAWR primer.

2-3. (canceled)

4. The method of claim 1, wherein said first PAWR primer comprises at least 10 nucleotides of the sequence 5′-TGGTCAACATCCCTGCCG-3′ (SEQ ID NO:3).

5. The method of claim 4, wherein said first PAWR primer comprises the sequence 5′-TGGTCAACATCCCTGCCG-3′ (SEQ ID NO:3).

6. The method of claim 5, wherein said second PAWR primer comprises at least 10 nucleotides of the sequence 5′-TTGCATCTTCTCGTTTCCGC-3′ (SEQ ID NO:4).

7. The method of claim 6, wherein said second PAWR primer comprises the sequence 5′-TTGCATCTTCTCGTTTCCGC-3′ (SEQ ID NO:4).

8. The method of claim 1, wherein said method comprises detecting the nucleic acid encoding PAWR using a PAWR probe.

9. The method of claim 8, wherein said PAWR probe comprises (i) at least about 10 nucleotides of the sequence 5′-AGTACGAAGATGATGAAGCAGGGC-3′ (SEQ ID NO:6); or (ii) the sequence 5′-AGTACGAAGATGATGAAGCAGGGC-3′ (SEQ ID NO:6).

10. (canceled)

11. The method of claim 1, wherein the level of expression of PAWR is measured by quantitative reverse transcription polymerase chain reaction (RT-qPCR).

12. The method of claim 1, wherein said method further comprises normalizing the level of expression of PAWR based on the level of expression of a housekeeping gene.

13. The method of claim 12, wherein said housekeeping gene is ABL1 and said method comprises amplifying a nucleic acid encoding ABL1 using a first ABL1 primer and a second ABL1 primer.

14. (canceled)

15. The method of claim 13, wherein (a) said first ABL1 primer comprises (i) at least 10 nucleotides of the sequence 5′-TGGAGATAACACTCTAAGCATAACTAAAGGT-3′ (SEQ ID NO:1); or (ii) the sequence 5′-TGGAGATAACACTCTAAGCATAACTAAAGGT-3′ (SEQ ID NO:1); and/or (b) said second ABL1 primer comprises (i) at least 10 nucleotides of the sequence 5′-GATGTAGTTGCTTGGGACCCA-3′ (SEQ ID NO:2); or the sequence 5′-GATGTAGTTGCTTGGGACCCA-3′ (SEQ ID NO:2).

16-18. (canceled)

19. The method of claim 13, wherein said method comprises detecting the nucleic acid encoding ABL1 using an ABL1 probe comprising (i) at least about 10 nucleotides of the sequence 5′-CCATTTTTGGTTTGGGCTTCACACCATT-3′ (SEQ ID NO:5); or the sequence 5′-CCATTTTTGGTTTGGGCTTCACACCATT-3′ (SEQ ID NO:5).

20-21. (canceled)

22. The method of claim 1, further comprising measuring the level of expression of at least one additional prognostic marker gene in said biological sample.

23. The method of claim 1, wherein said biological sample comprises nucleic acids obtained from peripheral blood cells or bone marrow cells from said subject.

24. (canceled)

25. The method of claim 1, wherein said AML is an intermediate-risk AML.

26. The method of claim 25, wherein said intermediate-risk is FLT3-ITD negative AML.

27. A method for determining the likelihood that a subject suffers from EVI1-rearranged acute myeloid leukemia (EVI1-r AML), said method comprising:
determining the presence of one or more of the mutations depicted in FIG. 5A in a leukemia cell sample from said subject:
wherein the presence of said one or more mutations is indicative that said subject has a high likelihood of suffering from EVI1-r AML, and wherein the absence of said one or more mutations is indicative that said subject has a low likelihood of suffering from EVI1-r AML.

28. (canceled)

29. The method of claim 28, wherein said one or more mutations is:
a G to C or G to D substitution at a position corresponding to amino acid 12 of NRAS;
a G to D substitution at a position corresponding to amino acid 13 of NRAS;
a Q to K substitution at a position corresponding to amino acid 61 of NRAS;
a G to D substitution at a position corresponding to amino acid 12 KRAS;
a D to V substitution at a position corresponding to amino acid 61 of PTPN11;
an E to K substitution at a position corresponding to amino acid 69 of PTPN11;
an A to V substitution at a position corresponding to amino acids 72 of PTPN11;
a V to D substitution at a position corresponding to amino acids 1419 of NF1;
a mutation causing a frameshift at a position corresponding to amino acids 2423 of NF1;
an N to S substitution at a position corresponding to amino acid 159 of IKZF1;
an R to STOP substitution at a position corresponding to amino acid 213 of IKZF1;
a mutation causing a frameshift at a position corresponding to amino acid 270 of IKZF1;
an R to C substitution at a position corresponding to amino acid 625 of SF3B1;
a K to E substitution at a position corresponding to amino acid 700 of SF3B1;
a G to E substitution at a position corresponding to amino acid 740 of SF3B1;
a Q to R substitution at a position corresponding to amino acid 157 of U2AF1;
a Q to P substitution at a position corresponding to amino acid 157 of U2AF1;
an Y to C substitution at a position corresponding to amino acid 205 of TP53;
an R to W substitution at a position corresponding to amino acid 248 of TP53;
a mutation causing a frameshift at a position corresponding to amino acid 643 of ASXL1;
an R to C substitution at a position corresponding to amino acid 882 of DNMT3A;
an I to F substitution at a position corresponding to amino acid 406 of ETV6; and/or
a D to V substitution at a position corresponding to amino acid 816 of KIT.

30-46. (canceled)

47. A method for determining the likelihood that a subject suffers from EVI1-rearranged acute myeloid leukemia (EVI1-r AML), said method comprising:
determining the level of expression of at least one of the genes depicted in Table 2 in a leukemia cell sample from said subject:
TABLE 2

| | Genbank or RefSeq accession | | |
|---|---|---|---|
| Gene | Protein/cDNA/genomic (SEQ ID | Gene | Genbank or RefSeq accession |
| name | NO:) | name | Protein/cDNA/genomic |
| MECOM | NP_001098547.3/NM_001105077/ | ZNF385D | NP_078973.1/NM_024697.2/ |
| isoform | Gene ID: 2122 (SEQ ID NO: 19-20) | | Gene ID: 79750 (SEQ ID NO: 39-40) |
| 1d | | | |
| MECOM | NP_001157472.1/NM_001164000/ | SLC44A3 | NP_001107578.1/NM_001114106.2/ |
| isoform | Gene ID: 2122 (SEQ ID NO: 21-22) | | Gene ID: 126969 (SEQ ID NO: 41-42) |
| 1a | | | |
| VIP | NP_003372.1/NM_003381.3/ | GJA1 | NP_000156.1/NM_000165.3/ |
| | Gene ID: 743222 (SEQ ID NO: 23-24) | | NG_058308.1 (SEQ ID NO: 43-44) |
| PREX2 | NP_079146.2/NM_024870.2/ | CHRDL1 | NP_001137453.1/NM_001143981.1/ |
| | Gene ID: 80243 (SEQ ID NO: 25-26) | | NG_012816.1 (SEQ ID NO: 45-46) |
| MYCT1 | NP_079383.2/NM_025107.2/ | PRSS3P2 | NR_001296.3/Gene ID: 154754 |
| | Gene ID: 80177 (SEQ ID NO: 27-28) | | (SEQ ID NO: 47) |
| PAWR | NP_002574.2/NM_002583.2/ | DDIT4L | NP_660287.1/NM_145244.3/ |
| | Gene ID: 5074 (SEQ ID NO: 7-8) | | Gene ID: 115265 (SEQ ID NO: 48-49) |
| PRSS1 | NP_002760.1/NM_002769.4/ | GPC6 | NP_005699.1/NM_005708.3/Gene |
| | NG_058307.2 (SEQ ID NO: 29-30) | | ID: 10082 (SEQ ID NO: 50-51) |
| PRSS2 | NP_002761.1/NM_002770.2/ | LINC00989 | NR_038826.1/Gene ID: 100506035 |
| | NG_058322.1 (SEQ ID NO: 31-32) | | (SEQ ID NO: 52) |
| RFPL4A | NP_001138486.1/ | TUBAL3 | NP_079079.1/NM_024803.2/Gene |
| | NM_001145014.1/Gene ID: | | ID: 79861 (SEQ ID NO: 53-54) |
| | 342931 (SEQ ID NO: 33-34) | | |
| B3GNT3 | NP-055071.2/NM_014256.3/Gene | RIPK4 | NP_065690.2/NM_020639.2/Gene |
| | ID: 10331 (SEQ ID NO: 35-36) | | ID: 54101 (SEQ ID NO: 55-56) |
| TMEM40 | NP_001271335.1/ | MECOM | SEQ ID NO: 9 |
| | NM_001284406.1/Gene ID: 55287 | isoform 5 | |
| | (SEQ ID NO: 37-38) | | |
| MECOM | SEQ ID NO: 10 | MECOM | SEQ ID NO: 11 |
| isoform | | isoform 20 | |
| 16 | | | |
| MECOM | SEQ ID NO: 12 | MECOM | SEQ ID NO: 13 |
| isoform | | isoform 22 | |
| 21 | | | |
| MECOM | SEQ ID NO: 14 | MECOM | SEQ ID NO: 15 |
| isoform | | isoform 29 | |
| 28 | | | |
| MYCT1 | SEQ ID NO: 16 | LRBA | SEQ ID NO: 17 |
| isoform 6 | | isoform 3 | |
| LRBA | SEQ ID NO: 18 | | |
| isoform 6 | | | |

wherein a higher expression of said at least one genes in said sample relative to a control non-EVI1-r AML sample, is indicative that said subject has a high likelihood of suffering from EVI1-r AML.

48. The method of claim 47, wherein said method comprises determining the level of expression of at least one of a MECOM isoform, VIP, PREX2, MYCT1 and PAWR.

49-53. (canceled)


SEQUENCE LISTING (56 sequences)

SEQ ID NO: 1 - DNA, 31 bp, Artificial Sequence
  Synthetic oligonucleotide
tggagataacactctaagcataactaaaggt

SEQ ID NO: 2 - DNA, 21 bp, Artificial Sequence
  Synthetic oligonucleotide
gatgtagttgcttgggaccca

SEQ ID NO: 3 - DNA, 18 bp, Artificial Sequence
  Synthetic oligonucleotide
tggtcaacatccctgccg

SEQ ID NO: 4 - DNA, 20 bp, Artificial Sequence
  Synthetic oligonucleotide
ttgcatcttctcgtttccgc

SEQ ID NO: 5 - DNA, 28 bp, Artificial Sequence
  Synthetic oligonucleotide
ccatttttggtttgggcttcacaccatt

SEQ ID NO: 6 - DNA, 24 bp, Artificial Sequence
  Synthetic oligonucleotide
agtacgaagatgatgaagcagggc

SEQ ID NO: 7 - DNA, 1967 bp, Homo sapiens
  CDS (287)..(1309)
ctgtcctgggattgcctggagctccgcaccgcgagtttgccgcggcactttccgcgcggcggaagagcgcgcgccagcttcggcacacctgggagccggatcccagccctacgcctcgtcccctacaagctcctccaagccccgccggctgctgtgggagcggcggccgtccctctcctggaggtcgtctcctggcatcctcggggccgcaggaaggaagaggaggcagcggccggagccctggtgggcggcctgaggtgagagcccgaccggcccctttgggaatatggcgaccggtggctaccggaccagcagcggcctcggcggcagcaccacagacttcctggaggagtggaaggcgaaacgcgagaagatgcgcgccaagcagaaccccccgggcccggcccccccgggagggggcagcagcgacgccgctgggaagccccccgcgggggctctgggcaccccggcggccgccgctgccaacgagctcaacaacaacctcccgggcggcgcgccggccgcacctgccgtccccggtcccgggggcgtgaactgcgcggtcggctccgccatgctgacgcgggcggcccccggcccgcggcggtcggaggacgagcccccagccgcctctgcctcggctgcaccgccgccccagcgtgacgaggaggagccggacggcgtcccagagaagggcaagagctcgggccccagtgccaggaaaggcaaggggcagatcgagaagaggaagctgcgggagaagcggcgctccaccggcgtggtcaacatccctgccgcagagtgcttagatgagtacgaagatgatgaagcagggcagaaagagcggaaacgagaagatgcaattacacaacagaacactattcagaatgaagctgtaaacttactagatccaggcagttcctatctgctacaggagccacctagaacagtttcaggcagatataaaagcacaaccagtgtctctgaagaagatgtctcaagtagatattctcgaacagatagaagtgggttccctagatataacagggatgcaaatgtttcaggtactctggtttcaagtagcacactggaaaagaaaattgaagatcttgaaaaggaagtagtaagagaaagacaagaaaacctaagacttgtgagactgatgcaagataaagaggaaatgattggaaaactcaaagaagaaattgatttattaaatagagacctagatgacatagaagatgaaaatgaacagctaaagcaggaaaataaaactcttttgaaagttgtgggtcagctgaccaggtagaggattcaagactcaatgtggaaaaaatattttaaactactgattgaatgttaatggtcaatgctagcacaatattcctatgctgcaatacattaaaataactaagcaagtatatttatttctagcaaacagatgtttgttttcaaaatacttctttttcattattggttttaaaaaagcattatccttttatctcacaaataagtaatatctttcagttattaaatgatagataatgcctttttggttttgtgtggtattcaactaatacatggtttaaagtcacagccgtttgaatatattttatcttggtagtacattttctcccttaggaatatacatagtctttgtttacatgagttcaaatacttttgggatgttaccttcacatgtcctattactgatgtgtgcaaccttttatgtgttgatgactcactcataaaggtttttgtctactgtcatttgttctttccacttattctaagcatttagagtaatagagtcatacttttttataacagcaaccttttaaaaggaaagctcttataaagtcactgtcatgttttagttgactaaatataaatttaagagaatacttgaattgtgctatagtaaataaaaatttactattttgtgtttgaaaaaaaaaaaaaaaaaa

SEQ ID NO: 8 - protein, 340 aa, Homo sapiens
MATGGYRTSSGLGGSTTDFLEEWKAKREKMRAKQNPPGPAPPGGGSSDAAGKPPAGALGTPAAAAANELNNNLPGGAPAAPAVPGPGGVNCAVGSAMLTRAAPGPRRSEDEPPAASASAAPPPQRDEEEPDGVPEKGKSSGPSARKGKGQIEKRKLREKRRSTGVVNIPAAECLDEYEDDEAGQKERKREDAITQQNTIQNEAVNLLDPGSSYLLQEPPRTVSGRYKSTTSVSEEDVSSRYSRTDRSGFPRYNRDANVSGTLVSSSTLEKKIEDLEKEVVRERQENLRLVRLMQDKEEMIGKLKEEIDLLNRDLDDIEDENEQLKQENKTLLKVVGQLTR

SEQ ID NO: 9 - DNA, 4057 bp, Homo sapiens
aaagtttgtcacttcggacagtttcctgccggggcaccgtttctacgggcgccccccgcccccagccttctttcctcctcgcccgcagtctcgcggagccctgctgcttatctacgttgctaagccgggcgatttccttgttcctcctgcgaaacggtgcggtctggacacgtctccggggtgggtcgtccggccttcgatcttagacgaattttacaatgtgaagttctgcatagatgccagtcaaccagatgttggaagctggctcaagtacattagattcgctggctgttatgatcagcacaaccttgttgcatgccagataaatgatcagatattctatagagtagttgcagacattgcgccgggagaggagcttctgctgttcatgaagagcgaagactatccccatgaaactatggcgccggatatccacgaagaacggcaatatcgctgcgaagactgtgaccagctctttgaatctaaggctgaactagcagatcaccaaaagtttccatgcagtactcctcactcagcattttcaatggttgaagaggactttcagcaaaaactcgaaagcgagaatgatctccaagagatacacacgatccaggagtgtaaggaatgtgaccaagtttttcctgatttgcaaagcctggagaaacacatgctgtcacatactgaagagagggaatacaagtgtgatcagtgtcccaaggcatttaactggaagtccaatttaattcgccaccagatgtcacatgacagtggaaagcactatgaatgtgaaaactgtgccaagcaggttttcacggaccctagcaaccttcagcggcacattcgctctcagcatgtcggtgcccgggcccatgcatgcccggagtgtggcaaaacgtttgccacttcgtcgggcctcaaacaacacaagcacatccacagcagtgtgaagccctttatctcattctctcaatcaatgtacccatttcctgatagagacttgagatcgttacctttgaaaatggaaccccaatcaccaggtgaagtaaagaaactgcagaagggcagctctgagtccccctttgatctcaccactaagcgaaaggatgagaagcccttgactccagtcccctccaagcctccagtgacacctgccacaagccaagaccagcccctggatctaagtatgggcagtaggagtagagccagtgggacaaagctgactgagcctcgaaaaaaccacgtgtttgggggaaaaaaaggaagcaacgtcgaatcaagacctgcttcagatggttccttgcagcatgcaagacccactcctttctttatggaccctatttacagagtagagaaaagaaaactaactgacccacttgaagctttaaaagagaaatacttgaggccttctccaggattcttgtttcacccacaattccaactgcctgatcagagaacttggatgtcagctattgaaaacatggcagaaaagctagagagcttcagtgccctgaaacctgaggccagtgagctcttacagtcagtgccctctatgttcaacttcagggcgcctcccaatgccctgccagagaaccttctgcggaagggaaaggagcgctatacctgcagatactgtggcaagatttttccaaggtctgcaaacctaacacggcacttgagaacccacacaggagagcagccttacagatgcaaatactgtgacagatcatttagcatatcttctaacttgcaaaggcatgttcgcaacatccacaataaagagaagccatttaagtgtcacttatgtgataggtgttttggtcaacaaaccaatttagacagacacctaaagaaacatgagaatgggaacatgtccggtacagcaacatcgtcgcctcattctgaactggaaagtacaggtgcgattctggatgacaaagaagatgcttacttcacagaaattcgaaatttcattgggaacagcaaccatggcagccaatctcccaggaatgtggaggagagaatgaatggcagtcattttaaagatgaaaaggctttggtgaccagtcaaaattcagacttgctggatgatgaagaagttgaagatgaggtgttgttagatgaggaggatgaagacaatgatattactggaaaaacaggaaaggaaccagtgacaagtaatttacatgaaggaaaccctgaggatgactatgaagaaaccagtgccctggagatgagttgcaagacatccccagtgaggtataaagaggaagaatataaaagtggactttctgctctagatcatataaggcacttcacagatagcctcaaaatgaggaaaatggaagataatcaatattctgaagctgagctgtcttcttttagtacttcccatgtgccagaggaacttaagcagccgttacacagaaagtccaaatcgcaggcatatgctatgatgctgtcactgtctgacaaggagtccctccattctacatcccacagttcttccaacgtgtggcacagtatggccagggctgcggcggaatccagtgctatccagtccataagccacgtatgacgttatcaaggttgaccagagtgggaccaagtccaacagtagcatggctctttcatataggactatttacaagactgctgagcagaatgccttataaacctgcagggtcactcatctaaagtctagtgaccttaaactgaatgatttaaaaaagaaaagaaagaaaaaagaaactatttattctcgatattttgttttgcacagcaaaggcagctgctgacttctggaagatcaatcaatgcgacttaaagtgattcagtgaaaacaaaaaacttggtgggctgaaggcatcttccagtttaccccaccttagggtatgggtgggtgagaagggcagttgagatggcagcattgatatgaatgaacactccatagaaactgaattctcttttgtacaagatcacctgacatgattgggaacagttgcttttaattacagatttaatttttttcttcgttaaagttttatgtaatttaaccctttgaagacagaagtagttggatgaaatgcacagtcaattattatagaaactgataacagggagtacttgttcccccttttgccttcttaagtacattgtttaaaactagggaaaaagggtatgtgtatattgtaaactatggatgttaacactcaaagaggttaagtcagtgaagtaacctattcatcaccagtaccgctgtaccactaataaattgtttgccaaatccttgtaataacatcttaattttagacaatcatgtcactgtttttaatgtttatttttttgtgtgtgttgcgtgtatcatgtatttatttgttggcaaactattgtttgttgattaaaatagcactgttccagtcagccactactttatgacgtctgaggcacacccctttccgaatttcaaggaccaaggtgacccgacctgtgtatgagagtgccaaatggtgtttggcttttcttaacattcctttttgtttgtttgttttgttttccttcttaatgaactaaatacgaatagatgcaacttagtttttgtaatactgaaatcgattcaattgtataaacgattataatttctttcatggaagcatgattcttctgattaaaaactgtactccatattttatgctggttgtctgcaagcttgtgcgatgttatgttcatgttaatcctatttgtaaaatgaagtgttcccaaccttatgttaaaagagagaagtaaataacagactgtattcagttattttgccctttattgaggaaccagatttgttttctttttgtttgtaatctcattttgaaataatcagcaagttgaggtactttcttcaaatgctttgtacaatataaactgttatgcctttcagtgcattactatgggaggagcaactaaaaaataaagacttacaaaaaggagtatttttccccactgtgtggtatttttccttcatgcaattttactttccctttgggatccaatgatttaaagatatgat

SEQ ID NO: 10 - DNA, 5907 bp, Homo sapiens
cacactcgcgcgcacacacacacacacacaccacacttgtgctttcaagacatcgaaacggaggctatttccctggggaaagaaatcctgcctggcgagatctccccattggttgtttacccggagaaatctacatgtttaagggggatggtgcatccataatcagtctgtccctataggacttgggtcttggcgacctttttgtgacctctcccgccagaggaggctgctgtcactttaaaaatttaaaagaggagcccgtctggcttccgatcagatcactctgggcggcgggagatagctccctttctccctcgccccgggttctttctggatggccgagcagatcctctttaaagagacagttcatgaaatagaaacccggcggctgagcttggagttgcgaaaggggacgatcccgtgagggtccaggacccgcgaaggcgctgcggaggatctgaaagggggatagagctcccctcgcctccccaggccccccaccttttcaaactctcctcctcctgcttgttttcccccattggaactgggaaggagaagtagaagttttagtgggtttcagataactttcattacacatcgggctgataagagcaagagaaagtgagaaaagagggaggtgatgtgaaccagaaggaatagctccgagctcatttaggaagggggaaaaagccaaaacacaccaaacccgggtcacccagacgaaagaagacttcatttcttgtattaaaaatacactgttggcggacaataaatccgaaacgcgtggtcctggagagcagatcctagagacggacaaagttgtcagagacccatttggaaatcgagacgcgaggcttttaaaaaattattattattatttttaaacatctctaaatgttgctcgggatcgtttgaaaggattttcgtgcaggagcgttgggggctgctgattattttattttgtttattttgattcttctgtgaatgcctattattgctgagttgaggccatagaaatctaaagatcttagacgaattttacaatgtgaagttctgcatagatgccagtcaaccagatgttggaagctggctcaagtacattagattcgctggctgttatgatcagcacaaccttgttgcatgccagataaatgatcagatattctatagagtagttgcagacattgcgccgggagaggagcttctgctgttcatgaagagcgaagactatccccatgaaactatggcgccggatatccacgaagaacggcaatatcgctgcgaagactgtgaccagctctttgaatctaaggctgaactagcagatcaccaaaagtttccatgcagtactcctcactcagcattttcaatggttgaagaggactttcagcaaaaactcgaaagcgagaatgatctccaagagatacacacgatccaggagtgtaaggaatgtgaccaagtttttcctgatttgcaaagcctggagaaacacatgctgtcacatactgaagagagggaatacaagtgtgatcagtgtcccaaggcatttaactggaagtccaatttaattcgccaccagatgtcacatgacagtggaaagcactatgaatgtgaaaactgtgccaagcaggttttcacggaccctagcaaccttcagcggcacattcgctctcagcatgtcggtgcccgggcccatgcatgcccggagtgtggcaaaacgtttgccacttcgtcgggcctcaaacaacacaagcacatccacagcagtgtgaagccctttatctgtgaggtctgccataaatcctatactcagttttcaaacctttgccgtcataagcgcatgcatgctgattgcagaacccaaatcaagtgcaaagactgtggacaaatgttcagcactacgtcttccttaaataaacacaggaggttttgtgagggcaagaaccattttgcggcaggtggattttttggccaaggcatttcacttcctggaaccccagctatggataaaacgtccatggttaatatgagtcatgccaacccgggccttgctgactattttggcgccaataggcatcctgctggtcttacctttccaacagctcctggattttcttttagcttccctggtctgtttccttccggcttgtaccacaggcctcctttgatacctgctagttctcctgttaaaggactatcaagtactgaacagacaaacaaaagtcaaagtcccctcatgacacatcctcagatactgccagctacacaggatattttgaaggcactatctaaacacccatctgtaggggacaataagccagtggagctccagcccgagaggtcctctgaagagaggccctttgagaaaatcagtgaccagtcagagagtagtgaccttgatgatgtcagtacaccaagtggcagtgacctggaaacaacctcgggctctgatctggaaagtgacattgaaagtgataaagagaaatttaaagaaaatggtaaaatgttcaaagacaaagtaagccctcttcagaatctggcttcaataaataataagaaagaatacagcaatcattccattttctcaccatctttagaggagcagactgcggtgtcaggagctgtgaatgattctataaaggctattgcttctattgctgaaaaatactttggttcaacaggactggtggggctgcaagacaaaaaagttggagctttaccttacccttccatgtttcccctcccattttttccagcattctctcaatcaatgtacccatttcctgatagagacttgagatcgttacctttgaaaatggaaccccaatcaccaggtgaagtaaagaaactgcagaagggcagctctgagtccccctttgatctcaccactaagcgaaaggatgagaagcccttgactccagtcccctccaagcctccagtgacacctgccacaagccaagaccagcccctggatctaagtatgggcagtaggagtagagccagtgggacaaagctgactgagcctcgaaaaaaccacgtgtttgggggaaaaaaaggaagcaacgtcgaatcaagacctgcttcagatggttccttgcagcatgcaagacccactcctttctttatggaccctatttacagagtagagaaaagaaaactaactgacccacttgaagctttaaaagagaaatacttgaggccttctccaggattcttgtttcacccacaattccaactgcctgatcagagaacttggatgtcagctattgaaaacatggcagaaaagctagagagcttcagtgccctgaaacctgaggccagtgagctcttacagtcagtgccctctatgttcaacttcagggcgcctcccaatgccctgccagagaaccttctgcggaagggaaaggagcgctatacctgcagatactgtggcaagatttttccaaggtctgcaaacctaacacggcacttgagaacccacacaggagagcagccttacagatgcaaatactgtgacagatcatttagcatatcttctaacttgcaaaggcatgttcgcaacatccacaataaagagaagccatttaagtgtcacttatgtgataggtgttttggtcaacaaaccaatttagacagacacctaaagaaacatgagaatgggaacatgtccggtacagcaacatcgtcgcctcattctgaactggaaagtacaggtgcgattctggatgacaaagaagatgcttacttcacagaaattcgaaatttcattgggaacagcaaccatggcagccaatctcccaggaatgtggaggagagaatgaatggcagtcattttaaagatgaaaaggctttggtgaccagtcaaaattcagacttgctggatgatgaagaagttgaagatgaggtgttgttagatgaggaggatgaagacaatgatattactggaaaaacaggaaaggaaccagtgacaagtaatttacatgaaggaaaccctgaggatgactatgaagaaaccagtgccctggagatgagttgcaagacatccccagtgaggtataaagaggaagaatataaaagtggactttctgctctagatcatataaggcacttcacagatagcctcaaaatgaggaaaatggaagataatcaatattctgaagctgagctgtcttcttttagtacttcccatgtgccagaggaacttaagcagccgttacacagaaagtccaaatcgcaggaggaaagtaaagcttaaaaggttaagcaacttgctcaagatcacacagctaggcatatgctatgatgctgtcactgtctgacaaggagtccctccattctacatcccacagttcttccaacgtgtggcacagtatggccagggctgcggcggaatccagtgctatccagtccataagccacgtatgacgttatcaaggttgaccagagtgggaccaagtccaacagtagcatggctctttcatataggactatttacaagactgctgagcagaatgccttataaacctgcagggtcactcatctaaagtctagtgaccttaaactgaatgatttaaaaaagaaaagaaagaaaaaagaaactatttattctcgatattttgttttgcacagcaaaggcagctgctgacttctggaagatcaatcaatgcgacttaaagtgattcagtgaaaacaaaaaacttggtgggctgaaggcatcttccagtttaccccaccttagggtatgggtgggtgagaagggcagttgagatggcagcattgatatgaatgaacactccatagaaactgaattctcttttgtacaagatcacctgacatgattgggaacagttgcttttaattacagatttaatttttttcttcgttaaagttttatgtaatttaaccctttgaagacagaagtagttggatgaaatgcacagtcaattattatagaaactgataacagggagtacttgttcccccttttgccttcttaagtacattgtttaaaactagggaaaaagggtatgtgtatattgtaaactatggatgttaacactcaaagaggttaagtcagtgaagtaacctattcatcaccagtaccgctgtaccactaataaattgtttgccaaatccttgtaataacatcttaattttagacaatcatgtcactgtttttaatgtttatttttttgtgtgtgttgcgtgtatcatgtatttatttgttggcaaactattgtttgttgattaaaatagcactgttccagtcagccactactttatgacgtctgaggcacacccctttccgaatttcaaggaccaaggtgacccgacctgtgtatgagagtgccaaatggtgtttggcttttcttaacattcctttttgtttgtttgttttgttttccttcttaatgaactaaatacgaatagatgcaacttagtttttgtaatactgaaatcgattcaattgtataaacgattataatttctttcatggaagcatgattcttctgattaaaaactgtactccatattttatgctggttgtctgcaagcttgtgcgatgttatgttcatgttaatcctatttgtaaaatgaagtgttcccaaccttatgttaaaagagagaagtaaataacagactgtattcagttattttgccctttattgaggaaccagatttgttttctttttgtttgtaatctcattttgaaataatcagcaagttgaggtactttcttcaaatgctttgtacaatataaactgttatgcctttcagtgcattactatgggaggagcaactaaaaaataaagacttacaaaaaggagtatttttccccactgtgtggtatttttccttcatgcaattttactttccctttgggatccaatgatttaaagatatgat

SEQ ID NO: 11 - DNA, 4900 bp, Homo sapiens
ccttgccaagtaacagctttgctgtccaacatcgtgtgctgcttcgcgagaaagtcacattcggaccctttggctagattgcttattcatagggcttcttgactaaagcccttggagcactgggtttttcttgaagtatatgatcttagacgaattttacaatgtgaagttctgcatagatgccagtcaaccagatgttggaagctggctcaagtacattagattcgctggctgttatgatcagcacaaccttgttgcatgccagataaatgatcagatattctatagagtagttgcagacattgcgccgggagaggagcttctgctgttcatgaagagcgaagactatccccatgaaactatggcgccggatatccacgaagaacggcaatatcgctgcgaagactgtgaccagctctttgaatctaaggctgaactagcagatcaccaaaagtttccatgcagtactcctcactcagcattttcaatggttgaagaggactttcagcaaaaactcgaaagcgagaatgatctccaagagatacacacgatccaggagtgtaaggaatgtgaccaagtttttcctgatttgcaaagcctggagaaacacatgctgtcacatactgaagagagggaatacaagtgtgatcagtgtcccaaggcatttaactggaagtccaatttaattcgccaccagatgtcacatgacagtggaaagcactatgaatgtgaaaactgtgccaagcaggttttcacggaccctagcaaccttcagcggcacattcgctctcagcatgtcggtgcccgggcccatgcatgcccggagtgtggcaaaacgtttgccacttcgtcgggcctcaaacaacacaagcacatccacagcagtgtgaagccctttatctgtgaggtctgccataaatcctatactcagttttcaaacctttgccgtcataagcgcatgcatgctgattgcagaacccaaatcaagtgcaaagactgtggacaaatgttcagcactacgtcttccttaaataaacacaggaggttttgtgagggcaagaaccattttgcggcaggtggattttttggccaaggcatttcacttcctggaaccccagctatggataaaacgtccatggttaatatgagtcatgccaacccgggccttgctgactattttggcgccaataggcatcctgctggtcttacctttccaacagctcctggattttcttttagcttccctggtctgtttccttccggcttgtaccacaggcctcctttgatacctgctagttctcctgttaaaggactatcaagtactgaacagacaaacaaaagtcaaagtcccctcatgacacatcctcagatactgccagctacacaggatattttgaaggcactatctaaacacccatctgtaggggacaataagccagtggagctccagcccgagaggtcctctgaagagaggccctttgagaaaatcagtgaccagtcagagagtagtgaccttgatgatgtcagtacaccaagtggcagtgacctggaaacaacctcgggctctgatctggaaagtgacattgaaagtgataaagagaaatttaaagaaaatggtaaaatgttcaaagacaaagtaagccctcttcagaatctggcttcaataaataataagaaagaatacagcaatcattccattttctcaccatctttagaggagcagactgcggtgtcaggagctgtgaatgattctataaaggctattgcttctattgctgaaaaatactttggttcaacaggactggtggggctgcaagacaaaaaagttggagctttaccttacccttccatgtttcccctcccattttttccagcattctctcaatcaatgtacccatttcctgatagagacttgagatcgttacctttgaaaatggaaccccaatcaccaggtgaagtaaagaaactgcagaagggcagctctgagtccccctttgatctcaccactaagcgaaaggatgagaagcccttgactccagtcccctccaagcctccagtgacacctgccacaagccaagaccagcccctggatctaagtatgggcagtaggagtagagccagtgggacaaagctgactgagcctcgaaaaaaccacgtgtttgggggaaaaaaaggaagcaacgtcgaatcaagacctgcttcagatggttccttgcagcatgcaagacccactcctttctttatggaccctatttacagagtagagaaaagaaaactaactgacccacttgaagctttaaaagagaaatacttgaggccttctccaggattcttgtttcacccacaattccaactgcctgatcagagaacttggatgtcagctattgaaaacatggcagaaaagctagagagcttcagtgccctgaaacctgaggccagtgagctcttacagtcagtgccctctatgttcaacttcagggcgcctcccaatgccctgccagagaaccttctgcggaagggaaaggagcgctatacctgcagatactgtggcaagatttttccaaggtctgcaaacctaacacggcacttgagaacccacacaggagagcagccttacagatgcaaatactgtgacagatcatttagcatatcttctaacttgcaaaggcatgttcgcaacatccacaataaagagaagccatttaagtgtcacttatgtgataggtgttttggtcaacaaaccaatttagacagacacctaaagaaacatgagaatgggaacatgtccggtacagcaacatcgtcgcctcattctgaactggaaagtacaggtgcgattctggatgacaaagaagatgcttacttcacagaaattcgaaatttcattgggaacagcaaccatggcagccaatctcccaggaatgtggaggagagaatgaatggcagtcattttaaagatgaaaaggctttggtgaccagtcaaaattcagacttgctggatgatgaagaagttgaagatgaggtgttgttagatgaggaggatgaagacaatgatattactggaaaaacaggaaaggaaccagtgacaagtaatttacatgaaggaaaccctgaggatgactatgaagaaaccagtgccctggagatgagttgcaagacatccccagtgaggtataaagaggaagaatataaaagtggactttctgctctagatcatataaggcacttcacagatagcctcaaaatgaggaaaatggaagataatcaatattctgaagctgagctgtcttcttttagtacttcccatgtgccagaggaacttaagcagccgttacacagaaagtccaaatcgcaggcatatgctatgatgctgtcactgtctgacaaggagtccctccattctacatcccacagttcttccaacgtgtggcacagtatggccagggctgcggcggaatccagtgctatccagtccataagccacgtatgacgttatcaaggttgaccagagtgggaccaagtccaacagtagcatggctctttcatataggactatttacaagactgctgagcagaatgccttataaacctgcagggtcactcatctaaagtctagtgaccttaaactgaatgatttaaaaaagaaaagaaagaaaaaagaaactatttattctcgatattttgttttgcacagcaaaggcagctgctgacttctggaagatcaatcaatgcgacttaaagtgattcagtgaaaacaaaaaacttggtgggctgaaggcatcttccagtttaccccaccttagggtatgggtgggtgagaagggcagttgagatggcagcattgatatgaatgaacactccatagaaactgaattctcttttgtacaagatcacctgacatgattgggaacagttgcttttaattacagatttaatttttttcttcgttaaagttttatgtaatttaaccctttgaagacagaagtagttggatgaaatgcacagtcaattattatagaaactgataacagggagtacttgttcccccttttgccttcttaagtacattgtttaaaactagggaaaaagggtatgtgtatattgtaaactatggatgttaacactcaaagaggttaagtcagtgaagtaacctattcatcaccagtaccgctgtaccactaataaattgtttgccaaatccttgtaataacatcttaattttagacaatcatgtcactgtttttaatgtttatttttttgtgtgtgttgcgtgtatcatgtatttatttgttggcaaactattgtttgttgattaaaatagcactgttccagtcagccactactttatgacgtctgaggcacacccctttccgaatttcaaggaccaaggtgacccgacctgtgtatgagagtgccaaatggtgtttggcttttcttaacattcctttttgtttgtttgttttgttttccttcttaatgaactaaatacgaatagatgcaacttagtttttgtaatactgaaatcgattcaattgtataaacgattataatttctttcatggaagcatgattcttctgattaaaaactgtactccatattttatgctggttgtctgcaagcttgtgcgatgttatgttcatgttaatcctatttgtaaaatgaagtgttcccaaccttatgttaaaagagagaagtaaataacagactgtattcagttattttgccctttattgaggaaccagatttgttttctttttgtttgtaatctcattttgaaataatcagcaagttgaggtactttcttcaaatgctttgtacaatataaactgttatgcctttcagtgcattactatgggaggagcaactaaaaaataaagacttacaaaaaggagtattttt

SEQ ID NO: 12 - DNA, 4835 bp, Homo sapiens
ccttgccaagtaacagctttgctgtccaacatcgtgtgctgcttcgcgagaaagtcacattcggaccctttggctagattatcttagacgaattttacaatgtgaagttctgcatagatgccagtcaaccagatgttggaagctggctcaagtacattagattcgctggctgttatgatcagcacaaccttgttgcatgccagataaatgatcagatattctatagagtagttgcagacattgcgccgggagaggagcttctgctgttcatgaagagcgaagactatccccatgaaactatggcgccggatatccacgaagaacggcaatatcgctgcgaagactgtgaccagctctttgaatctaaggctgaactagcagatcaccaaaagtttccatgcagtactcctcactcagcattttcaatggttgaagaggactttcagcaaaaactcgaaagcgagaatgatctccaagagatacacacgatccaggagtgtaaggaatgtgaccaagtttttcctgatttgcaaagcctggagaaacacatgctgtcacatactgaagagagggaatacaagtgtgatcagtgtcccaaggcatttaactggaagtccaatttaattcgccaccagatgtcacatgacagtggaaagcactatgaatgtgaaaactgtgccaaggttttcacggaccctagcaaccttcagcggcacattcgctctcagcatgtcggtgcccgggcccatgcatgcccggagtgtggcaaaacgtttgccacttcgtcgggcctcaaacaacacaagcacatccacagcagtgtgaagccctttatctgtgaggtctgccataaatcctatactcagttttcaaacctttgccgtcataagcgcatgcatgctgattgcagaacccaaatcaagtgcaaagactgtggacaaatgttcagcactacgtcttccttaaataaacacaggaggttttgtgagggcaagaaccattttgcggcaggtggattttttggccaaggcatttcacttcctggaaccccagctatggataaaacgtccatggttaatatgagtcatgccaacccgggccttgctgactattttggcgccaataggcatcctgctggtcttacctttccaacagctcctggattttcttttagcttccctggtctgtttccttccggcttgtaccacaggcctcctttgatacctgctagttctcctgttaaaggactatcaagtactgaacagacaaacaaaagtcaaagtcccctcatgacacatcctcagatactgccagctacacaggatattttgaaggcactatctaaacacccatctgtaggggacaataagccagtggagctccagcccgagaggtcctctgaagagaggccctttgagaaaatcagtgaccagtcagagagtagtgaccttgatgatgtcagtacaccaagtggcagtgacctggaaacaacctcgggctctgatctggaaagtgacattgaaagtgataaagagaaatttaaagaaaatggtaaaatgttcaaagacaaagtaagccctcttcagaatctggcttcaataaataataagaaagaatacagcaatcattccattttctcaccatctttagaggagcagactgcggtgtcaggagctgtgaatgattctataaaggctattgcttctattgctgaaaaatactttggttcaacaggactggtggggctgcaagacaaaaaagttggagctttaccttacccttccatgtttcccctcccattttttccagcattctctcaatcaatgtacccatttcctgatagagacttgagatcgttacctttgaaaatggaaccccaatcaccaggtgaagtaaagaaactgcagaagggcagctctgagtccccctttgatctcaccactaagcgaaaggatgagaagcccttgactccagtcccctccaagcctccagtgacacctgccacaagccaagaccagcccctggatctaagtatgggcagtaggagtagagccagtgggacaaagctgactgagcctcgaaaaaaccacgtgtttgggggaaaaaaaggaagcaacgtcgaatcaagacctgcttcagatggttccttgcagcatgcaagacccactcctttctttatggaccctatttacagagtagagaaaagaaaactaactgacccacttgaagctttaaaagagaaatacttgaggccttctccaggattcttgtttcacccacaattccaactgcctgatcagagaacttggatgtcagctattgaaaacatggcagaaaagctagagagcttcagtgccctgaaacctgaggccagtgagctcttacagtcagtgccctctatgttcaacttcagggcgcctcccaatgccctgccagagaaccttctgcggaagggaaaggagcgctatacctgcagatactgtggcaagatttttccaaggtctgcaaacctaacacggcacttgagaacccacacaggagagcagccttacagatgcaaatactgtgacagatcatttagcatatcttctaacttgcaaaggcatgttcgcaacatccacaataaagagaagccatttaagtgtcacttatgtgataggtgttttggtcaacaaaccaatttagacagacacctaaagaaacatgagaatgggaacatgtccggtacagcaacatcgtcgcctcattctgaactggaaagtacaggtgcgattctggatgacaaagaagatgcttacttcacagaaattcgaaatttcattgggaacagcaaccatggcagccaatctcccaggaatgtggaggagagaatgaatggcagtcattttaaagatgaaaaggctttggtgaccagtcaaaattcagacttgctggatgatgaagaagttgaagatgaggtgttgttagatgaggaggatgaagacaatgatattactggaaaaacaggaaaggaaccagtgacaagtaatttacatgaaggaaaccctgaggatgactatgaagaaaccagtgccctggagatgagttgcaagacatccccagtgaggtataaagaggaagaatataaaagtggactttctgctctagatcatataaggcacttcacagatagcctcaaaatgaggaaaatggaagataatcaatattctgaagctgagctgtcttcttttagtacttcccatgtgccagaggaacttaagcagccgttacacagaaagtccaaatcgcaggcatatgctatgatgctgtcactgtctgacaaggagtccctccattctacatcccacagttcttccaacgtgtggcacagtatggccagggctgcggcggaatccagtgctatccagtccataagccacgtatgacgttatcaaggttgaccagagtgggaccaagtccaacagtagcatggctctttcatataggactatttacaagactgctgagcagaatgccttataaacctgcagggtcactcatctaaagtctagtgaccttaaactgaatgatttaaaaaagaaaagaaagaaaaaagaaactatttattctcgatattttgttttgcacagcaaaggcagctgctgacttctggaagatcaatcaatgcgacttaaagtgattcagtgaaaacaaaaaacttggtgggctgaaggcatcttccagtttaccccaccttagggtatgggtgggtgagaagggcagttgagatggcagcattgatatgaatgaacactccatagaaactgaattctcttttgtacaagatcacctgacatgattgggaacagttgcttttaattacagatttaatttttttcttcgttaaagttttatgtaatttaaccctttgaagacagaagtagttggatgaaatgcacagtcaattattatagaaactgataacagggagtacttgttcccccttttgccttcttaagtacattgtttaaaactagggaaaaagggtatgtgtatattgtaaactatggatgttaacactcaaagaggttaagtcagtgaagtaacctattcatcaccagtaccgctgtaccactaataaattgtttgccaaatccttgtaataacatcttaattttagacaatcatgtcactgtttttaatgtttatttttttgtgtgtgttgcgtgtatcatgtatttatttgttggcaaactattgtttgttgattaaaatagcactgttccagtcagccactactttatgacgtctgaggcacacccctttccgaatttcaaggaccaaggtgacccgacctgtgtatgagagtgccaaatggtgtttggcttttcttaacattcctttttgtttgtttgttttgttttccttcttaatgaactaaatacgaatagatgcaacttagtttttgtaatactgaaatcgattcaattgtataaacgattataatttctttcatggaagcatgattcttctgattaaaaactgtactccatattttatgctggttgtctgcaagcttgtgcgatgttatgttcatgttaatcctatttgtaaaatgaagtgttcccaaccttatgttaaaagagagaagtaaataacagactgtattcagttattttgccctttattgaggaaccagatttgttttctttttgtttgtaatctcattttgaaataatcagcaagttgaggtactttcttcaaatgctttgtacaatataaactgttatgcctttcagtgcattactatgggaggagcaactaaaaaataaagacttacaaaaaggagtattttt

SEQ ID NO: 13 - DNA, 5732 bp, Homo sapiens
cacacacacacacacacaccacacttgtgctttcaagacatcgaaacggaggctatttccctggggaaagaaatcctgcctggcgagatctccccattggttgtttacccggagaaatctacatgtttaagggggatggtgcatccataatcagtctgtccctataggacttgggtcttggcgacctttttgtgacctctcccgccagaggaggctgctgtcactttaaaaatttaaaagaggagcccgtctggcttccgatcagatcactctgggcggcgggagatagctccctttctccctcgccccgggttctttctggatggccgagcagatcctctttaaagagacagttcatgaaatagaaacccggcggctgagcttggagttgcgaaaggggacgatcccgtgagggtccaggacccgcgaaggcgctgcggaggatctgaaagggggatagagctcccctcgcctccccaggccccccaccttttcaaactctcctcctcctgcttgttttcccccattggaactgggaaggagaagtagaagttttagtgggtttcagataactttcattacacatcgggctgataagagcaagagaaagtgagaaaagagggaggtgatgtgaaccagaaggaatagctccgagctcatttaggaagggggaaaaagccaaaacacaccaaacccgggtcacccagacgaaagaagacttcatttcttgtattaaaaatacactgttggcggacaataaatccgaaacgcgtggtcctggagagcagatcctagagacggacaaagttgtcagagacccatttggaaatcgagacgcgaggcttttaaaaaattattattattatttttaaacatctctaaatgttgctcgggatcgtttgaaaggattttcgtgcaggagcgttgggggctgctgattattttattttgtttattttgattcttctgtgaatgcctattattgctgagttgaggccatagaaatctaaagatcttagacgaattttacaatgtgaagttctgcatagatgccagtcaaccagatgttggaagctggctcaagtacattagattcgctggctgttatgatcagcacaaccttgttgcatgccagataaatgatcagatattctatagagtagttgcagacattgcgccgggagaggagcttctgctgttcatgaagagcgaagactatccccatgaaactatggcgccggatatccacgaagaacggcaatatcgctgcgaagactgtgaccagctctttgaatctaaggctgaactagcagatcaccaaaagtttccatgcagtactcctcactcagcattttcaatggttgaagaggactttcagcaaaaactcgaaagcgagaatgatctccaagagatacacacgatccaggagtgtaaggaatgtgaccaagtttttcctgatttgcaaagcctggagaaacacatgctgtcacatactgaagagagggaatacaagtgtgatcagtgtcccaaggcatttaactggaagtccaatttaattcgccaccagatgtcacatgacagtggaaagcactatgaatgtgaaaactgtgccaaggttttcacggaccctagcaaccttcagcggcacattcgctctcagcatgtcggtgcccgggcccatgcatgcccggagtgtggcaaaacgtttgccacttcgtcgggcctcaaacaacacaagcacatccacagcagtgtgaagccctttatctgtgaggtctgccataaatcctatactcagttttcaaacctttgccgtcataagcgcatgcatgctgattgcagaacccaaatcaagtgcaaagactgtggacaaatgttcagcactacgtcttccttaaataaacacaggaggttttgtgagggcaagaaccattttgcggcaggtggattttttggccaaggcatttcacttcctggaaccccagctatggataaaacgtccatggttaatatgagtcatgccaacccgggccttgctgactattttggcgccaataggcatcctgctggtcttacctttccaacagctcctggattttcttttagcttccctggtctgtttccttccggcttgtaccacaggcctcctttgatacctgctagttctcctgttaaaggactatcaagtactgaacagacaaacaaaagtcaaagtcccctcatgacacatcctcagatactgccagctacacaggatattttgaaggcactatctaaacacccatctgtaggggacaataagccagtggagctccagcccgagaggtcctctgaagagaggccctttgagaaaatcagtgaccagtcagagagtagtgaccttgatgatgtcagtacaccaagtggcagtgacctggaaacaacctcgggctctgatctggaaagtgacattgaaagtgataaagagaaatttaaagaaaatggtaaaatgttcaaagacaaagtaagccctcttcagaatctggcttcaataaataataagaaagaatacagcaatcattccattttctcaccatctttagaggagcagactgcggtgtcaggagctgtgaatgattctataaaggctattgcttctattgctgaaaaatactttggttcaacaggactggtggggctgcaagacaaaaaagttggagctttaccttacccttccatgtttcccctcccattttttccagcattctctcaatcaatgtacccatttcctgatagagacttgagatcgttacctttgaaaatggaaccccaatcaccaggtgaagtaaagaaactgcagaagggcagctctgagtccccctttgatctcaccactaagcgaaaggatgagaagcccttgactccagtcccctccaagcctccagtgacacctgccacaagccaagaccagcccctggatctaagtatgggcagtaggagtagagccagtgggacaaagctgactgagcctcgaaaaaaccacgtgtttgggggaaaaaaaggaagcaacgtcgaatcaagacctgcttcagatggttccttgcagcatgcaagacccactcctttctttatggaccctatttacagagtagagaaaagaaaactaactgacccacttgaagctttaaaagagaaatacttgaggccttctccaggattcttgtttcacccacaaatgtcagctattgaaaacatggcagaaaagctagagagcttcagtgccctgaaacctgaggccagtgagctcttacagtcagtgccctctatgttcaacttcagggcgcctcccaatgccctgccagagaaccttctgcggaagggaaaggagcgctatacctgcagatactgtggcaagatttttccaaggtctgcaaacctaacacggcacttgagaacccacacaggagagcagccttacagatgcaaatactgtgacagatcatttagcatatcttctaacttgcaaaggcatgttcgcaacatccacaataaagagaagccatttaagtgtcacttatgtgataggtgttttggtcaacaaaccaatttagacagacacctaaagaaacatgagaatgggaacatgtccggtacagcaacatcgtcgcctcattctgaactggaaagtacaggtgcgattctggatgacaaagaagatgcttacttcacagaaattcgaaatttcattgggaacagcaaccatggcagccaatctcccaggaatgtggaggagagaatgaatggcagtcattttaaagatgaaaaggctttggtgaccagtcaaaattcagacttgctggatgatgaagaagttgaagatgaggtgttgttagatgaggaggatgaagacaatgatattactggaaaaacaggaaaggaaccagtgacaagtaatttacatgaaggaaaccctgaggatgactatgaagaaaccagtgccctggagatgagttgcaagacatccccagtgaggtataaagaggaagaatataaaagtggactttctgctctagatcatataaggcacttcacagatagcctcaaaatgaggaaaatggaagataatcaatattctgaagctgagctgtcttcttttagtacttcccatgtgccagaggaacttaagcagccgttacacagaaagtccaaatcgcaggcatatgctatgatgctgtcactgtctgacaaggagtccctccattctacatcccacagttcttccaacgtgtggcacagtatggccagggctgcggcggaatccagtgctatccagtccataagccacgtatgacgttatcaaggttgaccagagtgggaccaagtccaacagtagcatggctctttcatataggactatttacaagactgctgagcagaatgccttataaacctgcagggtcactcatctaaagtctagtgaccttaaactgaatgatttaaaaaagaaaagaaagaaaaaagaaactatttattctcgatattttgttttgcacagcaaaggcagctgctgacttctggaagatcaatcaatgcgacttaaagtgattcagtgaaaacaaaaaacttggtgggctgaaggcatcttccagtttaccccaccttagggtatgggtgggtgagaagggcagttgagatggcagcattgatatgaatgaacactccatagaaactgaattctcttttgtacaagatcacctgacatgattgggaacagttgcttttaattacagatttaatttttttcttcgttaaagttttatgtaatttaaccctttgaagacagaagtagttggatgaaatgcacagtcaattattatagaaactgataacagggagtacttgttcccccttttgccttcttaagtacattgtttaaaactagggaaaaagggtatgtgtatattgtaaactatggatgttaacactcaaagaggttaagtcagtgaagtaacctattcatcaccagtaccgctgtaccactaataaattgtttgccaaatccttgtaataacatcttaattttagacaatcatgtcactgtttttaatgtttatttttttgtgtgtgttgcgtgtatcatgtatttatttgttggcaaactattgtttgttgattaaaatagcactgttccagtcagccactactttatgacgtctgaggcacacccctttccgaatttcaaggaccaaggtgacccgacctgtgtatgagagtgccaaatggtgtttggcttttcttaacattcctttttgtttgtttgttttgttttccttcttaatgaactaaatacgaatagatgcaacttagtttttgtaatactgaaatcgattcaattgtataaacgattataatttctttcatggaagcatgattcttctgattaaaaactgtactccatattttatgctggttgtctgcaagcttgtgcgatgttatgttcatgttaatcctatttgtaaaatgaagtgttcccaaccttatgttaaaagagagaagtaaataacagactgtattcagttattttgccctttattgaggaaccagatttgttttctttttgtttgtaatctcattttgaaataatcagcaagttgaggtactttcttcaaatgctttgtacaatataaactgttatgcctttcagtgcattactatgggaggagcaactaaaaaataaagacttacaaaaagga

SEQ ID NO: 14 - DNA, 965 bp, Homo sapiens
tgtgaccagctctttgaatctaaggctgaactagcagatcaccaaaagtttccatgcagtactcctcactcagcattttcaatggttgaagaggactttcagcaaaaactcgaaagcgagaatgatctccaagagatacacacgatccaggagtgtaaggaatgtgaccaagtttttcctgatttgcaaagcctggagaaacacatgctgtcacatactgaagagagggaatacaagtgtgatcagtgtcccaaggcatttaactggaagtccaatttaattcgccaccagatgtcacatgacagtggaaagcactatgaatgtgaaaactgtgccaaggttttcacggaccctagcaaccttcagcggcacattcgctctcagcatgtcggtgcccgggcccatgcatgcccggagtgtggcaaaacgtttgccacttcgtcgggcctcaaacaacacaagcacatccacagcagtgtgaagccctttatctcattctctcaatcaatgtacccatttcctgatagagacttgagatcgttacctttgaaaatggaaccccaatcaccaggtgaagtaaagaaactgcagaagggcagctctgagtccccctttgatctcaccactaagcgaaaggatgagaagcccttgactccagtcccctccaagcctccagtgacacctgccacaagccaagaccagcccctggatctaagtatgggcagtaggagtagagccagtgggacaaagctgactgagcctcgaaaaaaccacgtgtttgggggaaaaaaaggaagcaacgtcgaatcaagacctgcttcagatggttccttgcagcatgcaagacccactcctttctttatggaccctatttacagagtagagaaaagaaaactaactgacccacttgaagctttaaaagagaaatacttgaggccttctccaggattcttgtttcacccaca

SEQ ID NO: 15 - DNA, 418 bp, Homo sapiens
tgtcccaaggcatttaactggaagtccaatttaattcgccaccagatgtcacatgacagtggaaagcactatgaatgtgaaaactgtgccaagcaggttttcacggaccctagcaaccttcagcggcacattcgctctcagcatgtcggtgcccgggcccatgcatgcccggagtgtggcaaaacgtttgccacttcgtcgggcctcaaacaacacaagcacatccacagcagtgtgaagccctttatctgtaagttttcaacactccagccctccttcccacatctgtctcttccccaaaaggcactgtgaccaggagagtggctgcacatggcctttagagggtcaccgtgttagctatttatggtccaatccagtagggctatttatgattacaagtaaatcttttataatcaga

SEQ ID NO: 16 - DNA, 2389 bp, Homo sapiens
aaagagatagaatcaaactgctttttttcgacatactggtttttctttctgtttttcttctctttcttctatttcttgtggatattatggctaataacacaacaagtttagggagtccatggccagaaaacttttgggaggaccttatcatgtccttcactgtatccatggcaatcgggctggtacttggaggatttatttgggctgtgttcatttgtctgtctcgaagaagaagagccagtgctcccatctcacagtggagttcaagcaggagatctaggtcttcttacacccacggcctcaacagaactggattttaccgccacagtggctgtgaacgtcgaagcaacctcagcctggccagtctcaccttccagcgacaagcttccctggaacaagcaaattcctttccaagaaaatcaagtttcagagcttctactttccatccctttctgcaatgtccaccacttcctgtggaaactgagagtcagctggtgactctcccttcttccaatatctctcccaccatcagcacttcccacagtctgagccgtcctgactactggtccagtaacagtcttcgagtgggcctttcaacaccgcccccacctgcctatgagtccatcatcaaggcattcccagattcctgaggtcttgatccgtcgcccaggcgggagttgcagtagcatgatcaggactcactgcagcctctatctcccaggctcaagtaatcctcccatcttagtgccccaagtagctgggactacaggggtgcactaccacaccgggttgaatttttttttaattttagtagagatgaagtgtcactatgttaccaaggctggtctcaaactcctaaactcagatgatcctcctgcctcggcctcccaaagtgctggaattagcctggccaatcttggatttttaatggaatatgtgggcacaaaatgacagaacataggacattctaaagttccttgatttgatcattataagaagtgtgggactcaagcacaggaaactgaactcttttggtgtcattggatgtttcatttttgacactaattttttctggacaaactctttatgtgtttttcccaagaatagttatctacttcctggaggcaaaatccttggatttactaacatgatgatttaccttttcttcaccgttgtcgttacattgttagaaaagcaacaggaaaaaatccaattcatttgacctaaaaacaagcctcaagtttaaaaccaagctcacgtttttcttaagggaaaaattttctttcttaaacttacatctagcaacttggaaagcactttctctggggatcttcttttgtaactttgcagacaaataagtatgagtcactggggagagagtttgttattgaaatagatgttgcccatgaagaattctccttcctggattgactcttaatcatcaggcatcattcctggtttgcttctctacgaatctcaattccaacttctctgcagagtctgtacagtgattaagccatgccagatggtctttggtgcacacagttatttaagaatccacttccacaggtggctgcccttgtaaggaagaatgcatccctaaatgtggccaccagagagttccagtgggcagatgtctgtggctgcccttctcatttaaggacatgagttcactggagtattactcaaaaagtctgtggttcatttccagtattgtgaatatttagtttatgtggccgtttctttgtttctttgaacagtgggattttcagtgaaaaagtaccctctttttcatttcctattgcagtggtcacagctaatagtgtctgaacatggttcaagaataagagattccatgtagcattttctttattattttcatttcccttatattatccatcattccttaaggacaattattcttaataatgcttatagaaaatgttctctaattaaacatgccaaaaggaaaaagtaagagaaagagggagcaagaagaaaatggaagaaaaagggaaaaaagctaaccggataaccaatttgttataagttggttttcaacaaagaaatttagcagccaagtaaggtttcaagggaatattaacttggtatcagggctacttttttttttttttttttacttgcatgtcatccttaatgtctaacatgaaaaatcagcaaagagtatggtttttatcaagaatttgtgttgggagtaaaaactgctttatagctcccaaattaggaagagaagagcagaaatcctctggggcatttaaccatctggcagaattgttgctgcacccttatcccagttataagacagtcaaaatgactatttcctaaatattgtgagtgtatgaaatgtgaaattaaagcaaaaactgga

SEQ ID NO: 17 - DNA, 10032 bp, Homo sapiens
atctccgccgccacctcggtctcgctgctcctgggcggggggtgaggacgagtccggagtatctggggtttggcgttgttgtcagcctcggggagagagattggacaaatattctccaagaggaggagggcgacgccaaggactttccacatcaactgctttggggtatctccacaagttggaagagggaccctttcgttttgcattgcgtgtgttgtgctcattaccagtgcagcgactgccgtcccagggtgactctgagttgtcctttatcgtgagctagcaatggctagcgaagacaatcgtgtcccttccccgccaccaacaggtgatgacgggggaggtggagggagagaagaaacccctactgaagggggtgcattgtctctgaaaccagggctccccatcaggggcatcagaatgaaatttgccgtgttgaccggtttggttgaagttggagaagtatccaatagggatattgtagaaactgtctttaacctgttggtaggaggacagtttgatctggaaatgaatttcattatccaagaaggtgagagtattaactgcatggtggacctactggaaaaatgtgacattacgtgccaagcagaagtctggagcatgtttacagccattctgaagaaaagcatacggaatcttcaagtctgcactgaagtaggccttgttgaaaaagtgcttgggaaaattgaaaaagttgacaatatgatagcagatcttttggttgacatgttgggagtgctggctagctataatttgacagttcgcgagctaaagcttttcttcagtaaacttcaaggagataaaggacgatggcctccacatgctgggaagttgctgtctgtgttaaagcatatgcctcagaagtatggtcctgatgccttttttaactttccaggaaagagtgctgcagctattgcattacctcctatagccaaatggccataccagaatggttttacatttcatacatggcttagaatggatcctgtaaataacatcaatgtagataaggataaaccatatttgtattgtttcagaaccagcaaaggtcttggctattctgctcattttgttggaggctgtttgattgtaacatcaataaagtcaaaaggaaaaggctttcaacactgtgtgaaatttgatttcaagccacaaaagtggtatatggttaccatagtacacatctataaccgatggaagaatagtgaacttcgatgttatgtgaatggtgagctggcttcctatggagagataacatggtttgtcaacactagcgatacctttgacaaatgtttcctgggctcatcagaaacagcagatgctaatagagtattctgtggtcagatgactgcagtttaccttttcagtgaagctctaaatgcagctcagatatttgctatttatcagttgggcctgggatacaagggtacatttaaattcaaagcagaaagcgaccttttccttgctgagcatcacaaacttttattgtacgatgggaaactctctagtgccattgcattcacgtacaatccacgggctacagatgcccagctttgtcttgaatcatctcctaaggacaacccttcaatttttgttcattcaccacatgcactcatgctccaggatgtaaaggcagttttaacacattccatccaaagtgcaatgcattcaattggaggagtacaagtactatttccactttttgcacagttggattacaggcaatatttgtctgatgagattgatttgactatatgttcaaccttgctggcctttatcatggaattgttgaagaactcaattgctatgcaggaacagatgcttgcctgtaagggcttcttggtaataggatatagccttgaaaagtcttccaaatctcatgttagcagagcagtacttgaactttgccttgcattttcaaaatatctgagtaatctgcagaatgggatgcccctgctcaagcaattgtgtgatcacgttcttcttaatcctgccatatggattcataccccagccaaggttcaactgatgctctatacttatctgtccacggaattcattggtacagtcaacatatataacaccattcggagagttggaacagtgcttctcatcatgcacacgctgaagtactactactgggcagtgaatcctcaggatcgaagtggtatcaccccaaaaggattagatggaccgcgacctaatcaaaaagaaatgctttctctacgagcattcttgttgatgttcattaagcaattagtgatgaaggattctggagtaaaggaagatgaattacaggccattcttaattacctactgactatgcatgaggatgacaatctaatggatgtcctacagctgcttgttgcattaatgtcagaacaccctaactctatgattcctgcttttgaccaaaggaatgggttacgtgttatctacaaacttctggcatcgaaaagtgaaggaatcagggtacaagctcttaaggcaatgggttattttttaaaacatctggccccaaagaggaaagcagaagtcatgcttggacatggattgttttcattgctagctgaaaggctcatgcttcagacaaatttaatcacaatgaccacatataatgtgctgtttgagattcttatagaacagattggtactcaggtgatacataaacagcatccagatcctgattcttcagtgaagatacaaaaccctcagatactaaaagtaattgcgaccctacttcgaaattctccccagtgcccagagagcatggaggttcgcagagcctttctttctgacatgattaaactttttaataacagtagagaaaacaggaggagcttgctacaatgctctgtgtggcaagaatggatgctttctctctgctattttaatcctaagaattcagatgagcaaaagataacagaaatggtatacgccatattcagaatcctgctttaccatgcagtcaaatatgagtggggtggctggcgtgtatgggtagacactttatcaatcactcattcaaaggtcacttttgaaatacacaaagaaaaccttgccaatatatttagggaacagcaaggaaaagttgatgaagaaatagggctgtgttcttcaacttcagttcaagcagcctctggcattagaagggatattaatgtttcagtaggatcccagcaaccagatacgaaggattctcctgtctgtcctcatttcaccacaaatggtaatgaaaattcaagtatagagaagacaagttcactagaatctgcatctaatattgaactgcaaactactaatacatcttatgaagaaatgaaagctgagcaagaaaatcaggagttaccagatgaaggcactttggaagaaacactgacaaatgagacaaggaatgcagatgatttagaagtatcttctgacataatagaagctgtggctatttcctctaattcttttataacaactggcaaagattcaatgactgtcagtgaagtaactgcttctataagttctccttcagaagaggatgcctcagagatgccagaattcttggataaatctatagtagaggaagaggaagatgatgattatgtggaactgaaagtagaaggcagtcctactgaggaagctaatctacccacagagctccaagataacagtttgtctccagctgcatctgaagccggtgaaaaactggacatgtttggtaatgatgacaaattaatatttcaagaaggaaaacctgttactgaaaagcaaactgatactgaaactcaagattctaaagattctggaattcagactatgacagcatcagggtcttcagctatgtcaccagaaactactgtttcccaaatagctgtagaatcagaccttggtcagatgctggaggaagggaagaaagcaactaacctcactagagaaaccaaattaattaatgattgtcatggtagtgtctctgaggcttcttctgagcaaaagattgcgaagttggatgtttccaatgttgctacagatactgagaggctggagttgaaggccagtcccaacgtggaagcacctcaacctcatcgacatgtgcttgagatatcaaggcaacatgagcagccagggcaaggaatagcaccagatgcagttaatggacaaaggagggattccagatctactgtgtttcgtattcctgagttcaactggtctcagatgcatcaacgtttgctcactgatctattattttcaatagaaacagatatacagatgtggagaagccattcaacaaagacagttatggacttcgtgaatagcagtgataatgtcatctttgtacacaacacaattcatctcatctctcaagtgatggacaatatggtcatggcttgtgggggtatactgccattgctttcagctgctacatcggctacacatgaactggaaaatattgaacctactcaaggcctttcaatagaagcctctgtgacatttttgcagaggctaattagccttgtggatgtgcttatatttgcaagttctcttggctttactgaaattgaagctgaaaaaagtatgtcatctggaggaattttgcggcagtgtctccgactagtttgtgcagtcgcagtaaggaattgcttggagtgtcaacagcattcacaactgaaaactaggggagataaagccttgaaaccaatgcatagccttattcctttagggaaatctgcagcgaagagcccagtggacattgtgactggcggtatatctccagtaagagatcttgacaggcttctacaggacatggatattaatcggcttagggcagttgttttcagagacatagaggatagcaaacaagctcaatttttagccttggcagtagtatactttatctctgttcttatggtctccaagtacagagacattttggaaccccaaaatgaaaggcatagccagtcatgtacagaaactggcagtgaaaatgagaatgtatcactctctgaaatcacaccagcagcattcagcactttaactacggcatcagtggaagaatctgaaagcacatcatctgctcgaaggagggactcaggcattggggaagaaacagccactggtttaggaagccatgtggaagtaactcctcacacagcacctcctggtgtcagtgcaggcccagatgcaatcagcgaggtgctatctactctttctttagaagtcaataagtctccggaaaccaaaaatgatagaggaaatgacttggacactaaggctacaccgtcagtttcagtttcaaaaaacgtcaatgtgaaagacattctccgaagcttggttaacataccagcagatggagtcacagtggatcctgcccttctgccaccagcctgccttggagcccttggtgatctatctgtggaacaacccgtgcagttcagatcttttgacagaagtgtcattgttgcagcaaaaaagtcagcagtctcaccttccacctttaatacaagcatacctaccaatgctgtcagtgtggtttcctcagtagattcagcccaagcctcagatatgggaggagaatcaccaggcagtagatcatctaatgcaaaattgccctcagttccaacagttgattcagtttcacaagatccggtttcaaatatgagtattacagagaggcttgaacacgctttggaaaaggcagctcctctccttcgtgagatttttgtggattttgcaccttttctttctcggacacttttgggtagccatggacaagaactgcttatagaaggaacaagtctggtttgcatgaagtcgagtagttcagttgtggaattggttatgctactgtgttctcaggagtggcaaaattctattcagaagaatgcaggccttgcttttatcgaacttgtcaatgaaggaaggttgcttagccagacaatgaaggatcatctagtaagagtagcaaatgaagctgaatttatcctgagcaggcagagagcagaagatattcacagacatgcggaatttgagtcactgtgtgcccagtattctgcagacaaacgagaagatgagaagatgtgtgatcatttgataagagcagcaaaatatcgtgaccacgtgacagcaactcaactaatccagaaaattatcaacattctcacagacaagcatggagcctggggaaattctgcagtgagtcgtcctcttgagttctggcgccttgactactgggaagatgacttgcggcgccggcgacgatttgtgcgtaaccctctaggatcgacacatcctgaagcgacactaaaaacagccgtggaacatgccacagatgaagatatccttgctaaaggaaaacagtccatcaggagtcaggctttaggaaatcagaactcagaaaacgagatcctcctggaaggcgatgatgatactctgtcatccgtggatgagaaagatttagagaatcttgccggtcctgttagcctgagcacaccagctcagcttgtggccccctctgttgtagtaaagggcactctttctgtcacctcctccgaactctattttgaggtggatgaagaggatcctaacttcaaaaaaatcgaccccaagatcttggcatatacagaagggctgcatggaaaatggctgttcacagagatacgatcaatcttttctcgtcgttatcttttgcaaaatacagccctggagatctttatggcaaacagagttgctgtgatgttcaacttcccagaccctgcaacagtaaagaaagtggttaactatctacctcgtgttggcgttggaacaagttttggattgcctcaaaccagacgtatttcattagctagtccacgtcagctttttaaggcttctaatatgacccagcgatggcaacacagagagatatctaattttgagtacttgatgtttctcaacacgatagcaggacggagttataatgacttaaatcagtatccagtgtttccttgggtcatcactaattatgaatcagaagaactggatcttaccttgcccaccaacttcagagatttgtccaagccaataggagctctgaacccaaaaagagcagcattcttcgctgagcgttatgaatcatgggaagatgatcaagttccaaagtttcactatggtactcattactcaactgcaagttttgttcttgcatggctgctaagaatagaaccctttacaacttatttcctaaatttgcaaggaggcaaatttgatcatgcagatcgaactttttcatcaatttccagagcttggcgaaacagtcagcgtgatacctctgatattaaggagttgatccctgaattttattatctccctgagatgtttgtcaacttcaataattataatcttggagtgatggatgatgggacagtagtgtctgatgtcgaacttcctccttgggccaaaacctcagaagaatttgttcacataaacagattggccctggagagtgaatttgtttcctgccagcttcaccaatggattgatctcatttttggctataaacagcaaggaccagaagctgtccgagccctcaatgtgttctattacttgacctatgaaggagctgtcaatctgaattcaataactgatcctgtgttgagagaggctgttgaagctcaaatccgaagttttggacagactccttctcaactactcatagagccccatcctcccagaggttctgccatgcaagtgagtccattgatgttcacagacaaagcccagcaggatgttatcatggtcctcaagtttccctccaactcccctgttactcacgtggcagccaacacccagcctggtttggcaactcccgctgtgatcacagtcactgctaacaggttatttgcggtgaacaaatggcacaaccttcctgctcatcaaggtgctgtacaagaccagccataccagctgccagtggaaatcgatcctctcatagccagcaatacaggaatgcacaggaggcaaatcactgaccttttagaccaaagtattcaagtgcattcccagtgctttgtcatcacttcagacaaccgctatattctcgtctgtggcttctgggataaaagtttcagagtctattctacagacacaggaagattgatccaagtggtgtttggccattgggatgtcgtcacttgccttgctcgttctgagtcatatattgggggaaattgctacattctctcagggtcacgtgatgcaactcttttgctgtggtattggaatggaaaatgcagtgggattggagataacccaggtgagactgctgctcctcgggccattttgaccggccatgactatgaggtcacatgtgctgcggtgtgtgcggagctaggcctggtgttgagtggttcacaagaaggaccatgtctcatacattccatgaatggagacttgttgaggaccttggagggtcctgaaaactgcctgaaaccaaaactcattcaggcttcaagagagggtcattgtgtcatattctatgaaaacggcctcttctgtacattcagtgtgaatggaaaactccaggccacgatggaaacagatgataacataagagccatccagctgagccgagatgggcagtacctgctcacaggaggagacagaggagtggtcgtggtccggcaggtgtcggacctcaagcagctctttgcctatccaggatgtgacgctggaatccgggccatggcgctgtcttacgaccagaggtgcatcatttctggcatggcttcaggaagcattgtgctattttacaacgactttaaccggtggcatcatgaataccaaacccgctactgatggtgacagctgtacatcaactctgcccctaggatgagcagaagtacctggagcatcattctcttctaccacatctgaatgtaacttaaatttgctcaaagaagcaaaatattttttaaagttattaataattgctatttttgtagtctttgcttgacttttttgggggggattagcaaagcagaaaactggctgctgggttctgtagttttaaaaaatctatatttttagtcataatgagaagtctattttcaccaattatggaaacatacagtggagcaagtaaaccacttattccagctataatattatgaagaacatttcccatgcatctacaagcttgagaaataggattttcacagtggggaagtgggttacaatattggaataggaaaatttgatgctgtaatttgggtcctgtatttttccaaacaactgtgcttcttcagcactaatatttctgggatttaaatagtaatgtttaaattatggtaaatgtaatttaaaacatctcaattaagtctgtctatcaatattgttcttcaagcatgttttagacctagaaaagtatggtttgggggaggctattttattgtgtggttagcacaaggaatctaatttatagcaagtgtgaaatagttaccattttttcctgatctgtcatcttcatagcacaacaaaacgaaatgatggaaatgctcttgagctcacaacatttgtttttcttttaaagtaaatgcaagtaccaaagctcactactgcggtttgcctgtgcctggacaatgaggcggagccactgttgttttgttggggcacccccttccctccccgggtttgcaaatagaggctaccgggtgctgtattcagcaacacctgttttactatttgttattaaactatcatctccaccttccttttgattagcaatttgtactaagaaacgaaacaatgttatttggtggtgtataattctacttttctagtagattactgtgtggaattctgtgaaaaatatttgagaaaaggcctgtattgcataaataaattctttgtatgttgtgaactttgatctctatttctcttgcaacagacttcaagtggtccctctttttttcctacacactcagctcttctctaaaagcaaggatttggaatgaacaattcagtttgacagaaaaagctgaataaaat

SEQ ID NO: 18 - DNA, 9899 bp, Homo sapiens
agggaaacttggaggctgcgaccagggtttggcgttgttgtcagcctcggggagagagattggacaaatattctccaagaggaggagggcgacgccaaggactttccacatcaactgctttggggtatctccacaagttggaagagggaccctttcgttttgcattgcgtgtgttgtgctcattaccagtgcagcgactgccgtcccagggtgactctgagttgtcctttatcgtgagctagcaatggctagcgaagacaatcgtgtcccttccccgccaccaacaggtgatgacgggggaggtggagggagagaagaaacccctactgaagggggtgcattgtctctgaaaccagggctccccatcaggggcatcagaatgaaatttgccgtgttgaccggtttggttgaagttggagaagtatccaatagggatattgtagaaactgtctttaacctgttggtaggaggacagtttgatctggaaatgaatttcattatccaagaaggtgagagtattaactgcatggtggacctactggaaaaatgtgacattacgtgccaagcagaagtctggagcatgtttacagccattctgaagaaaagcatacggaatcttcaagtctgcactgaagtaggccttgttgaaaaagtgcttgggaaaattgaaaaagttgacaatatgatagcagatcttttggttgacatgttgggagtgctggctagctataatttgacagttcgcgagctaaagcttttcttcagtaaacttcaaggagataaaggacgatggcctccacatgctgggaagttgctgtctgtgttaaagcatatgcctcagaagtatggtcctgatgccttttttaactttccaggaaagagtgctgcagctattgcattacctcctatagccaaatggccataccagaatggttttacatttcatacatggcttagaatggatcctgtaaataacatcaatgtagataaggataaaccatatttgtattgtttcagaaccagcaaaggtcttggctattctgctcattttgttggaggctgtttgattgtaacatcaataaagtcaaaaggaaaaggctttcaacactgtgtgaaatttgatttcaagccacaaaagtggtatatggttaccatagtacacatctataaccgatggaagaatagtgaacttcgatgttatgtgaatggtgagctggcttcctatggagagataacatggtttgtcaacactagcgatacctttgacaaatgtttcctgggctcatcagaaacagcagatgctaatagagtattctgtggtcagatgactgcagtttaccttttcagtgaagctctaaatgcagctcagatatttgctatttatcagttgggcctgggatacaagggtacatttaaattcaaagcagaaagcgaccttttccttgctgagcatcacaaacttttattgtacgatgggaaactctctagtgccattgcattcacgtacaatccacgggctacagatgcccagctttgtcttgaatcatctcctaaggacaacccttcaatttttgttcattcaccacatgcactcatgctccaggatgtaaaggcagttttaacacattccatccaaagtgcaatgcattcaattggaggagtacaagtactatttccactttttgcacagttggattacaggcaatatttgtctgatgagattgatttgactatatgttcaaccttgctggcctttatcatggaattgttgaagaactcaattgctatgcaggaacagatgcttgcctgtaagggcttcttggtaataggatatagccttgaaaagtcttccaaatctcatgttagcagagcagtacttgaactttgccttgcattttcaaaatatctgagtaatctgcagaatgggatgcccctgctcaagcaattgtgtgatcacgttcttcttaatcctgccatatggattcataccccagccaaggttcaactgatgctctatacttatctgtccacggaattcattggtacagtcaacatatataacaccattcggagagttggaacagtgcttctcatcatgcacacgctgaagtactactactgggcagtgaatcctcaggatcgaagtggtatcaccccaaaaggattagatggaccgcgacctaatcaaaaagaaatgctttctctacgagcattcttgttgatgttcattaagcaattagtgatgaaggattctggagtaaaggaagatgaattacaggccattcttaattacctactgactatgcatgaggatgacaatctaatggatgtcctacagctgcttgttgcattaatgtcagaacaccctaactctatgattcctgcttttgaccaaaggaatgggttacgtgttatctacaaacttctggcatcgaaaagtgaaggaatcagggtacaagctcttaaggcaatgggttattttttaaaacatctggccccaaagaggaaagcagaagtcatgcttggacatggattgttttcattgctagctgaaaggctcatgcttcagacaaatttaatcacaatgaccacatataatgtgctgtttgagattcttatagaacagattggtactcaggtgatacataaacagcatccagatcctgattcttcagtgaagatacaaaaccctcagatactaaaagtaattgcgaccctacttcgaaattctccccagtgcccagagagcatggaggttcgcagagcctttctttctgacatgattaaactttttaataacagtagagaaaacaggaggagcttgctacaatgctctgtgtggcaagaatggatgctttctctctgctattttaatcctaagaattcagatgagcaaaagataacagaaatggtatacgccatattcagaatcctgctttaccatgcagtcaaatatgagtggggtggctggcgtgtatgggtagacactttatcaatcactcattcaaaggtcacttttgaaatacacaaagaaaaccttgccaatatatttagggaacagcaaggaaaagttgatgaagaaatagggctgtgttcttcaacttcagttcaagcagcctctggcattagaagggatattaatgtttcagtaggatcccagcaaccagatacgaaggattctcctgtctgtcctcatttcaccacaaatggtaatgaaaattcaagtatagagaagacaagttcactagaatctgcatctaatattgaactgcaaactactaatacatcttatgaagaaatgaaagctgagcaagaaaatcaggagttaccagatgaaggcactttggaagaaacactgacaaatgagacaaggaatgcagatgatttagaagtatcttctgacataatagaagctgtggctatttcctctaattcttttataacaactggcaaagattcaatgactgtcagtgaagtaactgcttctataagttctccttcagaagaggatgcctcagagatgccagaattcttggataaatctatagtagaggaagaggaagatgatgattatgtggaactgaaagtagaaggcagtcctactgaggaagctaatctacccacagagctccaagataacagtttgtctccagctgcatctgaagccggtgaaaaactggacatgtttggtaatgatgacaaattaatatttcaagaaggaaaacctgttactgaaaagcaaactgatactgaaactcaagattctaaagattctggaattcagactatgacagcatcagggtcttcagctatgtcaccagaaactactgtttcccaaatagctgtagaatcagaccttggtcagatgctggaggaagggaagaaagcaactaacctcactagagaaaccaaattaattaatgattgtcatggtagtgtctctgaggcttcttctgagcaaaagattgcgaagttggatgtttccaatgttgctacagatactgagaggctggagttgaaggccagtcccaacgtggaagcacctcaacctcatcgacatgtgcttgagatatcaaggcaacatgagcagccagggcaaggaatagcaccagatgcagttaatggacaaaggagggattccagatctactgtgtttcgtattcctgagttcaactggtctcagatgcatcaacgtttgctcactgatctattattttcaatagaaacagatatacagatgtggagaagccattcaacaaagacagttatggacttcgtgaatagcagtgataatgtcatctttgtacacaacacaattcatctcatctctcaagtgatggacaatatggtcatggcttgtgggggtatactgccattgctttcagctgctacatcggctacacatgaactggaaaatattgaacctactcaaggcctttcaatagaagcctctgtgacatttttgcagaggctaattagccttgtggatgtgcttatatttgcaagttctcttggctttactgaaattgaagctgaaaaaagtatgtcatctggaggaattttgcggcagtgtctccgactagtttgtgcagtcgcagtaaggaattgcttggagtgtcaacagcattcacaactgaaaactaggggagataaagccttgaaaccaatgcatagccttattcctttagggaaatctgcagcgaagagcccagtggacattgtgactggcggtatatctccagtaagagatcttgacaggcttctacaggacatggatattaatcggcttagggcagttgttttcagagacatagaggatagcaaacaagctcaatttttagccttggcagtagtatactttatctctgttcttatggtctccaagtacagagacattttggaaccccaaaatgaaaggcatagccagtcatgtacagaaactggcagtgaaaatgagaatgtatcactctctgaaatcacaccagcagcattcagcactttaactacggcatcagtggaagaatctgaaagcacatcatctgctcgaaggagggactcaggcattggggaagaaacagccactggtttaggaagccatgtggaagtaactcctcacacagcacctcctggtgtcagtgcaggcccagatgcaatcagcgaggtgctatctactctttctttagaagtcaataagtctccggaaaccaaaaatgatagaggaaatgacttggacactaaggctacaccgtcagtttcagtttcaaaaaacgtcaatgtgaaagacattctccgaagcttggttaacataccagcagatggagtcacagtggatcctgcccttctgccaccagcctgccttggagcccttggtgatctatctgtggaacaacccgtgcagttcagatcttttgacagaagtgtcattgttgcagcaaaaaagtcagcagtctcaccttccacctttaatacaagcatacctaccaatgctgtcagtgtggtttcctcagtagattcagcccaagcctcagatatgggaggagaatcaccaggcagtagatcatctaatgcaaaattgccctcagttccaacagttgattcagtttcacaagatccggtttcaaatatgagtattacagagaggcttgaacacgctttggaaaaggcagctcctctccttcgtgagatttttgtggattttgcaccttttctttctcggacacttttgggtagccatggacaagaactgcttatagaaggaacaagtctggtttgcatgaagtcgagtagttcagttgtggaattggttatgctactgtgttctcaggagtggcaaaattctattcagaagaatgcaggccttgcttttatcgaacttgtcaatgaaggaaggttgcttagccagacaatgaaggatcatctagtaagagtagcaaatgaagctgaatttatcctgagcaggcagagagcagaagatattcacagacatgcggaatttgagtcactgtgtgcccagtattctgcagacaaacgagaagatgagaagatgtgtgatcatttgataagagcagcaaaatatcgtgaccacgtgacagcaactcaactaatccagaaaattatcaacattctcacagacaagcatggagcctggggaaattctgcagtgagtcgtcctcttgagttctggcgccttgactactgggaagatgacttgcggcgccggcgacgatttgtgcgtaaccctctaggatcgacacatcctgaagcgacactaaaaacagccgtggaacatgtgtgcatttttaaattgagagagaacagcaaagccacagatgaagatatccttgctaaaggaaaacagtccatcaggagtcaggctttaggaaatcagaactcagaaaacgagatcctcctggaaggcgatgatgatactctgtcatccgtggatgagaaagatttagagaatcttgccggtcctgttagcctgagcacaccagctcagcttgtggccccctctgttgtagtaaagggcactctttctgtcacctcctccgaactctattttgaggtggatgaagaggatcctaacttcaaaaaaatcgaccccaagatcttggcatatacagaagggctgcatggaaaatggctgttcacagagatacgatcaatcttttctcgtcgttatcttttgcaaaatacagccctggagatctttatggcaaacagagttgctgtgatgttcaacttcccagaccctgcaacagtaaagaaagtggttaactatctacctcgtgttggcgttggaacaagttttggattgcctcaaaccagacgtatttcattagctagtccacgtcagctttttaaggcttctaatatgacccagcgatggcaacacagagagatatctaattttgagtacttgatgtttctcaacacgatagcaggacggagttataatgacttaaatcagtatccagtgtttccttgggtcatcactaattatgaatcagaagaactggatcttaccttgcccaccaacttcagagatttgtccaagccaataggagctctgaacccaaaaagagcagcattcttcgctgagcgttatgaatcatgggaagatgatcaagttccaaagtttcactatggtactcattactcaactgcaagttttgttcttgcatggctgctaagaatagaaccctttacaacttatttcctaaatttgcaaggaggcaaatttgatcatgcagatcgaactttttcatcaatttccagagcttggcgaaacagtcagcgtgatacctctgatattaaggagttgatccctgaattttattatctccctgagatgtttgtcaacttcaataattataatcttggagtgatggatgatgggacagtagtgtctgatgtcgaacttcctccttgggccaaaacctcagaagaatttgttcacataaacagattggccctggagagtgaatttgtttcctgccagcttcaccaatggattgatctcatttttggctataaacagcaaggaccagaagctgtccgagccctcaatgtgttctattacttgacctatgaaggagctgtcaatctgaattcaataactgatcctgtgttgagagaggctgttgaagctcaaatccgaagttttggacagactccttctcaactactcatagagccccatcctcccagaggttctgccatgcaagtgagtccattgatgttcacagacaaagcccagcaggatgttatcatggtcctcaagtttccctccaactcccctgttactcacgtggcagccaacacccagcctggtttggcaactcccgctgtgatcacagtcactgctaacaggttatttgcggtgaacaaatggcacaaccttcctgctcatcaaggtgctgtacaagaccagccataccagctgccagtggaaatcgatcctctcatagccagcaatacaggaatgcacaggaggcaaatcactgaccttttagaccaaagtattcaagtgcattcccagtgctttgtcatcacttcagacaaccgctatattctcgtctgtggcttctgggataaaagtttcagagtctattctacagacacaggaagattgatccaagtggtgtttggccattgggatgtcgtcacttgccttgctcgttctgagtcatatattgggggaaattgctacattctctcagggtcacgtgatgcaactcttttgctgtggtattggaatggaaaatgcagtgggattggagataacccaggcagtgagactgctgctcctcgggccattttgaccggccatgactatgaggtcacatgtgctgcggtgtgtgcggagctaggcctggtgttgagtggttcacaagaaggaccatgtctcatacattccatgaatggagacttgttgaggaccttggagggtcctgaaaactgcctgaaaccaaaactcattcaggcttcaagagagggtcattgtgtcatattctatgaaaacggcctcttctgtacattcagtgtgaatggaaaactccaggccacgatggaaacagatgataacataagagccatccagctgagccgagatgggcagtacctgctcacaggaggagacagaggagtggtcgtggtccggcaggtgtcggacctcaagcagctctttgcctatccaggatgtgacgctggaatccgggccatggcgctgtcttacgaccagaggtgcatcatttctggcatggcttcaggaagcattgtgctattttacaacgactttaaccggtggcatcatgaataccaaacccgctactgatggtgacagctgtacatcaactctgcccctaggatgagcagaagtacctggagcatcattctcttctaccacatctgaatgtaacttaaatttgctcaaagaagcaaaatattttttaaagttattaataattgctatttttgtagtctttgcttgacttttttgggggggattagcaaagcagaaaactggctgctgggttctgtagttttaaaaaatctatatttttagtcataatgagaagtctattttcaccaattatggaaacatacagtggagcaagtaaaccacttattccagctataatattatgaagaacatttcccatgcatctacaagcttgagaaataggattttcacagtggggaagtgggttacaatattggaataggaaaatttgatgctgtaatttgggtcctgtatttttccaaacaactgtgcttcttcagcactaatatttctgggatttaaatagtaatgtttaaattatggtaaatgtaatttaaaacatctcaattaagtctgtctatcaatattgttcttcaagcatgttttagacctagaaaagtatggtttgggggaggctattttattgtgtggttagcacaaggaatctaatttatagcaagtgtgaaatagttaccattttttcctgatctgtcatcttcatagcacaacaaaacgaaatgatggaaatgctcttgagctcacaacatttgtttttcttttaaagtaaatgcaagtaccaaagctcactactgcggtttgcctgtgcctggacaatgaggcggagccactgttgttttgttggggcacccccttccctccccgggtttgcaaatagaggctaccgggtgctgtattcagcaacacctgttttactatttgttattaaactatcatctccaccttccttttgattagcaatttgtactaagaaacgaaacaatgttatttggtggtgtataattctacttttctagtagattactgtgtggaattctgtgaaaaatatttgagaaaaggcctgtattgcataaataaattctttgtatgttgtgaa

SEQ ID NO: 19 - DNA, 4900 bp, Homo sapiens; identical to SEQ ID NO: 11
  CDS (140)..(3490)

SEQ ID NO: 20 - protein, 1116 aa, Homo sapiens
MILDEFYNVKFCIDASQPDVGSWLKYIRFAGCYDQHNLVACQINDQIFYRVVADIAPGEELLLFMKSEDYPHETMAPDIHEERQYRCEDCDQLFESKAELADHQKFPCSTPHSAFSMVEEDFQQKLESENDLQEIHTIQECKECDQVFPDLQSLEKHMLSHTEEREYKCDQCPKAFNWKSNLIRHQMSHDSGKHYECENCAKQVFTDPSNLQRHIRSQHVGARAHACPECGKTFATSSGLKQHKHIHSSVKPFICEVCHKSYTQFSNLCRHKRMHADCRTQIKCKDCGQMFSTTSSLNKHRRFCEGKNHFAAGGFFGQGISLPGTPAMDKTSMVNMSHANPGLADYFGANRHPAGLTFPTAPGFSFSFPGLFPSGLYHRPPLIPASSPVKGLSSTEQTNKSQSPLMTHPQILPATQDILKALSKHPSVGDNKPVELQPERSSEERPFEKISDQSESSDLDDVSTPSGSDLETTSGSDLESDIESDKEKFKENGKMFKDKVSPLQNLASINNKKEYSNHSIFSPSLEEQTAVSGAVNDSIKAIASIAEKYFGSTGLVGLQDKKVGALPYPSMFPLPFFPAFSQSMYPFPDRDLRSLPLKMEPQSPGEVKKLQKGSSESPFDLTTKRKDEKPLTPVPSKPPVTPATSQDQPLDLSMGSRSRASGTKLTEPRKNHVFGGKKGSNVESRPASDGSLQHARPTPFFMDPIYRVEKRKLTDPLEALKEKYLRPSPGFLFHPQFQLPDQRTWMSAIENMAEKLESFSALKPEASELLQSVPSMFNFRAPPNALPENLLRKGKERYTCRYCGKIFPRSANLTRHLRTHTGEQPYRCKYCDRSFSISSNLQRHVRNIHNKEKPFKCHLCDRCFGQQTNLDRHLKKHENGNMSGTATSSPHSELESTGAILDDKEDAYFTEIRNFIGNSNHGSQSPRNVEERMNGSHFKDEKALVTSQNSDLLDDEEVEDEVLLDEEDEDNDITGKTGKEPVTSNLHEGNPEDDYEETSALEMSCKTSPVRYKEEEYKSGLSALDHIRHFTDSLKMRKMEDNQYSEAELSSFSTSHVPEELKQPLHRKSKSQAYAMMLSLSDKESLHSTSHSSSNVWHSMARAAAESSAIQSISHV

SEQ ID NO: 21 - DNA, 5740 bp, Homo sapiens
  CDS (1202)..(4330)
cacacacacacacacacaccacacttgtgctttcaagacatcgaaacggaggctatttccctggggaaagaaatcctgcctggcgagatctccccattggttgtttacccggagaaatctacatgtttaagggggatggtgcatccataatcagtctgtccctataggacttgggtcttggcgacctttttgtgacctctcccgccagaggaggctgctgtcactttaaaaatttaaaagaggagcccgtctggcttccgatcagatcactctgggcggcgggagatagctccctttctccctcgccccgggttctttctggatggccgagcagatcctctttaaagagacagttcatgaaatagaaacccggcggctgagcttggagttgcgaaaggggacgatcccgtgagggtccaggacccgcgaaggcgctgcggaggatctgaaagggggatagagctcccctcgcctccccaggccccccaccttttcaaactctcctcctcctgcttgttttcccccattggaactgggaaggagaagtagaagttttagtgggtttcagataactttcattacacatcgggctgataagagcaagagaaagtgagaaaagagggaggtgatgtgaaccagaaggaatagctccgagctcatttaggaagggggaaaaagccaaaacacaccaaacccgggtcacccagacgaaagaagacttcatttcttgtattaaaaatacactgttggcggacaataaatccgaaacgcgtggtcctggagagcagatcctagagacggacaaagttgtcagagacccatttggaaatcgagacgcgaggcttttaaaaaattattattattatttttaaacatctctaaatgttgctcgggatcgtttgaaaggattttcgtgcaggagcgttgggggctgctgattattttattttgtttattttgattcttctgtgaatgcctattattgctgagttgaggccatagaaatctaaagatcttagacgaattttacaatgtgaagttctgcatagatgccagtcaaccagatgttggaagctggctcaagtacattagattcgctggctgttatgatcagcacaaccttgttgcatgccagataaatgatcagatattctatagagtagttgcagacattgcgccgggagaggagcttctgctgttcatgaagagcgaagactatccccatgaaactatggcgccggatatccacgaagaacggcaatatcgctgcgaagactgtgaccagctctttgaatctaaggctgaactagcagatcaccaaaagtttccatgcagtactcctcactcagcattttcaatggttgaagaggactttcagcaaaaactcgaaagcgagaatgatctccaagagatacacacgatccaggagtgtaaggaatgtgaccaagtttttcctgatttgcaaagcctggagaaacacatgctgtcacatactgaagagagggaatacaagtgtgatcagtgtcccaaggcatttaactggaagtccaatttaattcgccaccagatgtcacatgacagtggaaagcactatgaatgtgaaaactgtgccaaggttttcacggaccctagcaaccttcagcggcacattcgctctcagcatgtcggtgcccgggcccatgcatgcccggagtgtggcaaaacgtttgccacttcgtcgggcctcaaacaacacaagcacatccacagcagtgtgaagccctttatctgtgaggtctgccataaatcctatactcagttttcaaacctttgccgtcataagcgcatgcatgctgattgcagaacccaaatcaagtgcaaagactgtggacaaatgttcagcactacgtcttccttaaataaacacaggaggttttgtgagggcaagaaccattttgcggcaggtggattttttggccaaggcatttcacttcctggaaccccagctatggataaaacgtccatggttaatatgagtcatgccaacccgggccttgctgactattttggcgccaataggcatcctgctggtcttacctttccaacagctcctggattttcttttagcttccctggtctgtttccttccggcttgtaccacaggcctcctttgatacctgctagttctcctgttaaaggactatcaagtactgaacagacaaacaaaagtcaaagtcccctcatgacacatcctcagatactgccagctacacaggatattttgaaggcactatctaaacacccatctgtaggggacaataagccagtggagctccagcccgagaggtcctctgaagagaggccctttgagaaaatcagtgaccagtcagagagtagtgaccttgatgatgtcagtacaccaagtggcagtgacctggaaacaacctcgggctctgatctggaaagtgacattgaaagtgataaagagaaatttaaagaaaatggtaaaatgttcaaagacaaagtaagccctcttcagaatctggcttcaataaataataagaaagaatacagcaatcattccattttctcaccatctttagaggagcagactgcggtgtcaggagctgtgaatgattctataaaggctattgcttctattgctgaaaaatactttggttcaacaggactggtggggctgcaagacaaaaaagttggagctttaccttacccttccatgtttcccctcccattttttccagcattctctcaatcaatgtacccatttcctgatagagacttgagatcgttacctttgaaaatggaaccccaatcaccaggtgaagtaaagaaactgcagaagggcagctctgagtccccctttgatctcaccactaagcgaaaggatgagaagcccttgactccagtcccctccaagcctccagtgacacctgccacaagccaagaccagcccctggatctaagtatgggcagtaggagtagagccagtgggacaaagctgactgagcctcgaaaaaaccacgtgtttgggggaaaaaaaggaagcaacgtcgaatcaagacctgcttcagatggttccttgcagcatgcaagacccactcctttctttatggaccctatttacagagtagagaaaagaaaactaactgacccacttgaagctttaaaagagaaatacttgaggccttctccaggattcttgtttcacccacaaatgtcagctattgaaaacatggcagaaaagctagagagcttcagtgccctgaaacctgaggccagtgagctcttacagtcagtgccctctatgttcaacttcagggcgcctcccaatgccctgccagagaaccttctgcggaagggaaaggagcgctatacctgcagatactgtggcaagatttttccaaggtctgcaaacctaacacggcacttgagaacccacacaggagagcagccttacagatgcaaatactgtgacagatcatttagcatatcttctaacttgcaaaggcatgttcgcaacatccacaataaagagaagccatttaagtgtcacttatgtgataggtgttttggtcaacaaaccaatttagacagacacctaaagaaacatgagaatgggaacatgtccggtacagcaacatcgtcgcctcattctgaactggaaagtacaggtgcgattctggatgacaaagaagatgcttacttcacagaaattcgaaatttcattgggaacagcaaccatggcagccaatctcccaggaatgtggaggagagaatgaatggcagtcattttaaagatgaaaaggctttggtgaccagtcaaaattcagacttgctggatgatgaagaagttgaagatgaggtgttgttagatgaggaggatgaagacaatgatattactggaaaaacaggaaaggaaccagtgacaagtaatttacatgaaggaaaccctgaggatgactatgaagaaaccagtgccctggagatgagttgcaagacatccccagtgaggtataaagaggaagaatataaaagtggactttctgctctagatcatataaggcacttcacagatagcctcaaaatgaggaaaatggaagataatcaatattctgaagctgagctgtcttcttttagtacttcccatgtgccagaggaacttaagcagccgttacacagaaagtccaaatcgcaggcatatgctatgatgctgtcactgtctgacaaggagtccctccattctacatcccacagttcttccaacgtgtggcacagtatggccagggctgcggcggaatccagtgctatccagtccataagccacgtatgacgttatcaaggttgaccagagtgggaccaagtccaacagtagcatggctctttcatataggactatttacaagactgctgagcagaatgccttataaacctgcagggtcactcatctaaagtctagtgaccttaaactgaatgatttaaaaaagaaaagaaagaaaaaagaaactatttattctcgatattttgttttgcacagcaaaggcagctgctgacttctggaagatcaatcaatgcgacttaaagtgattcagtgaaaacaaaaaacttggtgggctgaaggcatcttccagtttaccccaccttagggtatgggtgggtgagaagggcagttgagatggcagcattgatatgaatgaacactccatagaaactgaattctcttttgtacaagatcacctgacatgattgggaacagttgcttttaattacagatttaatttttttcttcgttaaagttttatgtaatttaaccctttgaagacagaagtagttggatgaaatgcacagtcaattattatagaaactgataacagggagtacttgttcccccttttgccttcttaagtacattgtttaaaactagggaaaaagggtatgtgtatattgtaaactatggatgttaacactcaaagaggttaagtcagtgaagtaacctattcatcaccagtaccgctgtaccactaataaattgtttgccaaatccttgtaataacatcttaattttagacaatcatgtcactgtttttaatgtttatttttttgtgtgtgttgcgtgtatcatgtatttatttgttggcaaactattgtttgttgattaaaatagcactgttccagtcagccactactttatgacgtctgaggcacacccctttccgaatttcaaggaccaaggtgacccgacctgtgtatgagagtgccaaatggtgtttggcttttcttaacattcctttttgtttgtttgttttgttttccttcttaatgaactaaatacgaatagatgcaacttagtttttgtaatactgaaatcgattcaattgtataaacgattataatttctttcatggaagcatgattcttctgattaaaaactgtactccatattttatgctggttgtctgcaagcttgtgcgatgttatgttcatgttaatcctatttgtaaaatgaagtgttcccaaccttatgttaaaagagagaagtaaataacagactgtattcagttattttgccctttattgaggaaccagatttgttttctttttgtttgtaatctcattttgaaataatcagcaagttgaggtactttcttcaaatgctttgtacaatataaactgttatgcctttcagtgcattactatgggaggagcaactaaaaaataaagacttacaaaaaggagtattttt

SEQ ID NO: 22 - protein, 1042 aa, Homo sapiens
MKSEDYPHETMAPDIHEERQYRCEDCDQLFESKAELADHQKFPCSTPHSAFSMVEEDFQQKLESENDLQEIHTIQECKECDQVFPDLQSLEKHMLSHTEEREYKCDQCPKAFNWKSNLIRHQMSHDSGKHYECENCAKVFTDPSNLQRHIRSQHVGARAHACPECGKTFATSSGLKQHKHIHSSVKPFICEVCHKSYTQFSNLCRHKRMHADCRTQIKCKDCGQMFSTTSSLNKHRRFCEGKNHFAAGGFFGQGISLPGTPAMDKTSMVNMSHANPGLADYFGANRHPAGLTFPTAPGFSFSFPGLFPSGLYHRPPLIPASSPVKGLSSTEQTNKSQSPLMTHPQILPATQDILKALSKHPSVGDNKPVELQPERSSEERPFEKISDQSESSDLDDVSTPSGSDLETTSGSDLESDIESDKEKFKENGKMFKDKVSPLQNLASINNKKEYSNHSIFSPSLEEQTAVSGAVNDSIKAIASIAEKYFGSTGLVGLQDKKVGALPYPSMFPLPFFPAFSQSMYPFPDRDLRSLPLKMEPQSPGEVKKLQKGSSESPFDLTTKRKDEKPLTPVPSKPPVTPATSQDQPLDLSMGSRSRASGTKLTEPRKNHVFGGKKGSNVESRPASDGSLQHARPTPFFMDPIYRVEKRKLTDPLEALKEKYLRPSPGFLFHPQMSAIENMAEKLESFSALKPEASELLQSVPSMFNFRAPPNALPENLLRKGKERYTCRYCGKIFPRSANLTRHLRTHTGEQPYRCKYCDRSFSISSNLQRHVRNIHNKEKPFKCHLCDRCFGQQTNLDRHLKKHENGNMSGTATSSPHSELESTGAILDDKEDAYFTEIRNFIGNSNHGSQSPRNVEERMNGSHFKDEKALVTSQNSDLLDDEEVEDEVLLDEEDEDNDITGKTGKEPVTSNLHEGNPEDDYEETSALEMSCKTSPVRYKEEEYKSGLSALDHIRHFTDSLKMRKMEDNQYSEAELSSFSTSHVPEELKQPLHRKSKSQAYAMMLSLSDKESLHSTSHSSSNVWHSMARAAAESSAIQSISHV

SEQ ID NO: 23 - DNA, 1601 bp, Homo sapiens
  CDS (174)..(686)
agggtagagtgagaagcaccagcaggcagtaacagccaacccttagccattgctaagggcagagaactggtggagcctttctcttactcccaggacttcagcacctaagacagctccaaaacaaaccagaacagtcagctccgggggagcacgactgggcgagaggcacagaaatggacaccagaaataaggcccagctccttgtgctcctgactcttctcagtgtgctcttctcacagacttcggcatggcctctttacagggcaccttctgctctcaggttgggtgacagaataccctttgagggagcaaatgaacctgatcaagtttcattaaaagaagacattgacatgttgcaaaatgcattagctgaaaatgacacaccctattatgatgtatccagaaatgccaggcatgctgatggagttttcaccagtgacttcagtaaactcttgggtcaactttctgccaaaaagtaccttgagtctcttatgggaaaacgtgttagcagtaacatctcagaagaccctgtaccagtcaaacgtcactcagatgcagtcttcactgacaactatacccgccttagaaaacaaatggctgtaaagaaatatttgaactcaattctgaatggaaagaggagcagtgagggagaatctcccgactttccagaagagttagaaaaatgatgaaaaagacctttggagcaaagctgatgacaacttcccagtgaattcttgaaggaaaatgatacgcaacataattaaattttgagttctacataagtaattcaagaaaacaacttcaatatccaaaccaaataaaaatattgtgttgtgaatgttgtgatgtattctagctaatgtaataactgtgaagtttacattgtaaatagtatttgagagttctaaattttgtctttaactcataaaaagcctgcaatttcatatgctgtatatcctttctaacaaaaaaatatatttaatgataagtaaatgctaggttaattccaattatatgagacgtttttggaagagtagtaatagagcaaaattgatgtgtttatttatagagtgtacttaactattcaggagagtagaacagataatcagtgtgtctaaatttgaatgttaagcagatggaatgctgtgttaaataaacctcaaaatgtctaagatagtaacaatgaagataaaaagacattcttccaaaaagattttcagaaaatattatgtgtttccatattttataggcaacctttatttttaatggtgttttaaaaaatctcaaatttggattgctaatcaccaaaggctctctcctgatagtctttcagttaaggagaacgacccctgcttctgacactgaaacttccctttctgcttgtgttaagtatgtgtaaaatgtgaagtgaatgaaacactcagttgttcaataataaatatttttgccataatgactcagaatattgctttggtcatatgagcttccttctgtgaaagtacatttggagacacaactatttttccaaaataattttaagaaatcaaagagagaaaataaagaccttgcttatgattgcagataaaaaaaaaaaaaaaaaa

SEQ ID NO: 24 - protein, 170 aa, Homo sapiens
MDTRNKAQLLVLLTLLSVLFSQTSAWPLYRAPSALRLGDRIPFEGANEPDQVSLKEDIDMLQNALAENDTPYYDVSRNARHADGVFTSDFSKLLGQLSAKKYLESLMGKRVSSNISEDPVPVKRHSDAVFTDNYTRLRKQMAVKKYLNSILNGKRSSEGESPDFPEELEK

SEQ ID NO: 25 - DNA, 5132 bp, Homo sapiens
  CDS (28)..(4848)
gcgccgcgctgcgcaccgccgccgaccatgagcgaggacagccgcggagacagccgcgccgagagcgccaaggacctggagaagcagcttcgcctgcgcgtgtgcgtgctcagcgagctccagaagaccgagcgggactatgtgggcacgctggagttcctggtgtcggcattcttacacagaatgaaccagtgtgcagcatcaaaagttgacaaaaatgtgacagaagaaacagtgaagatgttgttctcaaacattgaagacatccttgcagtacataaagaattcttaaaagtcgtggaagaatgcttacaccccgaacctaatgctcaacaagaagtgggaacctgctttcttcactttaaagacaagtttcgtatctatgatgaatattgtagtaaccatgagaaggcacaaaaattacttcttgaactcaacaaaataagaacaatccggacatttcttttgaactgcatgctgcttggaggacggaagaacacagatgttcccttggaaggatatttagtaacaccaatacaaagaatatgcaagtaccctcttattttgaaggagttgctgaagcggactccacggaaacacagtgactatgcagcagtgatggaagccctccaagccatgaaagctgtctgttccaacataaacgaggccaagagacagatggagaagttagaagttttagaggaatggcagtctcacattgaaggctgggaggggtccaacatcactgacacctgcactgaaatgctaatgtgtggagtcttactgaaaatttcttctggaaatattcaagaacgggtgttttttcttttcgataatcttttggtgtactgcaaaagaaaacacagacggttgaagaacagcaaggcatctacagatggacatcggtacctttttcgtggccggatcaacacggaggtgatggaagtggagaatgtggatgatggcaccgctgatttccatagcagtggacacattgttgttaatggatggaagatacataacacagcaaaaaataaatggtttgtttgtatggcaaaaacacctgaagagaagcatgaatggtttgaagctattttgaaagaaagagaacggcggaaaggtttaaaattaggaatggagcaagatacctgggtcatgatctctgaacagggtgagaaactttataaaatgatgtgcagacaaggaaatctgatcaaagaccgaaagagaaaactgactacgttccctaaatgctttcttggaagcgaatttgtgtcatggctgttggaaattggagagattcacaggcctgaggaaggcgtgcacttgggacaagcattattagaaaatggaatcattcaccatgttactgataaacatcaattcaaaccagaacagatgttatatagatttcgctatgatgatggaacattttatccaagaaatgagatgcaggacgtgatttcaaagggtgtaagattatattgtcgtcttcatagcctttttactccagtgataagagataaagattaccatttaaggacctacaaatctgtggtcatggccaacaaactgatagactggttaattgcacagggagattgccgcaccagagaagaggcaatgatatttggcgttggactctgtgacaatggatttatgcaccatgtccttgaaaaaagcgaattcaaagatgaacccctacttttccgttttttttcggatgaggaaatggagggatcaaatatgaaacatcgacttatgaaacatgacttaaaagttgtggaaaatgttatagctaagtcattattgattaaatccaatgaaggcagctatggctttggattagaagacaaaaataaagttccaataataaagttggtagaaaagggatctaatgctgagatggctggcatggaagtcgggaaaaagatttttgctattaatggtgacctagtttttatgagacctttcaatgaagtggattgcttcctgaaatcgtgtttaaacagcagaaaacctctaagagttcttgtgagcacaaagccaagagagacagtgaaaattccagattcagctgatggacttggcttccagatccggggatttggcccttctgttgtgcatgctgtaggaagaggaactgtggctgcagcagctggtcttcaccctggacagtgcattatcaaggtgaatggaatcaatgtcagcaaagagacacatgccagtgtcattgcacacgttacagcctgcaggaagtacaggcggccaacgaagcaagattccatacaatgggtttataatagcattgagagtgctcaagaagaccttcaaaaatctcactccaagccccctggagatgaagcaggggatgcttttgactgtaaagtagaagaggttattgacaaattcaacactatggccatcattgatgggaagaaggagcatgtgagtctgacagtggacaatgtccacctggaatatggtgtcgtgtatgagtacgacagcacagctggcatcaagtgcaatgtggtggaaaagatgattgagcccaaaggtttcttcagcttaactgccaagattcttgaagccctggctaaaagtgatgagcattttgtacaaaactgtaccagcctaaattctctaaatgaagtgattcctactgaccttcagagtaaattcagtgccctttgcagtgaaagaattgaacacctatgtcagagaatatccagttataaaaagttttctcgtgtactgaagaatagggcctggcctacttttaaacaggccaaatctaaaatctccccactgcacagcagtgatttctgccctaccaactgccatgtcaatgtgatggaagtttcttatcccaaaacatcaacctctttgggaagtgcatttggtgttcagttggatagcaggaagcataattctcatgataaagaaaacaaatcttcggagcaagggaaactgagccctatggtgtacattcagcacaccatcacaaccatggcggccccttcaggtctgtctctgggacagcaggatggccatggtctcaggtatctgctaaaagaagaagacttagaaacccaagacatctatcagaaactgctgggcaaacttcagactgcactgaaagaggtggagatgtgtgtttgtcaaatagatgaccttctgtcttcaataacatattctcctaaattagaacgtaagacatcagagggcataataccaacagacagtgacaatgagaagggagaaagaaacagcaaacgggtatgttttaatgtagcaggagatgaacaggaagattctggtcatgacaccatcagcaacagagactcttacagtgactgcaacagcaataggaattccatcgcctccttcaccagcatctgcagcagccagtgcagctcgtatttccacagtgatgaaatggactcaggtgatgaacttcccttaagtgttcgcatatctcatgataaacaggacaagatacatagttgccttgagcatcttttcagccaggtggattcaattaccaatctcctaaaagggcaggctgttgtgagggcctttgaccaaaccaagtatctcactccaggccgaggattgcaagaatttcaacaggaaatggaaccaaagctgagttgtccaaaaaggctacggcttcatatcaagcaagatccttggaatcttcccagcagcgtccggactcttgctcagaacatcaggaaatttgttgaagaggtaaagtgtaggctactcctggctcttcttgaatattcagatagtgagacacagctccgtagagacatggttttctgccagactcttgtggccactgtctgtgccttctctgagcagctcatggcggccttgaaccagatgtttgacaacagcaaggaaaatgagatggaaacttgggaagccagcaggaggtggctggaccagatagcgaatgcaggtgttctttttcactttcagtcacttctgtcaccaaacttgacagatgaacaagccatgttagaagatacactggttgcactatttgatttggaaaaggtttccttttactttaaaccatcagaagaggaacctctggttgcaaatgttcctcttacatatcaagcagaaggaagtcggcaagctctgaaagtttacttctacattgatagttatcattttgaacaacttcctcaacggctgaaaaatggaggagggtttaaaattcatcctgttctttttgcacaagcactagagagcatggaaggatattattacagagacaatgtttctgtggaagaatttcaagctcagataaatgcagcctcactggaaaaggtcaaacagtacaaccagaagctcagggcattctacttggacaagtcaaattcaccaccaaactccacatccaaagctgcctatgtagataagctaatgaggcctctcaacgctttggatgaactttaccgactggtagcctcgtttatcagatccaagcgcacagctgcctgtgcaaacacagcttgcagtgcttctggggttggactgctgtcagtttcctcggagctgtgcaacaggctgggcgcctgccacatcatcatgtgcagcagcggtgtgcatcggtgcaccctgagcgtgacgctggagcaagccatcattctggccagaagccacggactgccacctcgttacatcatgcaggctacagatgtgatgcggaagcagggagcaagagttcagaacacagcgaagaatttgggagtcagagaccggactccacagtctgcaccaaggctgtacaagctgtgcgagccacctcccccagctggagaagaatgaaaagaactcccaagaaaccaggcaggcagaagctcctgaatgctggactagacaaactacatgctggctaaacattctccactgaagatacatcaatgcttttttttttttttttttctgtaaatctttcttcccattgcaaacaagtagtgatcagtttatactttgatatttatgctggaaatggatagaagttcaatcagacagttcagcttcacgaactgatgtctctctgtattgacattaagtattcacttttagagtaaaatccatccctgggacat

SEQ ID NO: 26 - protein, 1606 aa, Homo sapiens
MSEDSRGDSRAESAKDLEKQLRLRVCVLSELQKTERDYVGTLEFLVSAFLHRMNQCAASKVDKNVTEETVKMLFSNIEDILAVHKEFLKVVEECLHPEPNAQQEVGTCFLHFKDKFRIYDEYCSNHEKAQKLLLELNKIRTIRTFLLNCMLLGGRKNTDVPLEGYLVTPIQRICKYPLILKELLKRTPRKHSDYAAVMEALQAMKAVCSNINEAKRQMEKLEVLEEWQSHIEGWEGSNITDTCTEMLMCGVLLKISSGNIQERVFFLFDNLLVYCKRKHRRLKNSKASTDGHRYLFRGRINTEVMEVENVDDGTADFHSSGHIVVNGWKIHNTAKNKWFVCMAKTPEEKHEWFEAILKERERRKGLKLGMEQDTWVMISEQGEKLYKMMCRQGNLIKDRKRKLTTFPKCFLGSEFVSWLLEIGEIHRPEEGVHLGQALLENGIIHHVTDKHQFKPEQMLYRFRYDDGTFYPRNEMQDVISKGVRLYCRLHSLFTPVIRDKDYHLRTYKSVVMANKLIDWLIAQGDCRTREEAMIFGVGLCDNGFMHHVLEKSEFKDEPLLFRFFSDEEMEGSNMKHRLMKHDLKVVENVIAKSLLIKSNEGSYGFGLEDKNKVPIIKLVEKGSNAEMAGMEVGKKIFAINGDLVFMRPFNEVDCFLKSCLNSRKPLRVLVSTKPRETVKIPDSADGLGFQIRGFGPSVVHAVGRGTVAAAAGLHPGQCIIKVNGINVSKETHASVIAHVTACRKYRRPTKQDSIQWVYNSIESAQEDLQKSHSKPPGDEAGDAFDCKVEEVIDKFNTMAIIDGKKEHVSLTVDNVHLEYGVVYEYDSTAGIKCNVVEKMIEPKGFFSLTAKILEALAKSDEHFVQNCTSLNSLNEVIPTDLQSKFSALCSERIEHLCQRISSYKKFSRVLKNRAWPTFKQAKSKISPLHSSDFCPTNCHVNVMEVSYPKTSTSLGSAFGVQLDSRKHNSHDKENKSSEQGKLSPMVYIQHTITTMAAPSGLSLGQQDGHGLRYLLKEEDLETQDIYQKLLGKLQTALKEVEMCVCQIDDLLSSITYSPKLERKTSEGIIPTDSDNEKGERNSKRVCFNVAGDEQEDSGHDTISNRDSYSDCNSNRNSIASFTSICSSQCSSYFHSDEMDSGDELPLSVRISHDKQDKIHSCLEHLFSQVDSITNLLKGQAVVRAFDQTKYLTPGRGLQEFQQEMEPKLSCPKRLRLHIKQDPWNLPSSVRTLAQNIRKFVEEVKCRLLLALLEYSDSETQLRRDMVFCQTLVATVCAFSEQLMAALNQMFDNSKENEMETWEASRRWLDQIANAGVLFHFQSLLSPNLTDEQAMLEDTLVALFDLEKVSFYFKPSEEEPLVANVPLTYQAEGSRQALKVYFYIDSYHFEQLPQRLKNGGGFKIHPVLFAQALESMEGYYYRDNVSVEEFQAQINAASLEKVKQYNQKLRAFYLDKSNSPPNSTSKAAYVDKLMRPLNALDELYRLVASFIRSKRTAACANTACSASGVGLLSVSSELCNRLGACHIIMCSSGVHRCTLSVTLEQAIILARSHGLPPRYIMQATDVMRKQGARVQNTAKNLGVRDRTPQSAPRLYKLCEPPPPAGEE

SEQ ID NO: 27 - DNA, 3066 bp, Homo sapiens
  CDS (9)..(716)
ttccttttatgcgaacacaagtatatgaggggttgtgtaaaaattatttttctcttgctgtactacaaagagatagaatcaaactgctttttttcgacatactggtttttctttctgtttttcttctctttcttctatttcttgtggatattatggctaataacacaacaagtttagggagtccatggccagaaaacttttgggaggaccttatcatgtccttcactgtatccatggcaatcgggctggtacttggaggatttatttgggctgtgttcatttgtctgtctcgaagaagaagagccagtgctcccatctcacagtggagttcaagcaggagatctaggtcttcttacacccacggcctcaacagaactggattttaccgccacagtggctgtgaacgtcgaagcaacctcagcctggccagtctcaccttccagcgacaagcttccctggaacaagcaaattcctttccaagaaaatcaagtttcagagcttctactttccatccctttctgcaatgtccaccacttcctgtggaaactgagagtcagctggtgactctcccttcttccaatatctctcccaccatcagcacttcccacagtctgagccgtcctgactactggtccagtaacagtcttcgagtgggcctttcaacaccgcccccacctgcctatgagtccatcatcaaggcattcccagattcctgagtagggtggcttttggtttttgtttctttcttgtcttgtcttttattgaaaggaaatcaaaaataggctaaacagaattttgagggcatggcccaaataactcatgagttccaagttgaaacatggttgtgcaagttggacattacaatgtaaaacacattttcttcaaacacgttttcccttttgtttcaaaaaatgtaatattttcccccaagcgttttatatttatgtattttgtattcaatgtgaggcttattaaaaatagtgattctaatgtaagaatcagctaagatgcattatatatattttaattaaaattaaaacttcagatatttgtggattacaatcctcatttacttccaatgtgactaaaaagagaaaaaaaatcactgtgtcactttaaagaaaaatcttctaagggatttggattttactttctttagaatgacaagtgaatcatattgacattttacaatcttagatttttctttttttttcttttgagacagggtcttgatccgtcgcccaggcgggagttgcagtagcatgatcaggactcactgcagcctctatctcccaggctcaagtaatcctcccatcttagtgccccaagtagctgggactacaggggtgcactaccacaccgggttgaatttttttttaattttagtagagatgaagtgtcactatgttaccaaggctggtctcaaactcctaaactcagatgatcctcctgcctcggcctcccaaagtgctggaattagcctggccaatcttggatttttaatggaatatgtgggcacaaaatgacagaacataggacattctaaagttccttgatttgatcattataagaagtgtgggactcaagcacaggaaactgaactcttttggtgtcattggatgtttcatttttgacactaattttttctggacaaactctttatgtgtttttcccaagaatagttatctacttcctggaggcaaaatccttggatttactaacatgatgatttaccttttcttcaccgttgtcgttacattgttagaaaagcaacaggaaaaaatccaattcatttgacctaaaaacaagcctcaagtttaaaaccaagctcacgtttttcttaagggaaaaattttctttcttaaacttacatctagcaacttggaaagcactttctctggggatcttcttttgtaactttgcagacaaataagtatgagtcactggggagagagtttgttattgaaatagatgttgcccatgaagaattctccttcctggattgactcttaatcatcaggcatcattcctggtttgcttctctacgaatctcaattccaacttctctgcagagtctgtacagtgattaagccatgccagatggtctttggtgcacacagttatttaagaatccacttccacaggtggctgcccttgtaaggaagaatgcatccctaaatgtggccaccagagagttccagtgggcagatgtctgtggctgcccttctcatttaaggacatgagttcactggagtattactcaaaaagtctgtggttcatttccagtattgtgaatatttagtttatgtggccgtttctttgtttctttgaacagtgggattttcagtgaaaaagtaccctctttttcatttcctattgcagtggtcacagctaatagtgtctgaacatggttcaagaataagagattccatgtagcattttctttattattttcatttcccttatattatccatcattccttaaggacaattattcttaataatgcttatagaaaatgttctctaattaaacatgccaaaaggaaaaagtaagagaaagagggagcaagaagaaaatggaagaaaaagggaaaaaagctaaccggataaccaatttgttataagttggttttcaacaaagaaatttagcagccaagtaaggtttcaagggaatattaacttggtatcagggctacttttttttttttttttttacttgcatgtcatccttaatgtctaacatgaaaaatcagcaaagagtatggtttttatcaagaatttgtgttgggagtaaaaactgctttatagctcccaaattaggaagagaagagcagaaatcctctggggcatttaaccatctggcagaattgttgctgcacccttatcccagttataagacagtcaaaatgactatttcctaaatattgtgagtgtatgaaatgtgaaattaaagcaaaaactggagacttttaatgtatttctttaatttgaaatgttttgtggattgtgaaataaaaataaatttatgtcaagttttattcaaaaaaaaaaaaaaaaaaaaaaaaa

SEQ ID NO: 28 - protein, 235 aa, Homo sapiens
MRTQVYEGLCKNYFSLAVLQRDRIKLLFFDILVFLSVFLLFLLFLVDIMANNTTSLGSPWPENFWEDLIMSFTVSMAIGLVLGGFIWAVFICLSRRRRASAPISQWSSSRRSRSSYTHGLNRTGFYRHSGCERRSNLSLASLTFQRQASLEQANSFPRKSSFRASTFHPFLQCPPLPVETESQLVTLPSSNISPTISTSHSLSRPDYWSSNSLRVGLSTPPPPAYESIIKAFPDS

SEQ ID NO: 29 - DNA, 831 bp, Homo sapiens
  CDS (18)..(761)
aggcacactctaccaccatgaatccactcctgatccttacctttgtggcagctgctcttgctgccccctttgatgatgatgacaagatcgttgggggctacaactgtgaggagaattctgtcccctaccaggtgtccctgaattctggctaccacttctgtggtggctccctcatcaacgaacagtgggtggtatcagcaggccactgctacaagtcccgcatccaggtgagactgggagagcacaacatcgaagtcctggaggggaatgagcagttcatcaatgcagccaagatcatccgccacccccaatacgacaggaagactctgaacaatgacatcatgttaatcaagctctcctcacgtgcagtaatcaacgcccgcgtgtccaccatctctctgcccaccgcccctccagccactggcacgaagtgcctcatctctggctggggcaacactgcgagctctggcgccgactacccagacgagctgcagtgcctggatgctcctgtgctgagccaggctaagtgtgaagcctcctaccctggaaagattaccagcaacatgttctgtgtgggcttccttgagggaggcaaggattcatgtcagggtgattctggtggccctgtggtctgcaatggacagctccaaggagttgtctcctggggtgatggctgtgcccagaagaacaagcctggagtctacaccaaggtctacaactatgtgaaatggattaagaacaccatagctgccaatagctaaagcccccagtatctcttcagtctctataccaataaagtgaccctgttctcactgtcaaaaaaaaaaaaaa

SEQ ID NO: 30 - protein, 247 aa, Homo sapiens
MNPLLILTFVAAALAAPFDDDDKIVGGYNCEENSVPYQVSLNSGYHFCGGSLINEQWVVSAGHCYKSRIQVRLGEHNIEVLEGNEQFINAAKIIRHPQYDRKTLNNDIMLIKLSSRAVINARVSTISLPTAPPATGTKCLISGWGNTASSGADYPDELQCLDAPVLSQAKCEASYPGKITSNMFCVGFLEGGKDSCQGDSGGPVVCNGQLQGVVSWGDGCAQKNKPGVYTKVYNYVKWIKNTIAANS

SEQ ID NO: 31 - DNA, 802 bp, Homo sapiens
  CDS (7)..(750)
accaccatgaatctacttctgatccttacctttgttgcagctgctgttgctgccccctttgatgatgatgacaagatcgttgggggctacatctgtgaggagaattctgtcccctaccaggtgtccttgaattctggctaccacttctgcggtggctccctcatcagcgaacagtgggtggtgtcagcaggtcactgctacaagtcccgcatccaggtgagactgggagagcacaacatcgaagtcctggaggggaatgaacagttcatcaatgcagccaagatcatccgccaccccaaatacaacagccggactctggacaatgacatcctgctgatcaagctctcctcacctgccgtcatcaattcccgcgtgtccgccatctctctgcccactgcccctccagctgctggcaccgagtccctcatctccggctggggcaacactctgagttctggtgccgactacccagacgagctgcagtgcctggatgctcctgtgctgagccaggctgagtgtgaagcctcctaccctggaaagattaccaacaacatgttctgtgtgggcttcctcgagggaggcaaggattcctgccagggtgattctggtggccctgtggtctccaatggagagctccaaggaattgtctcctggggctatggctgtgcccagaagaacaggcctggagtctacaccaaggtctacaactatgtggactggattaaggacaccatagctgccaacagctaaagcccctggtccctctgcagtctctataccaataaagtgaccctgctctcac

SEQ ID NO: 32 - protein, 247 aa, Homo sapiens
MNLLLILTFVAAAVAAPFDDDDKIVGGYICEENSVPYQVSLNSGYHFCGGSLISEQWVVSAGHCYKSRIQVRLGEHNIEVLEGNEQFINAAKIIRHPKYNSRTLDNDILLIKLSSPAVINSRVSAISLPTAPPAAGTESLISGWGNTLSSGADYPDELQCLDAPVLSQAECEASYPGKITNNMFCVGFLEGGKDSCQGDSGGPVVSNGELQGIVSWGYGCAQKNRPGVYTKVYNYVDWIKDTIAANS

SEQ ID NO: 33 - DNA, 908 bp, Homo sapiens
  CDS (45)..(908)
agctggaggccaggggagaaactccagaaggagagacatttgccatggctgagcacttcaaacagatcattagatgtcctgtctgtctaaaagatcttgaagaagccgtgcaactgaaatgtggatatgcctgctgcctccagtgcctcaattcactccagaaggagcccgatggggaaggtttactgtgccgtttctgctctgtggtctctcagaaggatgacatcaagcccaagtacaagctgagggcgctggtttccatcatcaaggaactagagcccaagctgaaatctgttctaacaatgaacccaaggatgaggaagtttcaagtggatatgacgttcgatgtggacacagccaacaactatctcatcatttctgaagacctgaggagtttccgaagtggggatttgagccagaataggaaggagcaagctgagaggttcgacactgccctgtgcgtcctgggcacccctcgcttcacttccggccgccattactgggaggtggacgtgggcaccagccaagtgtgggatgtgggcgtgtgcaaggaatctgtgaaccgacaggggaagattgtgctttcttcagaacacggcttcttgactgtgggttgcagagaaggaaaggtctttgctgccagcactgtgcctatgactcctctctgggtgagtccccagttgcacagagtggggattttcctggatgtaggtatgaggtccattgccttttacaatgttagtgatgggtgccatatctacacattcatcgagattcctgtttgcgagccctggcgtccattttttgctcataaacgtggaagtcaagatgatcagagcatcctgagtatctgttctgtgatcaatccatccgctgccagtgccccagtttcttctgagggaaagtaa

SEQ ID NO: 34 - protein, 287 aa, Homo sapiens
MAEHFKQIIRCPVCLKDLEEAVQLKCGYACCLQCLNSLQKEPDGEGLLCRFCSVVSQKDDIKPKYKLRALVSIIKELEPKLKSVLTMNPRMRKFQVDMTFDVDTANNYLIISEDLRSFRSGDLSQNRKEQAERFDTALCVLGTPRFTSGRHYWEVDVGTSQVWDVGVCKESVNRQGKIVLSSEHGFLTVGCREGKVFAASTVPMTPLWVSPQLHRVGIFLDVGMRSIAFYNVSDGCHIYTFIEIPVCEPWRPFFAHKRGSQDDQSILSICSVINPSAASAPVSSEGK

SEQ ID NO: 35 - DNA, 2720 bp, Homo sapiens
  CDS (148)..(1266)
agaggggcgggagctctggctcaggtaaaaactctttcttcggctcgcgagctgagaggagcaggtagaggggcagaggcgggactgtcgtctgggggagccgcccaggaggctcctcaggccgaccccagaccctggctggccaggatgaagtatctccggcaccggcggcccaatgccaccctcattctggccatcggcgctttcaccctcctcctcttcagtctgctagtgtcaccacccacctgcaaggtccaggagcagccaccggcgatccccgaggccctggcctggcccactccacccacccgcccagccccggccccgtgccatgccaacacctctatggtcacccacccggacttcgccacgcagccgcagcacgttcagaacttcctcctgtacagacactgccgccactttcccctgctgcaggacgtgcccccctctaagtgcgcgcagccggtcttcctgctgctggtgatcaagtcctcccctagcaactatgtgcgccgcgagctgctgcggcgcacgtggggccgcgagcgcaaggtacggggtttgcagctgcgcctcctcttcctggtgggcacagcctccaacccgcacgaggcccgcaaggtcaaccggctgctggagctggaggcacagactcacggagacatcctgcagtgggacttccacgactccttcttcaacctcacgctcaagcaggtcctgttcttacagtggcaggagacaaggtgcgccaacgccagcttcgtgctcaacggggatgatgacgtctttgcacacacagacaacatggtcttctacctgcaggaccatgaccctggccgccacctcttcgtggggcaactgatccaaaacgtgggccccatccgggctttttggagcaagtactatgtgccagaggtggtgactcagaatgagcggtacccaccctattgtgggggtggtggcttcttgctgtcccgcttcacggccgctgccctgcgccgtgctgcccatgtcttggacatcttccccattgatgatgtcttcctgggtatgtgtctggagcttgagggactgaagcctgcctcccacagcggcatccgcacgtctggcgtgcgggctccatcgcaacgcctgtcctcctttgacccctgcttctaccgagacctgctgctggtgcaccgcttcctaccttatgagatgctgctcatgtgggatgcgctgaaccagcccaacctcacctgcggcaatcagacacagatctactgagtcagcatcagggtccccagcctctgggctcctgtttccataggaaggggcgacaccttcctcccaggaagctgagacctttgtggtctgagcataagggagtgccagggaaggtttgaggtttgatgagtgaatattctggctggcgaactcctacacatccttcaaaacccacctggtactgttccagcatcttccctggatggctggaggaactccagaaaatatccatcttctttttgtggctgctaatggcagaagtgcctgtgctagagttccaactgtggatgcatccgtcccgtttgagtcaaagtcttacttccctgctctcacctactcacagacgggatgctaagcagtgcacctgcagtggtttaatggcagataagctccgtctgcagttccaggccagccagaaactcctgtgtccacatagagctgacgtgagaaatatctttcagcccaggagagaggggtcctgatcttaaccctttcctgggtctcagacaactcagaaggttggggggataccagagaggtggtggaataggaccgccccctccttacttgtgggatcaaatgctgtaatggtggaggtgtgggcagaggagggaggcaagtgtcctttgaaagttgtgagagctcagagtttctggggtcctcattaggagcccccatccctgtgttccccaagaattcagagaacagcactggggctggaatgatctttaatgggcccaaggccaacaggcatatgcctcactactgcctggagaagggagagattcaggtcctccagcagcctccctcacccagtatgttttacagattacggggggaccgggtgagccagtgaccccctgtagcccccagcttcaggcctcagtgtctgccagtcaagcttcacaggcattgtgatggggcagccttggggaatataaaattttgtgaagacttggagatctttttttttttttaagcaaatttacaagtttcaacagacaagtccacattcatccctaaaagtctcattttccagtagaaaatatacactggtaaaaacggggcatggggccgtggctcagggctgtaattctagcacattgggagaccaaagtgggaggatcacttgagcccaggagttctggatcctgtctctgcacaaaataaaaaattactcaggcgtggtggtgctcacatgcctgtagtcccagctatacttgggaggctgaggcgagaggatcgcttgagcccaggagttggaggctgcagtgaaccatgattgcgccactgtactccactgggcggcaataagaagacaaaaacataaaacaggacatgtgtgaggcaaaagctgcaggaatttctatcaggcagatctgacctcatcccacccaccccctgctcagatacccttcatagctccttattgctttcagctcataaccccacat

SEQ ID NO: 36 - protein, 372 aa, Homo sapiens
MKYLRHRRPNATLILAIGAFTLLLFSLLVSPPTCKVQEQPPAIPEALAWPTPPTRPAPAPCHANTSMVTHPDFATQPQHVQNFLLYRHCRHFPLLQDVPPSKCAQPVFLLLVIKSSPSNYVRRELLRRTWGRERKVRGLQLRLLFLVGTASNPHEARKVNRLLELEAQTHGDILQWDFHDSFFNLTLKQVLFLQWQETRCANASFVLNGDDDVFAHTDNMVFYLQDHDPGRHLFVGQLIQNVGPIRAFWSKYYVPEVVTQNERYPPYCGGGGFLLSRFTAAALRRAAHVLDIFPIDDVFLGMCLELEGLKPASHSGIRTSGVRAPSQRLSSFDPCFYRDLLLVHRFLPYEMLLMWDALNQPNLTCGNQTQIY

SEQ ID NO: 37 - DNA, 2161 bp, Homo sapiens
  CDS (168)..(917)
tttttaacttgtttaactaagggtggctcctgttgagaggtgacagcgtgttggcagccctcgcagccctcgcttgctcttggtgcctcctcagcctcggcgcccactctggccgcacttgaggagcccttcagcccgctgctgcactgtgggagtcccttcctgggatggccgaggccggagccggctccctcagccttccgggaggaaaagccatggagacttcagcatcctcctcccagcctcaggacaacagtcaagtccacagagaaacagaagatgtagactatggagagacagatttccacaagcaagatgggaaggctggactcttttcccaagaacaatatgagagaaacaagtcttcttcctcctcctcctcttcctcctcatcctcctcatcttcttcatcctcctcctcctcagagagcaatgatgaggaccagcaacccagagcaaccggaaaacatcgacggagcctgggggctggatacccccacgggaacggctcacccggtcctgggcatggggagcctgacgttttgaaggatgagcttcaactctatggagatgctcctggagaggtggtaccctctggggaatcaggactccgaaggagaggctctgacccagcaagtggagaagtggaggcctctcagttaagaagactgaatataaagaaagatgatgagtttttccatttcgtcctcctgtgctttgccatcggggccttgctggtgtgttatcactattacgcagactggttcatgtctcttggggtcggcctgctcaccttcgcctccctggaaaccgttggcatctacttcggactagtgtaccgtatccacagcgtcctccaaggcttcatccccctcttccagaagtttaggctgacagggttcaggaagactgactgaggccacttccaggtgggcagcagaggcaggccccagtgtgaccaccactgcgacccctgagcccacaagggcagagcagcattctgagagacgcacaggagaccaagccagaccaataaacagaacacttttccttccatgtggtctgaatgttggcaccagcccgggcaggggcatctcatttgggcagtactgctgtgcaacccagctgcaaggatggaaggcagagggtgggtgtggggcctgaggcttcacagtacctggaccagcaggaagattctgggaggtcactgctctcagaggacagcaagggaccctgagctctgcaagctgtgatctgtctgggttcatggtttttctcaaatcccaggctatctgcatgcgctctcaggtgctaccgagccatcctgggagagatggatggtccactgctttgaggcagggagccatcgggctggggccccttggtgaacctgatgcaggtaagatgctgaggactaaaaccattttttttgcacccaaaaaaaaaggcaggaaaatgatcatcagaaactaaatggcagccaggcatgggggctcacaactgtaatcctcgcactttgggaggctcaggctaagggtcgcttgaagctgagagttcaagaccaacctgggcaacatagtgagacccccatctctacaatttttttttaatgaccaaatgtggcggtacatacctgtacatacctgcggttccagctactcaagaggctgaggcaggaggactgcttgagcccaggagttcagggctgcagtgaggtacgatcaagccactgcactccagcctgggcgacagagcaagatcgtttctctaaaattaaaaaaaaaaaaaaaagacaaataaaaattgcatgtatttgtagtgtacaacatgttttgaaatatgtggaatggctaaatcaagctaattaacatatgtatccctcacataccctttttttatgatgagaacagtaaaaacctactgtcagcaatttgcaaagtatacaatacattgttattaactatggtcaccatgctatgtgattgatatcctgaacttgaactgagttcttgatggtcacagacatgctgttctcaaaactcagaagggtaacttctgaggttttgatttcaagtacttgtatgtaacctaatttaacgggagcccagagtgcacagatcagatatttattaaacatataaataaaacacagttttcacga

SEQ ID NO: 38 - protein, 249 aa, Homo sapiens
MAEAGAGSLSLPGGKAMETSASSSQPQDNSQVHRETEDVDYGETDFHKQDGKAGLFSQEQYERNKSSSSSSSSSSSSSSSSSSSSSESNDEDQQPRATGKHRRSLGAGYPHGNGSPGPGHGEPDVLKDELQLYGDAPGEVVPSGESGLRRRGSDPASGEVEASQLRRLNIKKDDEFFHFVLLCFAIGALLVCYHYYADWFMSLGVGLLTFASLETVGIYFGLVYRIHSVLQGFIPLFQKFRLTGFRKTD

SEQ ID NO: 39 - DNA, 1812 bp, Homo sapiens
  CDS (409)..(1596)
aaagcttcccgagagccatggttggtccaggcaggcagtcggagcacctcgtggagcggctgctaacccagggtggggtggggtggggtggaggggtctgcttgtagtactctcagtcctgatgtcacaggcgcggcaaggacatcgcaaggaggagtcggattacagtaaggatgggcagtgcagcaggcagcccgagcgcactctccagccgccggggatcttagccctggaaaggcagcctgcaaggcgctcatcccgcggaggaggcacgctctcggcgctcactgctctccacgccggggacatttccaccgaatgtagcatgaaagggctctgctctacgtgccagccttgggtgagagctgatgctgaagacaccgcggtgggattccagctgtctgattaatgagaaacataatgtattttggtggtacatgccagagtcctgctctcccggcccttgtccgtccaccagcccctcctttgcaaccatcgctggatattaaaccatttcttccctttcctcttgacactgcagctgcagtcaacctcttccccaatttcaatgcgatggacccgattcagaaagctgtaataaaccatacattcggggttcctcttccccaccgaagaaagcaaatcatatcatgcaacatttgccagttgagatttaattctgatagccaggctgcggcccactacaaaggcacgaaacatgccaagaagctcaaagcactggaagccatgaaaaataagcagaaatctgtaactgccaaggacagcgcaaagactaccttcacctccatcactaccaataccatcaataccagctctgacaaaacagacggtactgcagggacaccagcaatatcaacgacgacaactgtggaaatccgcaaaagcagtgttatgacaactgagatcacctctaaagtggaaaaaagcccaacgacagccactggcaatagctcatgtccttctactgagaccgaggaagaaaaggcaaaacggcttctttactgttcgctatgcaaggttgctgtcaactctgcctcgcagctggaggcgcacaacagtggtactaagcacaaaaccatgttagaagcccggaatggaagtggcactatcaaagcctttcctagggcaggagtgaaaggcaaaggacctgttaataaaggaaacacaggcctccaaaataaaacatttcactgtgaaatctgtgatgtgcacgtcaactcggaaacgcaacttaaacagcacattagcagtagaaggcacaaagacagagctgctgggaagcccccgaaacctaaatacagtccttacaacaaactacagaagacagcacatccactgggggtaaaattagtattttcaaaagaaccttcaaagccattggctccacgaattctaccaaatcctctagcagctgcagcagccgcagcagcagtggcagtgagttcccccttcagtcttcgaactgctccagcagcaacactgttccagacttccgcgcttcctccggcactcctgcggccagctcctggacccattcggaccgcccacactcctgtgctgtttgctccttactaaattccaaataggagtaattgtactgcaataatttttcaaaaaacaaaaaacaaaacaaacaaaagagaactatgcagtgtttatttatagtttaaagtatatctgaagagccaggacttttgatagtgaattgaaaaggttataaaaaagtgggggaggtttgggggtgggagggagggagtggtattttctccaaattaaagcattcctcttgaa

SEQ ID NO: 40 - protein, 395 aa, Homo sapiens
MRNIMYFGGTCQSPALPALVRPPAPPLQPSLDIKPFLPFPLDTAAAVNLFPNFNAMDPIQKAVINHTFGVPLPHRRKQIISCNICQLRFNSDSQAAAHYKGTKHAKKLKALEAMKNKQKSVTAKDSAKTTFTSITTNTINTSSDKTDGTAGTPAISTTTTVEIRKSSVMTTEITSKVEKSPTTATGNSSCPSTETEEEKAKRLLYCSLCKVAVNSASQLEAHNSGTKHKTMLEARNGSGTIKAFPRAGVKGKGPVNKGNTGLQNKTFHCEICDVHVNSETQLKQHISSRRHKDRAAGKPPKPKYSPYNKLQKTAHPLGVKLVFSKEPSKPLAPRILPNPLAAAAAAAAVAVSSPFSLRTAPAATLFQTSALPPALLRPAPGPIRTAHTPVLFAPY

SEQ ID NO: 41 - DNA, 2397 bp, Homo sapiens
  CDS (111)..(2072)
cccagccccagccccagccccggccccggccccggctcgcgggcgctgcgtctccgcgtacaggaggcggcggcggctcccagtcaccggcccccgccggcgagcgcacgatgcactgcctgggcgccgagtacctggtttctgcagaaggagcccctaggcaaagggagtggcgaccccagatttataggaaatgcacagatacggcatggttattcctgttctttctcttttggactggtttggtgtttatcatgggctactcggtggtggctggagccgcgggaagactcctctttggctatgacagctttggcaacatgtgtggcaagaagaactcccccgtggaaggggcccctctttcagggcaggacatgaccctaaaaaaacacgtgttctttatgaattcctgcaacctggaagtcaaaggtacgcagctcaaccgcatggccctctgtgtatccaactgccctgaagagcagcttgactccctggaagaggtccagttctttgcaaacaccagtgggtccttcctgtgtgtttatagtttgaattccttcaactatacccacagtccaaaagcagactcactgtgtcccaggctaccagttcctccaagcaagtcatttcccttatttaaccgatgtgtccctcaaacacctgagtgctactccctatttgcatctgttttgataaatgatgttgacaccctccaccgaattctaagtggaatcatgtcgggaagagatacaatccttggcctgtgtatcctcgcattagccttgtctttggccatgatgtttaccttcagattcatcaccacccttctggttcacattttcatttcattggttattttgggattgttgtttgtctgcggtgttttatggtggctgtattatgactataccaacgacctcagcatagaattggacacagaaagggaaaatatgaagtgcgtgctggggtttgctatcgtatccacaggcatcacggcagtgctgctcgtcttgatttttgttctcagaaagagaataaaattgacagttgagcttttccaaatcacaaataaagccatcagcagtgctcccttcctgctgttccagccactgtggacatttgccatcctcattttcttctgggtcctctgggtggctgtgctgctgagcctgggaactgcaggagctgcccaggttatggaaggcggccaagtggaatataagcccctttcgggcattcggtacatgtggtcgtaccatttaattggcctcatctggactagtgaattcatccttgcgtgccagcaaatgactatagctggggcagtggttacttgttatttcaacagaagtaaaaatgatcctcctgatcatcccatcctttcgtctctctccattctcttcttctaccatcaaggaaccgttgtgaaagggtcatttttaatctctgtggtgaggattccgagaatcattgtcatgtacatgcaaaacgcactgaaagaacagcagcatggtgcattgtccaggtacctgttccgatgctgctactgctgtttctggtgtcttgacaaatacctgctccatctcaaccagaatgcatatactacaactgctattaatgggacagatttctgtacatcagcaaaagatgcattcaaaatcttgtccaagaactcaagtcactttacatctattaactgctttggagacttcataatttttctaggaaaggtgttagtggtgtgtttcactgtttttggaggactcatggcttttaactacaatcgggcattccaggtgtgggcagtccctctgttattggtagctttttttgcctacttagtagcccatagttttttatctgtgtttgaaactgtgctggatgcacttttcctgtgttttgctgttgatctggaaacaaatgatggatcgtcagaaaagccctactttatggatcaagaatttctgagtttcgtaaaaaggagcaacaaattaaacaatgcaagggcacagcaggacaagcactcattaaggaatgaggagggaacagaactccaggccattgtgagatagatacccatttaggtatctgtacctggaaaacatttccttctaagagccatttacagaatagaagatgagaccactagagaaaagttagtgaatttttttttaaaagacctaataaaccctattcttcctcattgtctttgtcattattgtttgaccaggtaacaatactggaactatattagtttacctttttttgtacaaattaggacagaaaaactcttctaaaaccatgtttatatgcatcaacttacaaagtacactatgtaagaactgaggtaagtttgtaagtgcacaactaataaataaacctttttaagataaggat

SEQ ID NO: 42 - protein, 653 aa, Homo sapiens
MHCLGAEYLVSAEGAPRQREWRPQIYRKCTDTAWLFLFFLFWTGLVFIMGYSVVAGAAGRLLFGYDSFGNMCGKKNSPVEGAPLSGQDMTLKKHVFFMNSCNLEVKGTQLNRMALCVSNCPEEQLDSLEEVQFFANTSGSFLCVYSLNSFNYTHSPKADSLCPRLPVPPSKSFPLFNRCVPQTPECYSLFASVLINDVDTLHRILSGIMSGRDTILGLCILALALSLAMMFTFRFITTLLVHIFISLVILGLLFVCGVLWWLYYDYTNDLSIELDTERENMKCVLGFAIVSTGITAVLLVLIFVLRKRIKLTVELFQITNKAISSAPFLLFQPLWTFAILIFFWVLWVAVLLSLGTAGAAQVMEGGQVEYKPLSGIRYMWSYHLIGLIWTSEFILACQQMTIAGAVVTCYFNRSKNDPPDHPILSSLSILFFYHQGTVVKGSFLISVVRIPRIIVMYMQNALKEQQHGALSRYLFRCCYCCFWCLDKYLLHLNQNAYTTTAINGTDFCTSAKDAFKILSKNSSHFTSINCFGDFIIFLGKVLVVCFTVFGGLMAFNYNRAFQVWAVPLLLVAFFAYLVAHSFLSVFETVLDALFLCFAVDLETNDGSSEKPYFMDQEFLSFVKRSNKLNNARAQQDKHSLRNEEGTELQAIVR

SEQ ID NO: 43 - DNA, 3130 bp, Homo sapiens
  CDS (251)..(1399)
gagtcagtggcttgaaacttttaaaagctctgtgctccaagttacaaaaaagcttttacgaggtatcagcacttttctttcattagggggaaggcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctagatacaggcttgaaagcatcaagattccactcaattgtggagaagaaaaaaggtgctgtagaaagtgcaccaggtgttaattttgatccggtggaggtggtactcaacagccttattcatgaggcttagaaaacacaaagacattagaatacctaggttcactgggggtgtatggggtagatgggtggagagggaggggataagagaggtgcatgttggtatttaaagtagtggattcaaagaacttagattataaataagagttccattaggtgatacatagataagggctttttctccccgcaaacacccctaagaatggttctgtgtatgtgaatgagcgggtggtaattgtggctaaatatttttgttttaccaagaaactgaaataattctggccaggaataaatacttcctgaacatcttaggtcttttcaacaagaaaaagacagaggattgtccttaagtccctgctaaaacattccattgttaaaatttgcactttgaaggtaagctttctaggcctgaccctccaggtgtcaatggacttgtgctactatatttttttattcttggtatcagtttaaaattcagacaaggcccacagaataagattttccatgcatttgcaaatacgtatattctttttccatccacttgcacaatatcattaccatcactttttcatcattcctcagctactactcacattcatttaatggtttctgtaaacatttttaagacagttgggatgtcacttaacattttttttttgagctaaagtcagggaatcaagccatgcttaatatttaacaatcacttatatgtgtgtcgaagagtttgttttgtttgtcatgtattggtacaagcagatacagtataaactcacaaacacagatttgaaaataatgcacatatggtgttcaaatttgaacctttctcatggatttttgtggtgtgggccaatatggtgtttacattatataattcctgctgtggcaagtaaagcacactttttttttctcctaaaatgtttttccctgtgtatcctattatggatactggttttgttaattatgattctttattttctctcctttttttaggatatagcagtaatgctattactgaaatgaatttcctttttctgaaatgtaatcattgatgcttgaatgatagaattttagtactgtaaacaggctttagtcattaatgtgagagacttagaaaaaatgcttagagtggactattaaatgtgcctaaatgaattttgcagtaactggtattcttgggttttcctacttaatacacagtaattcagaacttgtattctattatgagtttagcagtcttttggagtgaccagcaactttgatgtttgcactaagattttatttggaatgcaagagaggttgaaagaggattcagtagtacacatacaactaatttatttgaactatatgttgaagacatctaccagtttctccaaatgccttttttaaaactcatcacagaagattggtgaaaatgctgagtatgacacttttcttcttgcatgcatgtcagctacataaacagttttgtacaatgaaaattactaatttgtttgacattccatgttaaactacggtcatgttcagcttcattgcatgtaatgtagacctagtccatcagatcatgtgttctggagagtgttctttattcaataaagttttaatttagtataaacata

SEQ ID NO: 44 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 45 - DNA, 4101 bp, Homo sapiens
  CDS (350)..(1726)
gtgcttgcggacttcccgccagcgccgagcggccggcttctcgggccaagtggggagcgagcgggagggcgggcaggtggccccgggccgccgcgcccgcgccttggctctgcccccgggagccgagcaagccgctgctccctcgtggtgtgagggcggtgatgtttttcctcccacccacttttgagttccccctcccccctcgcgcgcactctagctctcgccacaacctgccagccccagacctcggacgagagcgccccggggagctcggagcgcgtgcacgcgtggcagacggagaaggccagtgcccagcttgaaggttctgtcaccttttgcagtggtccaaatgagaaaaaagtggaaaatgggaggcatgaaatacatcttttcgttgttgttctttcttttgctagaaggaggcaaaacagagcaagtaaaacattcagagacatattgcatgtttcaagacaagaagtacagagtgggtgagagatggcatccttacctggaaccttatgggttggtttactgcgtgaactgcatctgctcagagaatgggaatgtgctttgcagccgagtcagatgtccaaatgttcattgcctttctcctgtgcatattcctcatctgtgctgccctcgctgcccagaagactccttacccccagtgaacaataaggtgaccagcaagtcttgcgagtacaatgggacaacttaccaacatggagagctgttcgtagctgaagggctctttcagaatcggcaacccaatcaatgcacccagtgcagctgttcggagggaaacgtgtattgtggtctcaagacttgccccaaattaacctgtgccttcccagtctctgttccagattcctgctgccgggtatgcagaggagatggagaactgtcatgggaacattctgatggtgatatcttccggcaacctgccaacagagaagcaagacattcttaccaccgctctcactatgatcctccaccaagccgacaggctggaggtctgtcccgctttcctggggccagaagtcaccggggagctcttatggattcccagcaagcatcaggaaccattgtgcaaattgtcatcaataacaaacacaagcatggacaagtgtgtgtttccaatggaaagacctattctcatggcgagtcctggcacccaaacctccgggcatttggcattgtggagtgtgtgctatgtacttgtaatgtcaccaagcaagagtgtaagaaaatccactgccccaatcgatacccctgcaagtatcctcaaaaaatagacggaaaatgctgcaaggtgtgtccaggtaaaaaagcaaaagaagaacttccaggccaaagctttgacaataaaggctacttctgcggggaagaaacgatgcctgtgtatgagtctgtattcatggaggatggggagacaaccagaaaaatagcactggagactgagagaccacctcaggtagaggtccacgtttggactattcgaaagggcattctccagcacttccatattgagaagatctccaagaggatgtttgaggagcttcctcacttcaagctggtgaccagaacaaccctgagccagtggaagatcttcaccgaaggagaagctcagatcagccagatgtgttcaagtcgtgtatgcagaacagagcttgaagatttagtcaaggttttgtacctggagagatctgaaaagggccactgttaggcaagacagacagtattggatagggtaaagcaagaaaactcaagctgcagctggactgcaggcttattttgcttaagtcaacagtgccctaaaactccaaactcaaatgcagtcaattattcacgccatgcacagcataatttgctcctttgtgtggagtggtgtgtcagcccttgaacatctcctccaaagagactagaagagtcttaaattatatgtgggaggaggagggatagaacatcacaacactgctctagtttcttggagaatcacatttctttacaggttaaagacaaacaagaccccagggtttttatctagaaagttattcaagtgaaagaaagagaagggaattgcttagtaggagttctgcagtatagaacaattacttgtatgaaattatacctttgaattttagaatgtcatgtgttcttttaaaaaaattagctccccatcctccctcctcactccctccctccctccttctctctctctctctctctccctccctctctcacagacacacacacacacacacacacacacacacacacgcacgtccacactcacattaaacgaaagctttatttgaagcaaagctagccaagattctacgttacttttcccttgactggatcccaagtagcttggaagtttttgtgcccaggagagtaaataactgtgaacaagaggctctgcccttaggtctttgtggctgtttaagtcaccaacaatagagtcagggtaaagaataaaaacactttcatagcctcattcattcacttagaagtggtaataatttttccctaatgataccacttttcttttccccctgtacctatgggacttccagaaagaagttaaattgagtaaaatcatcagaaactgaatccatgtaagaaaaaataattgttgaagaaagaagttgatagaattcaaaaaggccatctttttgctttcacatcaataaaatttaccaagtaatagatcagtactcactaatatttttgagaccatagttgtctggtcagaaaaattatattaaattagtaaattctagaagctctttaaaagggaagttttccttcttctccaattataggagttgatttttactttgcaaagtggctcggtcctcatgagcatctgcatgttgactcttcagttaagaaaattgttgttcatttagggaggtggatattctgatgaagatctttatcctaaaccttcctactatccttgtcttattcatcaagcagatattttagtcaagaattccagagaaggctgctcctaaaatgtctacttgcagcccaataccagagcataaactatccattctggggtctggctttagaaatcatctttgtgggaagacctaattcttcacagcaaggatctcaggcatgccttctagatttgttccctctgaggggcaggaatgaactgtagaaatgttttaaggacccagaaaccccatatgtctcattccatgactataggtgagagaattctttcctaagagggtttgataccaataggggaaaatgtaaaatgttcagtctttatgacaacctggcataaaggagtcaattcttatgaaagagacacaagggccttatggccagggtttcttgggacaagactctcaccagcacatcacacacgttctccttggaagagagaagcagtacatcccggttgagaggtcacaaagcattagtttgtgtgtgtgtgtgtgtgtgtgtgtgtgtgtgtgtgtgtgtgtgtgtggtaaaggggggaaggtgttatgcggctgctccctccgtcccagaggtggcagtgattccataatgtggagactagtaactagatcctaaggcaaagaggtgtttctccttctggatgattcatcccaaagccttcccacccaggtgttctctgaaagcttagccttaagagaacacgcagagagtttccctagatatactcctgcctccaggtgctgggacacacctttgcaaaatgctgtgggaagcaggagctggggagctgtgttaagtcaaagtagaaaccctccagtgtttggtgttgtgtagagaataggacatagggtaaagaggccaagctgcctgtagttagtagagaagaatggatgtggttcttcttgtgtatttatttgtatcataaacacttggaacaacaaagaccataagcatcatttagcagttgtagccattttctagttaactcatgtaaacaagtaagagtaacataacagtattaccctttcactgttctcacaggacatgtacctaattatggtacttatttatgtagtcactgtatttctggatttttaaattaataaaaaagttaattttgaaaaatca

SEQ ID NO: 46 - protein, 458 aa, Homo sapiens
MRKKWKMGGMKYIFSLLFFLLLEGGKTEQVKHSETYCMFQDKKYRVGERWHPYLEPYGLVYCVNCICSENGNVLCSRVRCPNVHCLSPVHIPHLCCPRCPEDSLPPVNNKVTSKSCEYNGTTYQHGELFVAEGLFQNRQPNQCTQCSCSEGNVYCGLKTCPKLTCAFPVSVPDSCCRVCRGDGELSWEHSDGDIFRQPANREARHSYHRSHYDPPPSRQAGGLSRFPGARSHRGALMDSQQASGTIVQIVINNKHKHGQVCVSNGKTYSHGESWHPNLRAFGIVECVLCTCNVTKQECKKIHCPNRYPCKYPQKIDGKCCKVCPGKKAKEELPGQSFDNKGYFCGEETMPVYESVFMEDGETTRKIALETERPPQVEVHVWTIRKGILQHFHIEKISKRMFEELPHFKLVTRTTLSQWKIFTEGEAQISQMCSSRVCRTELEDLVKVLYLERSEKGHC

SEQ ID NO: 47 - DNA, 863 bp, Homo sapiens
ctcacctcacagacactcctctctggatcctccagagctataaagacgggccttccaccaccagacaggcgcactctaccaccatgaatccactcctgatccttgcctttgtgggagctgctgttgctgtcccctttgatgatgatgacaagatcgttgggggctacacctgtgaggagaattctgtcccctaccaggtgtccttgaattctggctcccacttctgcggtggctccctcatcagcgaacagtgggtggtgtcagcaggtcactgctacaagccccacatccaggtgagactgggagagcacaatatcgaagtcctggaggggaatgagcagttcatcaatgcagccaagatcatccgccaccccaaatacaacaggattattctgaacaatgacatcatgctgatcaagctctccacacctgccgtcatcaatgcccatgtgtccaccatctctctgcccactgcccctccagctgctggcaccgagtgccttatctccggctggggcaataccctgagctctggggccgactacccagatgagctgcagtgcctggacgctcctgtgctgacccaggctaagtgtaaagcctcctaccctttaaagattaccagcaacatgttctgtgtgggcttccttgagggaggcaaggattcctgccagggtgactctggtggccctgtggtctgcaatggacagcttcaaggaattgtctcctggggctatggctgtgcccagaagagaaggcctggagtctacaccaaggtctacaactatgtggactggattaaggacaccatagctgccaacagctaaagcccctggtcactctgcagtctctataccaataaa

SEQ ID NO: 48 - DNA, 2649 bp, Homo sapiens
  CDS (246)..(827)
ctataaagttctcgctgccccggaggctatgctactgcgaggagccggcgcagggtggcccgggaggggtgagcagggtgccgctggctgctggggtctgcaggtcaccgagtccccaggagaggggactcctaagaagccacctgcctgtgtttacccggcagcgagcgcgcaggcccccgcgaactcctggcagcgctcaggaaaggccgttgcgcctcgcgaaggaaacagagccgttgaccatggttgcaactggcagtttgagcagcaagaacccggccagcatttcagaattgctggactgtggctatcacccagagagcctgctaagtgattttgactactgggattatgttgttcctgaacccaacctcaacgaggtaatatttgaggaatcaacttgccagaatttggttaaaatgctggagaactgtctgtccaaatcaaagcaaactaaacttggttgctcaaaggtccttgtccctgagaaactgacccagagaattgctcaagatgtcctgcggctttcctcaacggagccctgcggcttgcgaggttgtgttatgcacgtgaacttggaaattgaaaatgtatgtaaaaagctggataggattgtgtgtgattctagcgtcgtacctacttttgagcttacacttgtgtttaagcaggagaactgctcatggactagcttcagggactttttctttagtagaggtcgcttctcctctggtttcaggagaactctgatcctcagctcaggatttcgacttgttaagaaaaaactttactcactgattggaacaacagtgattgaagggtcctaaaaagggaaaatatataaagattatttcatgattgggtagtaaaactattcagctagtcagctaaagtcatttgtagtttgccccacctgccctaaataagaaaccccaaatgtagtctcttttctttctgtgtttcacattcatagcaactgcagctaacaggctgattttctggcctttggagaagtgattcaaaatagtgtagattttctgcatagatcccatttttgtacagaattgaatgggatggaataggtaagcaaaagtagaagcccatttgagttttacatttgattccacaatttggtttcaggtaggcttggtaatagactatataaaccagatttgcctattttgattttcatatggcttttttttctgtaagttttcagaggattttttaaatcacagaatcatactaaatgatatttagcctatcaaaacttccaaaagcccacaccaccagttcctgactcaaatttgaagggtttttagacaggagggtaggattaagtaggtgagtttaattaaagcttaaccctaggtaagagtaaatgagaaatattacggcaataatggaactgcttcactgtttcttggtgacttcctcactctaatgttttaaagaggcaacaaaagcttgtggtgccatttcagtaaccacggtgttgttttagatgcctttataagctcagtttcccctgttcttaagtgttgaatactgtctttaaactagaaaaatgcaaaatattgaactgatatttttgtgtgtagttgattactcttccattgagtgaatgatgaatacctgtgaggataggaaattagttctgagatctagtccctctctgattcacttagtaatctatcctcttttcagtattacatgtgcttaatctcagatgaaccatttcaccatggcagtgttatctcatctctgggcttttctgggaattgaagtatctctccttaaccccaattgtcaagggtagtagctgtatactaccactttgaattattgaaacgggtcaatttacgaagtctgcattggctatggagatatggtttatagtacagcctagagaatgaaactcaccgtccagataaccatgcatgcacccagattttttccaccttggatacctgtcactagggaataataaaggcctgattttttgtcttattccaactaagtagatcattatctctttccttttttatgttaatgagagaatttagcctccactcaacaatgttcaattcagcaaggctttcatatccttgctgtgggtcgtggataaggagcttattcaggtttcctgccctagctattagctccacttcacatgctggagaccggcgtagggacagatgtattcatcctggtgttactgaaaaacaggtgtgatcctgttactgatactataagtgacctaaaatgtcactgttcaaattagccagtgttctaacaaactaaactcttcaaatgcttggaaagatactacaaagccaatctttatagaattgggccaagataaatctatgttgttttgcatggctattgttaagctccaaaggttcactgtgtttctgccgctgtcctggagttgtcaccactgactgggcaaggcttcttgggcatggatgtagaactgttgtccttttcccactaacagttatctttgactctcttgcctgttatgcttacaaaatggtgatggcttatggaaggctgttaaattaatattcctgttaaaggaaattaaagtttgtctatttttgacaataaaacattatatatttttaaaaaaaaaaaaaaaaa

SEQ ID NO: 49 - protein, 193 aa, Homo sapiens
MVATGSLSSKNPASISELLDCGYHPESLLSDFDYWDYVVPEPNLNEVIFEESTCQNLVKMLENCLSKSKQTKLGCSKVLVPEKLTQRIAQDVLRLSSTEPCGLRGCVMHVNLEIENVCKKLDRIVCDSSVVPTFELTLVFKQENCSWTSFRDFFFSRGRFSSGFRRTLILSSGFRLVKKKLYSLIGTTVIEGS

SEQ ID NO: 50 - DNA, 7114 bp, Homo sapiens
  CDS (633)..(2300)
gcctccagtgactgtctccaggatttctctcttcctatttcaggaggactctcacaggctcccacagcctgtgttaagctgaggtttcccctagatctcgtatatccccaacacatacctccacgcacacacatccccaagaacctcgagctcacaccaacagacacacgcgcgcatacacactcgctctcgcttgtccatctccctcccgggggagccggcgcgcgctcccacctttgccgcacactccggcgagccgagcccgcagcgctccaggattctgcggctcggaactcggattgcagctctgaacccccatggtggttttttaaacacttcttttccttctcttcctcgttttgattgcaccgtttccatctgggggctagaggagcaaggcagcagccttcccagccagcccttgttggcttgccatcgtccatctggcttataaaagtttgctgagcgcagtccagagggctgcgctgctcgtcccctcggctggcagaagggggtgacgctgggcagcggcgaggagcgcgccgctgcctctggcgggctttcggcttgaggggcaaggtgaagagcgcaccggccgtggggtttaccgagctggatttgtatgttgcaccatgccttcttggatcggggctgtgattcttcccctcttggggctgctgctctccctccccgccggggcggatgtgaaggctcggagctgcggagaggtccgccaggcgtacggtgccaagggattcagcctggcggacatcccctaccaggagatcgcaggggaacacttaagaatctgtcctcaggaatatacatgctgcaccacagaaatggaagacaagttaagccaacaaagcaaactcgaatttgaaaaccttgtggaagagacaagccattttgtgcgcaccacttttgtgtccaggcataagaaatttgacgaatttttccgagagctcctggagaatgcagaaaagtcactaaatgatatgtttgtacggacctatggcatgctgtacatgcagaattcagaagtcttccaggacctcttcacagagctgaaaaggtactacactgggggtaatgtgaatctggaggaaatgctcaatgacttttgggctcggctcctggaacggatgtttcagctgataaaccctcagtatcacttcagtgaagactacctggaatgtgtgagcaaatacactgaccagctcaagccatttggagacgtgccccggaaactgaagattcaggttacccgcgccttcattgctgccaggacctttgtccaggggctgactgtgggcagagaagttgcaaaccgagtttccaaggtcagcccaaccccagggtgtatccgtgccctcatgaagatgctgtactgcccatactgtcgggggcttcccactgtgaggccctgcaacaactactgtctcaacgtcatgaagggctgcttggcaaatcaggctgacctcgacacagagtggaatctgtttatagatgcaatgctcttggtggcagagcgactggaggggccattcaacattgagtcggtcatggacccgatagatgtcaagatttctgaagccattatgaacatgcaagaaaacagcatgcaggtgtctgcaaaggtctttcagggatgtggtcagcccaaacctgctccagccctcagatctgcccgctcagctcctgaaaattttaatacacgtttcaggccctacaatcctgaggaaagaccaacaactgctgcaggcacaagcttggaccggctggtcacagacataaaagagaaattgaagctctctaaaaaggtctggtcagcattaccctacactatctgcaaggacgagagcgtgacagcgggcacgtccaacgaggaggaatgctggaacgggcacagcaaagccagatacttgcctgagatcatgaatgatgggctcaccaaccagatcaacaatcccgaggtggatgtggacatcactcggcctgacactttcatcagacagcagattatggctctccgtgtgatgaccaacaaactaaaaaacgcctacaatggcaatgatgtcaatttccaggacacaagtgatgaatccagtggctcagggagtggcagtgggtgcatggatgacgtgtgtcccacggagtttgagtttgtcaccacagaggcccccgcagtggatcccgaccggagagaggtggactcttctgcagcccagcgtggccactccctgctctcctggtctctcacctgcattgtcctggcactgcagagactgtgcagataatcttgggtttttggtcagatgaaactgcattttagctatctgaatggccaactcacttcttttcttacactcttggacaatggaccatgccacaaaaacttaccgttttctatgagaagagagcagtaatgcaatctgcctccctttttgttttcccaaagagtaccgggtgccagactgaactgcttcctctttccttcagctatctgtggggaccttgtttattctagagagaattcttactcaaatttttcgtaccaggagattttcttaccttcatttgcttttatgctgcagaagtaaaggaatctcacgttgtgagggttttttttttctcatttaaaataaaaaaggaagaaagaaaataattttccttgtaaaatcgggccaaaccccaagacagctacattttcaacaaaaaagcaaacagagaaaaataaatgaactttaacactgtaagttcagcattgacagccaacttccagtgtaagcttttttgtgacaaatccgggtttaaaaatgcttatggggagaaagcttaaagtttgttctaaatgtagtgaaaatgcactgttgtcttggaggtatcatcttgcactctaaccaaacaacaccaaattaatctggaggtactgatactaaatttggaatcccgtctttaatatgagaacacaaattactacaactcagtacctctgtcttactctgcccaatttcaatttagtaatggcaccattattcttaattcccttaatgtctgtccgctgtatccaattggcacagattttccccttcatcttcaattactagtagctacccccatcaattcaccttcctcaagagtctcaattgaaaataatctttgatgatacaatctctgcttttgccacaggtttgacattaactagacgtttctgcccctgtgctcattaccctcccaaaacaatagtttttttaatgaattgaaacacaattagatccattcctgagttttcatttttaactatttactttgtgtcagttggtttctgcatcacaagggcattctcttacaaaacaactcacaacagaaaaaaaaagtcaagacaaaaaatatatatatatagtattgacatggagatttctttcacttttttagtcttagtatgatcatctatttttatatctatgtatatataaatcacagattttaacttcttaattccaagctcagggttgacacacctttgtaagaaaatgttttgtcaaccagctcttcttgttttgtgctgctcaaagaagggattcctcagtgatcggtgagcaccaagaatcaagaaagagaactttttatgtatctaactgtccatttattaaaaatttgccagggcacacccttctgcatgagtgtgcttcagcctgggtgctccagactacaccaaactcatgggatgagccattggtgtgcagggctttaccggtgatggaaactgtatgtgtaaagaatgttattcatgaaaaaccattgctttcatgtaaaacaccttcggcctaggattcctgcctggcacttggatacattacctaactcaataggccagtttagagccacagcctaacctgttcaccaagtaggccaaagccaggcctttcgctccccctgacagagagaagcaaaataaacccaactgggggctttatgaatacagttggctatccatatgtttcttagtttttatgatgcatctggagtagggaaaagtaatgtttcaagacaccatagtaggccagttacattttaattaactcatcgacagtattgacacgtaaatggtatttttcaatctgttctcatcaaaattaaaattagttattgatttagtcatcacatttaagtagaacacatgtggcacaatagtattaagtatatttgagcacttttgcaagacagaaagtatttacaagcatctattgctactttatgcctaatgaaatataaaatgtaggatgatgagagactttgtggcattctttcatttaaacctttatgaaattaaccatctagttcatcattacatactcctagaaaaccatccatttcactgcccacattttggcttcctaccacaaattccacataagacttctctatgaaggaccagtcatttacattgctcatttcattctgtgttcagaagtcagtattgtctttctccaataattaaggtgcaatacaaattaaatcaaccttgattttccagactatttgaataataacttgcttttatggctttaaactccaggtctttcctttttaaaatatttctgcattctcttttatgaactagatcaggatgccttgtttttcctgtgctttcacccccaaaaggttttgtgcatgtgtatgaatacacataatattagtgaatgtgaacccatgttctatacgcactaaacacacagttaatatataaacgcctgcttcatttgtttcaacaccttcattatcaatatgattcatattgatgagatatagtgatgcataatgagccaggcagagagctgaaatgatatcagttaatgatgtcacagtgttagcaccctgcatggatgtggtcaaaatatcattcaagttgtacttcctagtttttatcatcatactactatgtcatatgattttcagactaatttaacatgagaaatgttttggccctaaaggatttcacttaacctagttaccctatttcagtaagtattttggaattcaaccctcgaattattttttctcatttcagcatagtgataggggatgcaatgaggcttcattattttttatgacctgcccctcatttgctctgatgttccctaaattctgtaatcatatcataacttttgttatgaatagagaggaatgggctcactgaaacctgacactagaaattggtgggtgatgctcataactgcaaacacttagcttattgaagtgcctctatttacatgttctttagttataatatgtatttttctaacagaaatacacgtctgtaattggtatatattatactttgtatgtgtcacaacaaaagctaaacagaggctaaagtctttagcagagaagaatgaattacaaatttaccaataagagctctgtttatggttctgtattgaaggacaaccatgacaaccttctgtcattttcacgaaggccaagttggtactttgattatagccatcgtagaacattagcactagaaatctgtctcccttacaatgacttacctagctagcatcaagtaacttgtatcacctgctttaaatgtaagggatagaaaatactattttgtctacactgaaaaacattattattcattgataagtagtaataattataaaagtaacattctttaaagcaaacacattgaaaatattattacaggcagaaccctgatttttaatttatattccctgctgcagacattagccttctttaattcttttgccattacatacatgtttcggctacagactgaacacgaactaaagtccctaaggaggaaaaaaccttaacatattcctgagatgtgattcttttattacttctttttgcaaagctaattatcaaatgatttgattaaacctagacgcattaggaaatgcaagttttacctaaaacttgcagaaatttaaacacatttccatttacttactaacgaacagacctgatttttatttaaaaacacaatttagctttgccaacagaagagtaaaggaaaccataacttataaatttgtaatgtgttcctccttgttggagacattagccagtgtttagttttagaccaacttatacaaagagagtattgatttgtacacattctggagacttgctatatcatggtttgctatggcacagactcagcttagcatggggacattttgaacttcattcaaagttgagtagttactggaacctttgacattgccttgtaatgaggtacttccaaaaaaagacccctaacaatggcataatagtgaggtctctctgtgcatatacataatatatatgcaggataaatgtgtgcaccaatcaaatctaaaattttatgtgactgtcaaatgaatattatttgggttaagatttttcatttctgatttggatagaaaattgctattaatcttgtattaaaatagtttttaaaaatctaccagtgccctttctgcattttcttaattattgtcatagtctaaatttcagagccatggaattttttgtgtttgagacatttttagaagcttcaatattaaaaaacaacaacaaacctcgtatattaaaacagtgccactctcctaagtacactcagcatctgaaatcaggaactatttgaactttagaacatttagagcttcccaaatgtgtatggaaaatactgtagcgctgttcttttgtactgatttcttctcagaggataaatttagtgacttgaaaactgatgtcaatactttataaatgaggtcagaaaataagatcaaatgtgaaaagactaataatttcccaatttagccaattgcattgatttttatacttctgaaatgtttccatcctctcaggcatgctaaatattgtataacctgctaaagatttatttcacaaatgcttattgaactcttattctctataaaccctgctctaggggatttatcactggctgtgaaaaaccaccagttgaaaattattcattccttaatgcagctaatcataattattacagataaataatgtatttcaagaatgatcttataaataaaggaacccatacaaacaaaaaaaaaaaaaa

SEQ ID NO: 51 - protein, 555 aa, Homo sapiens
MPSWIGAVILPLLGLLLSLPAGADVKARSCGEVRQAYGAKGFSLADIPYQEIAGEHLRICPQEYTCCTTEMEDKLSQQSKLEFENLVEETSHFVRTTFVSRHKKFDEFFRELLENAEKSLNDMFVRTYGMLYMQNSEVFQDLFTELKRYYTGGNVNLEEMLNDFWARLLERMFQLINPQYHFSEDYLECVSKYTDQLKPFGDVPRKLKIQVTRAFIAARTFVQGLTVGREVANRVSKVSPTPGCIRALMKMLYCPYCRGLPTVRPCNNYCLNVMKGCLANQADLDTEWNLFIDAMLLVAERLEGPFNIESVMDPIDVKISEAIMNMQENSMQVSAKVFQGCGQPKPAPALRSARSAPENFNTRFRPYNPEERPTTAAGTSLDRLVTDIKEKLKLSKKVWSALPYTICKDESVTAGTSNEEECWNGHSKARYLPEIMNDGLTNQINNPEVDVDITRPDTFIRQQIMALRVMTNKLKNAYNGNDVNFQDTSDESSGSGSGSGCMDDVCPTEFEFVTTEAPAVDPDRREVDSSAAQRGHSLLSWSLTCIVLALQRLCR

SEQ ID NO: 52 - DNA, 2054 bp, Homo sapiens
actgggctctctaaattcatttcagctctaggtaaggttaaactcttctcccgtcttctggattttcaggttctccagggccgatgtatgtttggagatggatttccccctctcacacttcaggcactcagttttttggcagtctcatggagttttcagtggcaagccgcttatttcaaatgggatttggaaaggattccgtgatggttaatattgagtgtcaacttgattggattgaaggatgcaaagtattgttcctgggtgtgtctgtgagggtgtcaccaaaggagatgaacatttgaatcagtgagctgggagacacaaacccatactcaatcttgggggacaccatctaatcagctgccagtgcaactaggataaaatcaggcagagaaatttggaaagactagactggctgagtcttctggccttcatctttctcccatgctagatgcttcctgacctcgaatatcagactccaagttctttagcttttggactcttggacctttggccacagacggaaggctgcacttttgaagttttgggacttggactggcttccttgctcctcagcttacagatggcctattgtgggacttcaccttgtgattgtgatttcaaaccttctgggtttcttcccagcgctaaatcctgtcgactggattcactaggatgaatagagaaagaagtgttacattttaagataatttcttttctaaacacacagaaagttttgaaaccaatggagagatcaacacaagctttggagaggagatcaaaatatctgcttcactggaaggagttttggattgctcaacaaggttcagatctgtggcataatcaggcttcctaatcctgaaccaaagttgttgttgtatctcgtagttgtcacctgagagctgactgacctgtttcaagcatatgtgagatgccttctggaaatgcccggaagcatatagctaatgggatgaggcatttcaaggttgaacttcagcaagagcaagtgggataaagggcaaaaagcagcacattcctttcaaaatgtccccattttaaactgcaggctagagtagacaatggaaatatatgacatataaagaaactggtgagattattaagcagagggccttctggggagcattaaagagtgagcaagaaaaatttctcagacatggtgattctagaattgaaaaatcatagactctttctcttctctctcctttgagagtttccagatgaattcttggatctggctactgagcaaatatatgaataataacctaggatcagctgggtataagaggtttccaccagaagtccatgaaaaccaatgtttttaagcatctattgggcaactaccataaacaatcagcaagatgtttaaacagttgattctgccggcggcccaaccagtttttaactgtatcacgaaaactggcctgggggtacagtctgagattattccagccctgcttttacaccgccagaaattccctttctgttacaaatttctaccccagagaggtttgcatattcttggcattttcactgtgcatcagtgaatcagctagcctacgggccaaggagaggggaaaaaaatcccaaataaaactcaacccagttaggatgtcttcatctatttttcagtgggaaaaaatacagctggactcagtgacttcagtcaaaagcaaacattactagttgcttcatttttctaatttttatttttgactctcaagttttggagggcataataaagacatagcctcagcaaagtcacttagaagattgagttcgttattttgcaaagcttgggtagtgacacttctgcaaaatgcacaagcacatgagcagagagatgtgtgaattcaggaaagcagccaaatgcacatcagagctgaggaatgtcacagcgaaatgaagaaaacttgtaaacacctatctgtatttttaggtagagaatgttctaacatattgccaatgggtgctgcctgtgtgttccttcccctaagctttaaactcaactgatattaaaggtgatttgaaaacaa

SEQ ID NO: 53 - DNA, 1815 bp, Homo sapiens
  CDS (39)..(1379)
tcattactacagggcaggaagggcacgttctcatcagcatgagggagtgcctttccatccacatcggtcaagctggcatccagattggggacgcctgctgggaactctattgcctggaacatggaatccagccaaatggcgttgttcttgacactcaacaggatcagctggaaaatgcaaaaatggagcacacaaatgcatctttcgataccttcttctgtgagacaagagctgggaagcatgtgcctagagcactcttcgtggacttggagccaactgttatagatgggatccggacgggccagcaccgttcactcttccaccccgagcagctccttagcggaaaggaggatgctgctaacaattacgcgcgaggccgttactctgtggggtcggaggtcatcgaccttgtgctggagaggacccggaagctggcagaacagtgtggtggacttcagggatttttgattttccgaagctttggaggaggcactggttcagggtttacgtctctcttaatggagaggctcacaggagaatatagcagaaagactaagctggagttctcggtctacccagcccccaggatctccactgctgtggtagagccttataactctgtcctcaccacccactccaccacagagcacacggactgtaccttcatggtggacaacgaggccgtctatgatatatgccatcgtaaactcggtgttgaatgcccctctcatgccagcatcaatagattggtggttcaggtggtatcttccatcactgcctccctccggtttgaagggcccttgaatgtagacctaattgaattccagaccaacctggtaccttatccgagaatacatttccccatgacagccttcgcccccatcgtctctgctgacaaagcctaccatgagcagttctctgtgtcagacatcaccactgcctgctttgagtcctccaaccagctggtcaagtgtgatcctcggcttgggaagtacatggcctgctgcctactctatagaggggatgtggtccccaaggaagtgaatgcagcaatcgcagccacgaagtcgaggcactctgttcagtttgtagattggtgtccaactggtttcaaggtgggcatcaacaatcggccgcccacggtgatgccgggtggggacctggccaaagtccaccggtccatctgcatgctgagcaacaccacggcgattgtggaggcctgggcccgcctggaccacaagtttgacctcatgtacgccaagagagcatttctgcactggtacctcagagaaggcatggaagaagcagagttcttggaggccagggaagatctggcagccctggagagggactatgaggaagtggcgcaaagtttctgaggcatgtatgatgggtgcaaatgaatgtcaagttaaaatggcatgttttcttttcaagccgttatatgcctagtgggtagttcccctgatgagcagaaaaatgaactcaaatcaggcaagactctaggttccacactaaattaagtgggtcagtaccaacaatgaacacattatttcctggtctactggtacaagtcagctctgctacctgggagcctttggaagcttagagtcctttgaactgctgagtcacttctaggtgggatttttttgttttaccaagagacaatgaaatcctgaagtttatgcttttcctttctggtaccggaagtagaaaacagcacatgttgattacgaatgttttttcccccatgtctacattttttacactgattaaatcataaatattttcagtatgaaaaaaaaaaaaaaaaaa

SEQ ID NO: 54 - protein, 446 aa, Homo sapiens
MRECLSIHIGQAGIQIGDACWELYCLEHGIQPNGVVLDTQQDQLENAKMEHTNASFDTFFCETRAGKHVPRALFVDLEPTVIDGIRTGQHRSLFHPEQLLSGKEDAANNYARGRYSVGSEVIDLVLERTRKLAEQCGGLQGFLIFRSFGGGTGSGFTSLLMERLTGEYSRKTKLEFSVYPAPRISTAVVEPYNSVLTTHSTTEHTDCTFMVDNEAVYDICHRKLGVECPSHASINRLVVQVVSSITASLRFEGPLNVDLIEFQTNLVPYPRIHFPMTAFAPIVSADKAYHEQFSVSDITTACFESSNQLVKCDPRLGKYMACCLLYRGDVVPKEVNAAIAATKSRHSVQFVDWCPTGFKVGINNRPPTVMPGGDLAKVHRSICMLSNTTAIVEAWARLDHKFDLMYAKRAFLHWYLREGMEEAEFLEAREDLAALERDYEEVAQSF

SEQ ID NO: 55 - DNA, 3890 bp, Homo sapiens
  CDS (49)..(2403)
gcacctgggcacctgggcagccgccgcggcgctggctagacgtgcgcgatggagggcgacggcgggaccccatgggccctggcgctgctgcgcaccttcgacgcgggcgagttcacgggctgggagaaggtgggctcgggcggcttcgggcaggtgtacaaggtgcgccatgtccactggaagacctggctggccatcaagtgctcgcccagcctgcacgtcgacgacagggagcgcatggagcttttggaagaagccaagaagatggagatggccaagtttcgctacatcctgcctgtgtatggcatctgccgcgaacctgtcggcctggtcatggagtacatggagacgggctccctggaaaagctgctggcttcggagccattgccatgggatctccggttccgaatcatccacgagacggcggtgggcatgaacttcctgcactgcatggccccgccactcctgcacctggacctcaagcccgcgaacatcctgctggatgcccactaccacgtcaagatttctgattttggtctggccaagtgcaacgggctgtcccactcgcatgacctcagcatggatggcctgtttggcacaatcgcctacctccctccagagcgcatcagggagaagagccggctcttcgacaccaagcacgatgtatacagctttgcgatcgtcatctggggcgtgctcacacagaagaagccgtttgcagatgagaagaacatcctgcacatcatggtgaaggtggtgaagggccaccgccccgagctgccgcccgtgtgcagagcccggccgcgcgcctgcagccacctgatacgcctcatgcagcggtgctggcagggggatccgcgagttaggcccaccttccaagaaattacttctgaaaccgaggacctgtgtgaaaagcctgatgacgaagtgaaagaaactgctcatgatctggacgtgaaaagccccccggagcccaggagcgaggtggtgcctgcgaggctcaagcgggcctctgcccccaccttcgataacgactacagcctctccgagctgctctcacagctggactctggagtttcccaggctgtcgagggccccgaggagctcagccgcagctcctctgagtccaagctgccatcgtccggcagtgggaagaggctctcgggggtgtcctcggtggactccgccttctcttccagaggatcactgtcgctgtcctttgagcgggaaccttcaaccagcgatctgggcaccacagacgtccagaagaagaagcttgtggatgccatcgtgtccggggacaccagcaaactgatgaagatcctgcagccgcaggacgtggacctggcactggacagcggtgccagcctgctgcacctggcggtggaggccgggcaagaggagtgcgccaagtggctgctgctcaacaatgccaaccccaacctgagcaaccgtaggggctccaccccgttgcacatggccgtggagaggagggtgcggggtgtcgtggagctcctgctggcgcggaagatcagtgtcaacgccaaggatgaggaccagtggacagccctccactttgcagcccagaacggggacgagtctagcacacggctgctgttggagaagaacgcctcggtcaacgaggtggactttgagggccggacgcccatgcacgtggcctgccagcacgggcaggagaatatcgtgcgcatcctgctgcgccgaggcgtggacgtgagcctgcagggcaaggatgcctggctgccactgcactacgctgcctggcagggccacctgcccatcgtcaagctgctggccaagcagccgggggtgagtgtgaacgcccagacgctggatgggaggacgccattgcacctggccgcacagcgcgggcactaccgcgtggcccgcatcctcatcgacctgtgctccgacgtcaacgtctgcagcctgctggcacagacacccctgcacgtggccgcggagacggggcacacgagcactgccaggctgctcctgcatcggggcgctggcaaggaggccatgacctcagacggctacaccgctctgcacctggctgcccgcaacggacacctggccactgtcaagctgcttgtcgaggagaaggccgatgtgctggcccggggacccctgaaccagacggcgctgcacctggctgccgcccacgggcactcggaggtggtggaggagttggtcagcgccgatgtcattgacctgttcgacgagcaggggctcagcgcgctgcacctggccgcccagggccggcacgcacagacggtggagactctgctcaggcatggggcccacatcaacctgcagagcctcaagttccagggcggccatggccccgccgccacgctcctgcggcgaagcaagacctagctggctgcctgcggagaccgggggtccacgtggggctcttgtcctgtcctgtgttcctcgtggggatggaacgatcctgcgtggggccctgttgtggcttacctaaatgttaaccaagcagaggtgacatggtgccatcaggaggcggctgctgctgaccggagtgtcccctccaggtgaagctggctcaggtgcacatgcccgctccatcatcgatctaggcacctgctgtctgaagggaccgtgggtcagaatcatttcgttgtgctcctaatgggtcgctgaggctggtctctcagtgatgaagccccaggcgtggaagcatccactctctcctgaggcgagccaccttgggttgctggagctcaccagtcttgagggaggtgcaggggaaactgtgttttttatcttcatacatgacggtgggcagagaggcctgtcttaaagtttccatggaattgttttataaaatatcttaagagatgaataccttatcagctgttgcttgaaacctgttaaaaatgttcataacattggatagtctagtctctaaatgatgactaagtagtggggttggctttgaaaacaatgttttatgcaacaaggaacgaatggtagcagccagctttgcggggcgtatgtgtggccagctcttaaccattccagtctattacttgggtgagtccttgtggacaaccacacacacgtgcccacatggtactagctgccgttcgtttctcgttgcctaagatgttttggcaactctagagccacaggcctaagtcattaaaaaattctccctttgtaacctcagtgctggggactgaggcgagccccctcaggtcgctggagtgcaccagtcttggggaagaggtgcaggagaagctgtgttttttatctccacacgcagtatgaagataaaattacatagtattacctagacatagacagtattacctaggtagatgcactgctcacctgcgcccttcccagctctcatttttgttaggtgatttgggatagggatagtgttttggggtatggggggagtgtttctgacctgctttgcagacgtgcctccgcacctcagcagtttggggtgtggccccagggcggttcttggatgtaaaagatgtggccatctagcctcgtaacttcactgtcacctgtgtcccatagggtgccttctgaatactgttattagaataagtttgttgcagaacgtgaccctgcgtgcaaacatgtaccgtggcctggtatatgatagagattgatattaatgtaccatgtatgttaatgtgaatctgtgggcaggatacttttccatggcaggaaatatccaagctgttgaaactggctatgttttaatatgcctcattgtgcctttactgttgtgtggactgcgtgagggacaagaagttccatttgatgtcaataaagcaaagtacttgcctacttttttgaagctgaaaaaaaaaaaaaaaaaa

SEQ ID NO: 56 - protein, 784 aa, Homo sapiens
MEGDGGTPWALALLRTFDAGEFTGWEKVGSGGFGQVYKVRHVHWKTWLAIKCSPSLHVDDRERMELLEEAKKMEMAKFRYILPVYGICREPVGLVMEYMETGSLEKLLASEPLPWDLRFRIIHETAVGMNFLHCMAPPLLHLDLKPANILLDAHYHVKISDFGLAKCNGLSHSHDLSMDGLFGTIAYLPPERIREKSRLFDTKHDVYSFAIVIWGVLTQKKPFADEKNILHIMVKVVKGHRPELPPVCRARPRACSHLIRLMQRCWQGDPRVRPTFQEITSETEDLCEKPDDEVKETAHDLDVKSPPEPRSEVVPARLKRASAPTFDNDYSLSELLSQLDSGVSQAVEGPEELSRSSSESKLPSSGSGKRLSGVSSVDSAFSSRGSLSLSFEREPSTSDLGTTDVQKKKLVDAIVSGDTSKLMKILQPQDVDLALDSGASLLHLAVEAGQEECAKWLLLNNANPNLSNRRGSTPLHMAVERRVRGVVELLLARKISVNAKDEDQWTALHFAAQNGDESSTRLLLEKNASVNEVDFEGRTPMHVACQHGQENIVRILLRRGVDVSLQGKDAWLPLHYAAWQGHLPIVKLLAKQPGVSVNAQTLDGRTPLHLAAQRGHYRVARILIDLCSDVNVCSLLAQTPLHVAAETGHTSTARLLLHRGAGKEAMTSDGYTALHLAARNGHLATVKLLVEEKADVLARGPLNQTALHLAAAHGHSEVVEELVSADVIDLFDEQGLSALHLAAQGRHAQTVETLLRHGAHINLQSLKFQGGHGPAATLLRRSKT